SEQUENCE LISTING (342 sequences)

SEQ ID NO: 1 - DNA, 364 bp, Homo sapiens
caggtgcagctgcaggagtccggctcaggactggtaaagccttcacagaccctgtccctcacctgcgctgtctctggtggctccatcaacagtggtggttactcctggagttggatccggcagccaccagggaagggcctggagtggattggctacttctatcgtggtgggaggaccttctacaagccgtccctcaagagtcgactcaccatatcagtagacaggtccaacaaccagatctccctgaacctgaactctgtgaccgccgcggacacggccgtgtatttctgtggcgtatactacggtgacgacactgcatccttccagcactggggccagggcaccctggtcaccgtctcctcag

SEQ ID NO: 2 - DNA, 366 bp, Homo sapiens
gaagtgcagctggtggagtctgggggaggcgtggtccagcctggggggtccctgagactcgcctgtgcggcgtctggattcaccttcagtaccaatgccatgcactgggtccgccaggctccaggcaaggggctggagtggctggcatttatctggtatgttggaagtactgaacactatgcagactccgtgaagggccgattcaccatctccagagacaattccaagaacacggtgtatctgcaaatgaagagcctgagagctgaggacacggctgtgtattattgtgtcgccccctcttgtactagtagcaactgctatgcctttgacgactggggccagggaaccctggtcaccgtctcctca

SEQ ID NO: 3 - DNA, 379 bp, Homo sapiens
gaggtgcagctggtggagtctgggggaggcctggtcaagcctggggggtccctgagactctcctgtgcagcctctggattcaccttcagtagttatagcatgaactgggtccgccaggctccagggaaggggctggagtgggtctcatccattagtagtagtagtagtcacatatactacgcagactcagtgaagggccgattcaccatctccagagacaacgccaagaactcactatatctgcaaatgaacagcctgagagccgaggacacggctgtgtattattgtgcgagagccccagaaagatttttggagtggttatcgccatactggtacttcgatctctggggccgtggcaccctggtcaccgtctcctcag

SEQ ID NO: 4 - DNA, 389 bp, Homo sapiens
aggtgcagctggtgcagtctggacctgaggtaaagaagcctggggcctcagtgaaggtctcctgcaaggcctctgattacacctttagtagttatggtatcagctgggtgcgacaggcccctggacaagggcttgagtggatgggatggatcagcgcttacaatggtaacccaaagtatgcacagaagctccagggcagagtcaccttgaccacagacacattcacgagcacagcctacatggaactgaggagcctgaaatctgacgacacggccgtgtattactgtgcgagagagaatttgtggtttagaagtggctggtacacgggtggggacaactactacgctatggacgtctggggccaagggaccacggtcaccgtctcctca

SEQ ID NO: 5 - DNA, 348 bp, Homo sapiens
caggtgcagctgcaggagtcgggcccaggactggtgaagccttcggggaccctgtccctcacctgcgctgtctctggtgactccatcagcaatgattactggtggagttgggtccgccagcccccagggaagggactggagtggattggggagatctatcatagtgggaccaccaactatgacccgtccctcaggactcgagtcaccttatctatagacaagtccgagagccatttctccctgaaggtgagctctgtgaccgccgcggacacggccgtttattactgtgtgggccgctctacgatgacccttttctggggccagggaaccctggtcaccgtctcctca

SEQ ID NO: 6 - DNA, 366 bp, Homo sapiens
gaagtgcagctggtggagtctgggggaggcgtggtccagcctgggaggtccctgagactctcctgtgcagcgtctggattcaccttcagtgactatggcatgcactgggtccgccaggctccaggcaaggggctggagtgggtggcaattatatggtatgatggaagtagttactactatgcagactccgtgaagggccgattcaccatctccagagacaattccaagaacacgctgtttctgcaaatgaacagcctgagagccgaggacacggctgtttattactgtgcgagagatcgacttggtagaagtggctggtgtgactttgactactggggccagggaaccctggtcaccgtctcctca

SEQ ID NO: 7 - DNA, 366 bp, Homo sapiens
gaggtgcagctggtggagtctgggggagacttggtcaagcctggagggtccctgagactctcctgtgcagcctctgaattcaccttcagtgacttctacatgggctggatccgtcaggctccagggaaggggctggagtggatttcatacattagtcccggtggttcgatcatatactacgcagactctgtgaacggccgattcaccacttccagggacaacgccaagaactcactgtatcttctaatgaccagtctgagaaccgaggacatggccatgtattactgtgcgagagatctggatagcggcaactccttccgctactttgactactggggccagggaaccctggtcaccgtctcctca

SEQ ID NO: 8 - DNA, 371 bp, Homo sapiens
aggtgcagctggtgcagtctggggctgaggtgaagagtcctggggcctcagtgaagctctcctgtaaggcttctggaagcatttacgtcgcctcttatatacactgggtgcgacaggtccctggccaagggcttcagtggctgggacgggtcaaccctaaaagtggtggcacaagctatgggccgaattctcagggcagggtcaccatgaccagggacacgtctagcgtcacagtttacgtggaactgtccaggctgacatttgacgatacggccgtatattactgtgcgattctgggcgactggaatggctttgtctaccaccagggaacggacgtctggggccaagggaccacggtcaccgtctcctca

SEQ ID NO: 9 - DNA, 363 bp, Homo sapiens
caggtgcagctgcaggagtcgggcccaggactggtgaagccttcggagaccctgtccctcacctgcactgtctctggttactccatcagcagtactaattactactggggctggatccgccagcccccagggaaggggctggagtggattgggagtatacaatctagtgggagcacctactacaacccgtccctcaagagtcgactcaccatatccgcagacacgtccaagaaccagttctccctgatgctgagctctgtgaccgccgcagacacggctatgtattactgtgcgagatcgaactggaacggtgggggggcgttcgacccctggggccagggaaccctggtcaccgtctcctca

SEQ ID NO: 10 - DNA, 342 bp, Homo sapiens
gacatcgtgatgacccagtctccagactccctggctgtgtctctgggcgagagggccaccatcaactgcaagtccagccagaatattttatacagctccaacaataagaactacttagcttggtaccagcagaaaccagggcagcctcctaagctgctcatttactgggcatctactcgggaatccggggtccctgaccgattcagtggcagcgggtctgggacagatttcactctcaccatcagcagcctgcaggctgaagatgtggcagtttattactgtcaacaatattatagtactcctccgtggacgttcggccaagggaccaaggtggaaatcaaa

SEQ ID NO: 11 - DNA, 369 bp, Homo sapiens
gaggtgcagctggtggagtctgggggaggcctggtcaagccgggggggtccctgcgactctcctgtgcagcctctggattcaccttcagtagctatagcatgatctgggtccgccaggctccagggaaggggctggagtggctctcatccattactagtagtagtagtcacatattctacgcagactcagtgaagggccgattcaccatctccagagacaacgccaagaactcactgtatctgcaagtgaacagcctgagagccggggacacggctatgtactactgtgcgagagtaaacggcgaactgggaccgacagaatactaccttgactactggggccagggaaccctggtcaccgtctcctca

SEQ ID NO: 12 - DNA, 324 bp, Homo sapiens
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatgtatggtgcgtccaccagggccactggtatcccagccaggttcagtggcagtgggtctgggacagagttcactctcaccatcagcagtctgcagtctgaagattttgcagtttattactgtcagcagcataatgactggcctccgcggacgttcggccaagggaccaaggtggaaatcaaa

SEQ ID NO: 13 - DNA, 351 bp, Homo sapiens
gaggtgcagctggtggagtctgggggaggcttggtccagcctggggggtccctgagactctcctgtgcagcctctggattcacctttagtagctactggatgagctgggtccgccagactccagggaaggggctggagtgggtggcccacataaagcaagatggaagtgagaaacactatgtggactctgtgaaggaccgattcaccatctccagagacaacgccaagaactcactgtatctgcaaattaacagcctgagagccgaggacacggctgtgtattactgtgcgagaggagggagttgggatgcctactactggggccagggaaccctggtcaccgtctcctca

SEQ ID NO: 14 - DNA, 333 bp, Homo sapiens
gatattgtgatgacccagactccactctctctgtccgtcacccctggacagccggcctccatctcctgcaagtctagtcagagcctcctgcatagtagtggaaagacctatttgtattggtacctgcagaggccaggccagtctccacagctcctaatctatgaagtttccagccggttctctggagtgccagataggttcagtggcagcgggtcagggacagatttcacactgaaaatcagccgggtggaggctgaggatgttggggtttattactgcatgcaaggtatacaccttcccactttcggcggagggaccaaggtggagatcaaa

SEQ ID NO: 15 - DNA, 369 bp, Homo sapiens
caggtgcagctgcaggagtcgggcccaggactggtgaagccttcacagaccctgtccctcacctgcagtgtctctgttgactccatcaccagtggtgaataccagtggacctggatccgccagcgcccagggaagggcctggagtggattgggcacatccattacagtgggagcacccactacaacccgtccctcaagagtcgatttatcatctcggtagacacgtccaagagccagttctccctgaagctgagctctgtgactgccgcagacacggccgtgtactactgtgccagacaatggtttagtagcaattggtacaactggttcgacccctggggccagggaaccctggtcaccgtctcctca

SEQ ID NO: 16 - DNA, 331 bp, Homo sapiens
cagtctgtgctgactcagccacccccagcgtctgggacccccggtcagagggtcaccatctcttgttctggaagcagctccaacatcggaagtaatactgtaaactggtaccagcatctcccaggaacggcccccaaactcctcatccatactaataatcagcggccctcaggggtccctgaccgattctctggctccaagtctggcacctcagcctccctggccatcagtgggctccagtctgaggatgaggctgattattactgtgcagcatgggatgacagcctgactggtgtggtattcggcggagggaccaagctgaccgtcctag

SEQ ID NO: 17 - DNA, 363 bp, Homo sapiens
caggtgcagctgcaggagtcgggcccaggactggtgaagccttcggagaccctgtccctcacctgcactgtctctggtggctccatcaggagttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgaactctgtgaccgccgcggacacggccgtgtattactgtgcgaggtccgcctctttttcagtcctgaaccccaaatttgactactggggccagggaaccctggtcaccgtctcctca

SEQ ID NO: 18 - DNA, 387 bp, Homo sapiens
caggtacagctgcagcagtcaggtccaggactggtgaagccctcgcagaccctctcatttacctgtgccatctccggggacagtgtctctagcaacagtgttgcttggaactggatcaggcagtccccatcgagaggccttgagtggctgggaaggacatactacaggtccaagtggtataatgattatgcagcatctgtggaaagtcgaataaccatcaacccagacacatccaagaaccagttctccctgcagttgaactctgtgactcccgaggacacggctgtgtattactgtgcaagagacccttggaggggtggaactacccttcggacaatatttgcttttgatatctggggccaagggacaatggtcaccgtctcttca

SEQ ID NO: 19 - DNA, 368 bp, Homo sapiens
aggtgcagctggtgcagtctggggctgaggtgaagaagcctggggcctcagtgaaggtctcctgcaaggcttctggatactccttcaccggctactgtatgcactgggtgcgacaggcccctggacaagggcttgagtggatgggacggatcaaccctctcagtggtgacacacactatgcacacaagtttcagggcagggtcaccttgaccagggacacgtccatcagcacagcctacatggagctgagcaggctgagatctgacgacacggccgtgtattattgtacgagagatctgggggtgagtgattaccctcaggaccactttgactactggggccagggaaccctggtcaccgtctcctca

SEQ ID NO: 20 - DNA, 363 bp, Homo sapiens
gaagtgcagctggtggagtcggggggaggcgtgctccagcctgggaggtccctgagactctcctgtgaagcctctggattcaggttcagtaactatggcatgcactggatccgccaggctccaggcaaggggctggattgggtggcagttatatcatatgagggaaggaaacaatactacgcaaactccgtgaagggccgattcatcatctccagagacaattccaagaatacactgtatttggaaatgaggtttctgagagctgaggacacggctatttattattgtgcgaaagaggagtcggagtatagaagtacggccgatgactactggggccagggaaccctggtcaccgtctcctca

SEQ ID NO: 21 - DNA, 369 bp, Homo sapiens
caggtgcagctgcaggagtcgggccccggactggtgaggccttcggagaccctgtccctcacctgcactgtctttggtggctccatgactagtagcagttacgactggggctggatccgccagcccccagggaaggggctggagtggattgggtatatctcttatactgggagcatctaccagaacccctccctgaagagtcgagtcaccatgtccgtagacacgtccaagaatcagttctccctgacggttacctctgtgaccgccgcagacacggctgtatacttctgtgcgagggggatcagggacttaggatacgccagatatgccgactactggggccagggaaccctggtcaccgtctcctca

SEQ ID NO: 22 - DNA, 357 bp, Homo sapiens
caggtgcagctgcaggagtcgggcccaggactggtgaagccttcacagaccctgtccctcacctgcgctgtctctggtggctccatcagcagtggtggttcctactggaactggatccgccagcccccagggaagggcccggattggattgggtgcgtccatcccagtgggaccacccactacaacccgtccctcaagagtcgagtgaccatatcagtagacgcgtccaagaaccacttctccctgaacctgaactctgtgactgccgcagacacggccgtgtactactgtgccacaggatttgacaatagcaaagtgggctactggggccagggaaccctggtcaccgtctcctca

SEQ ID NO: 23 - DNA, 356 bp, Homo sapiens
aggtgcagctggtgcagtctgggactgaagtgaaaatgcctggggcctcagtgaggatttcctgcaaggcatttggatactccgtcacctcctactatatgcactgggtgcgacaggcccctggacaagggcttgagtggctgggggtaatctaccctagtggcgaccgggcaaacaccgcagagacactccagggcagagtcaccatggcgagggacacgtccacgaccaccgtctacatggaactgaccagcctgagatctgaggacgcggccgtctattactgtgctagagagccccaggagtccggactgttggactactggggccagggaaccctggtcaccgtctcctca

SEQ ID NO: 24 - DNA, 331 bp, Homo sapiens
cagtctgccctgactcagcctgcctccgtgtctgggtctcctggacagtcgatcaccatctcctgcactggaatcagaagtgatactgggaattctaaccttgtctcctggtaccagcagctcccaggcaaagtccccaaatgcatgatttttggggtcactaagcggccctcaggggtttctgatcgcttctctggcgccaagtctggcgccacggcctccctgacaatctctgggctgcaaactgaggacgaggcagattatttctgctgctcatatgcaggtcgtggcacttatgtctttggaagtgggaccaaggtcaccgtcctcg

SEQ ID NO: 25 - DNA, 346 bp, Homo sapiens
gaggtgcagctggtggagtctgggggaggcctggtccagcctggggggtccctgagactctcctgtacagcctctggattcacctttagtggctcttggatgagttgggtccgccagactccagggaaggggctggactgggtggccaccataaaggaagatggcagtaggaaagactatgtggactctgtgaagggccgatttaccatctccagagacaacgccaagaactcactgtatctgcaaatgaacagcctgagaggcgaggacacggctgtgtattactgtgcgagaggggggacttgggactccgaccactggggccagggaaccctggtcaccgtct

SEQ ID NO: 26 - DNA, 336 bp, Homo sapiens
gatattgtgatgactcagtctccactctccctgcccgtcaccctaggtcagacggcctccatctcctgcaggtcaagtcaaagcctcggacacagtaatggaaacaccttcttgtattggtttcagcagaggccaggccaatctccaagacagttaatttataaggtttctaaccgggactctggggtcccagacagattcagcggcagtgggtcaggcactgatttcacactgaaaatcagcagggtggaggctgaggatgttggggtttattactgcatgcaaggtacacactggcctccaacgttcggccaagggaccaaggtggagatcaaa

SEQ ID NO: 27 - DNA, 398 bp, Homo sapiens
aggtgcagctggtgcagtctggggctgaggtgaagaagcctggggcctcagtgaaggtttcctgcaaggcttctggatacaccttcactaactatgctatacattgggtgcgccaggcccccggacaggggcttgagtggatgggatggatcatcgttgacaatggtgacacaagatattcacaggaattccagggccgagtcacctttactagagacacatccgcgaacacagcctacatggagctgagcagcctgaaatctgaggacatggctgtgtattattgtgcgagggaccactcccgaccaggattttgtagtggtggtaactgctactcaactatttactactacagtttcgacgtctggggccaagggaccacggtcaccgtctcctca

SEQ ID NO: 28 - DNA, 375 bp, Homo sapiens
gaagtgcagctggtggagtctgggggaggcgtggtccagcctggggggtccctgagactctcctgtgcagcgtctggattcacgttcagcaattatggcatgggctgggtccgccaggctccaggcaaggggctggaatgggcggcatatatattttatgatggaagtaaaacattctatgcggactccctggagggccgaattaccatctccagagacaactccgggaacacgttgtttgtgcaattgagcagcctgagagcagacgactcggctctgtattactgtgcgagaggctcctgtgctagtaccggctgccatggttcgcgtttttttgacttctggggccagggaaccctggtcaccgtctcctca

SEQ ID NO: 29 - DNA, 363 bp, Homo sapiens; identical to SEQ ID NO: 9

SEQ ID NO: 30 - DNA, 342 bp, Homo sapiens
gacatcgtgatgacccagtctccagactccctggctgtgtctctgggcgagagggccaccatcaactgcaagtccagccagaatattttatacagctccaacaataagaactacttagcttggtaccagcagaaaccagggcagcctcctaagctgctcatttactgggcatctactcgggaatccggggtccctgaccgattcagtggcagcgggtctgggacagatttcactctcaccatcagcagcctgcaggctgaagatgtggcagtttattactgtcaacaatattatagtactcctccgtggacgttcggccaagggatcaaggtggaaatcaaa

SEQ ID NO: 31 - DNA, 380 bp, Homo sapiens
aggtgcagctggtgcagtctggagctgaggtgcagaagcctggggcctcagtgaaggtctcctgtaaggcttctggttacacctttaacaactatgatgtcagttgggtgcgacaggcccctggacaaggccttgagtggatgggatggatcagcactaacagtggtaacacaaattatgcagggaatttccagggcagagtcaccatgaccacagacacatccacgaccacggcctacatggagctggggggcctgagatatgacgacacggccgtatattactgtgggagagtttgtggctggtattgtggtagatctgcctacaaccatgactactttgactactggggccagggaaccctggtcaccgtctcctca

SEQ ID NO: 32 - DNA, 363 bp, Homo sapiens
gaagtgcagctggtggagtctgggggaggcgtggtccagcctgaagggtccctgagactctcctgtgcagcgtctggattcaccttcaagaactatggcctgcactgggtccgccaggctccaggcaagggactggagtgggtggcagttatttggtttgatggaagtaataaaatatatgcagattctgtgaagggccgattcaccgtctccagggacgattccaaaaacacgctgtttctgcaaatggacagcctgagagtcgaggacacggctatatattactgtgcgagagaggattgtcctagtggattctgcacttctgactactggggccagggaaccctggtcaccgtctcctca

SEQ ID NO: 33 - DNA, 372 bp, Homo sapiens
gaggtgcagctggtggagtctgggggaggcttggtaaagcctggggggtcccttagactctcctgtgcagcctctggattcactttcagtagcgcctggttgagctgggtccgtcaggatccagggaaggggctggagtggcttggccgtattaaaagtaaagctgctggtgggacagcagaccacgctgcatccgtgaaaggcagattcaccatctcaagagatgattcaaaaaacacgttatatctgcaaatgaacagcctgaaaatcgaggacacagccgtctattactgtaccacagggtcattggagggtacaattacacacgcggaggaccactggggccagggaaccctggtcaccgtctcctca

SEQ ID NO: 34 - DNA, 321 bp, Homo sapiens
gacatccagatgacccagtctccatcctccctgtctgcatctgtgggggacagagtcaccatcacttgccgggcaagtcagagcgttagcaggtatctaaattggtatcagcagaaaccagggagagcccctaggctcctgatctttggtgcatccagtttacgtagtggagtcccatccaggttcagtggcagtggatctgggacagatttctctctcaccatcaacagtctgcaacctgaagattttgcaacttactactgtcaacagacttacagtactttcggacctttcggccctgggaccaaagtggatatcaaa

SEQ ID NO: 35 - DNA, 369 bp, Homo sapiens
gaggtgcagctggtggagtctgggggaggcctggtcaagccgggggggtccctgagactctcctgtgcagcctctggattcaccttcagtagctataccatgatctgggtccgccaggctccagggaaggggctggagtgggtctcatccattactagtagtagtagtcacatatactacgcagactcagtgaagggccgattcaccatctccagagacaacgccaagaactcactgtatctgcaaatggacagtctgagagccgaggacacggctgtgtattactgtgcgagagtaaacggcgaactgggaccgacagaatactactttgactactggggccagggaaccctggtcaccgtctcctca

SEQ ID NO: 36 - DNA, 351 bp, Homo sapiens
gaggtgcagctggtggagtctgggggaggcttggtccagcctggggggtccctgagactctcctgtgcagcctctggattcacctttagtagctactggatgagctgggtccgccagactccagggaaggggctggagtgggtggcccacataaagcaagatggaagtgagagacactatgtggactctgtgaaggaccgattcaccatctccagagacaacgccaagaactcactgtatctgcaaattaacagcctgagagccgaggacacggctgtttattactgtgcgagaggagggacttgggatgcctactactggggccagggaaccctggtcaccgtctcctca

SEQ ID NO: 37 - DNA, 333 bp, Homo sapiens
gatattgtgatgacccagactccactctctctgtccgtcacccctggacagccggcctccatctcctgcaagtctagtcagagcctcctgcatagtaatggaaagacctatttgtattggtacctgcagaagccaggccagtctccacagctcctaatctatgaagtttccagccggttctctggagtgccagataggttcagtggcagcgggtcagggacagatttcacactgaaaatcagccgggtggaggctgaggatgttggggtttattactgcatgcaaggtgtacaccttcccactttcggcggagggaccaaggtggagatcaaa

SEQ ID NO: 38 - DNA, 359 bp, Homo sapiens
caggtgcagctacagcagtggggcgcaggactgctgaagccttcggagaccctgtccctcacctgcgctcttcataatggattcttcaatacttactactggggctggttccgccagcccccagggaaggggccggagtggattggggaaatcaatcatagtggaaacaccaactacgacccgtccctcaaaagtcgagccaccatatcaattgacacgtccaagaagcacttctccctgaacctgacctctcttaccgccgcggacacgggtgtgtatttttgtgcgaggttgcctcagcgatattactttggttcggggagttccaactggggccagggaaccctggtcaccgtctc

SEQ ID NO: 39 - DNA, 325 bp, Homo sapiens
gaaattgtgttgacgcagtctccaggcaccctgtctttgtctccaggggagagagccaccctctcctgcggggccagtcagactgttagcgacaactatttagcctggtaccagcagaaacctggccaggctcccaggctcctcatctttggtgcatccaacagggccactgacgtcccagacagattctatggcagtgggtctgggacagacttcactctcaccatcagcagactggagcctgaagattttacattttattactgtcagcactatggtagctcaccttacacttttggccaggggaccaaggtggagatcaaac

SEQ ID NO: 40 - DNA, 349 bp, Homo sapiens
gaggtgcagctggtggagtctgggggaagcctggtcaggactggggggtccctgagactctcctgtgcagcctctggattcaccttcagtagttatagcatgaactgggtccgccaggctccagggaaggggctggagtgggtctcatccattagcagtactggtaattccatatactacgcagactcagtgaagggccgattcaccatgtccagagacaacgccaagaactcactgtatctgcaaatgaacagcctgagagccgaggacacggctgtgtattactgtgtttacggctggtacctctttgactattggggccagggaaccctggtcaccgtctcctcag

SEQ ID NO: 41 - DNA, 337 bp, Homo sapiens
gatattgtgatgactcagtctccactctccctgcccgtcacccttggacagccggcctccatctcctgcaggtctagtcaaagcctcgtacacagtgatggaaacacctacttgaattggtttcagcagaggccaggccaatctccaaggcgcctaatttataaggtttctaagcggggctctggggtcccagacagattcagcggcagtgggtcaggcactgatttcacactgcaaatcagcagggtggaggctgaggatgttgggatttattactgcatgcaaggtacacactggcctcggacgttcggccaagggaccaaggtggagatcaaac

SEQ ID NO: 42 - DNA, 347 bp, Homo sapiens
gaggtgcagctggtggagtctgggggaggcttggtccagcctggggggtccctgagactctcctgtgtagcctctggattcacttttagtgcccattacatcagctgggtccgccaggctccagggaaggggctggagtgggtgaccaacacaccgcgcgatggaagacagacacactatgtggactctgtgaacggccgattcaccgtctccagagacaacaccaagaacacactgtatttgcaaatgagcagcctgagagtcgaggacacggctgtctattattgtgcgagagacgacctcagcgtccaccagtactggggcctgggaaccctggtcaccgtctc

SEQ ID NO: 43 - DNA, 322 bp, Homo sapiens
gaaatagtgatgacgcagtctccagacaccctgtctgtgtctccaggggatagcgccaccctctcctgcagggccagtcagagtcttaccagcaggctggcctggtaccagcagaaacctggccagcctcccaggctcctcctctatggtgcatccaccagggccactggtgttccagccaggttcagtggcagtgggtctgggacagagttcactctcaccatcagcagcctgcagtctgaagattttgcaatttatcactgtcagcagtatgattcctggccttggacgttcggccaagggaccaaggtggaaatcaaac

SEQ ID NO: 44 - DNA, 370 bp, Homo sapiens
aggtgcagctggtggagtctgggggaggctcggtcaagcctggagggtccctgagactctcctgcgcagcctctggattcgcctttggtgactactatatgagctggatccggcaggttccagggaagggcctgcagtggatttcatacataagtcctactagtatttacacaaactatgcagactctgtgaagggccgattcaccatctccagagacaacgtcaggaatgcgctgtatctggaaatgagcaggctgagtgccgaagatacggctgtctatttttgtgtgagagattggtgggaggtcaaggatagcaacaactggttctgggcggaccactggggccagggaaccctggtcaccgtctc

SEQ ID NO: 45 - DNA, 321 bp, Homo sapiens
tcctatgagctgacacagccaccctcggtgtcagtgtccccaggacagacggccagaatcacctgctctggagatgcattgccgagacactatgccttttggtaccaacaaaaggcaggccaggcccctgttttggtgatatttaaagacattcagaggccgtcagaaatccctgcgcgattttctggctccagttcagggacaacagtcacgttgaccatcagtggagtccagccagaagacgaggctgactattactgtcaatcagtggacaacagtggtctttatcgtgtgttcggcggagggaccaaattgaccgtc

SEQ ID NO: 46 - DNA, 358 bp, Homo sapiens
gaggtgcagctggtggagtctgggggagacttggtccagcctggggggtccctgagactctcctgtgcagcctctggactcatctttaatcagtattggatggcatgggtccgccaggctccagggaaggggctggagtgggtggcccacataaagtcggatgcaactctacaagtctatgtggactctgtgaagggccgattcaccatctccagagacaacgccaaggactctctgtttctgcaaatgaacagcctgagagacgaggacacggccgtctactactgtgcgagagattcgtatggttgggggagttatgactactggggccacggaaccctggtcaccgtctcctcag

SEQ ID NO: 47 - DNA, 334 bp, Homo sapiens
cagtctgccctgactcagcctcgctcagtgtccgcgtctcctggacagtcagtcgccatctcctgcactggaaccagcagtgatgttggtggttataactctgtctcctggtaccaacagcacccaggcaaagcccccaaactcatgatttatactgtcagtaagcggccctcaggggtccctgatcgcttctctggctccaagtctggcaacacggcctccctgaccatctctgggctccaggctgaggatgaggctgattattactgctgctcattttcaggcactgacggttcttatgtcttcggaagtgggtccaaggtcaccgtcctag

SEQ ID NO: 48 - DNA, 387 bp, Homo sapiens
aggtgcagctggtggagtctgggggaggcttggtcaagcctggagggtccctgagactctcctgtgcagcctctggattcaccttcagtgactacgacatgaggtggatccgccaggctccagggaaggggctggagtgggttgcatatgttagtggtagtcttggtcacacaaatagtcacacaaagtacgcagactctgtgaagggccgattcaccatctccagagacgacgccaagaagtcactgtctttgcaaatgaacagcctgagagtcgacgacacggctgtgtatttctgtgcgacagaaaattcgggagggtatcggagtggctggtggcgagtgtttgacaactggggccacgggacgctggtcaccgtctcctcag

SEQ ID NO: 49 - DNA, 331 bp, Homo sapiens
cagtctgtgctgacgcagcctcccgcagcgtcggggacccccggacagagggtcaccatctcttgttctggaagcagctccaacatcggaagtaatactgtaaactggtaccagcagctcccgggaacggcccccaaactcctcatctacggtcttgatcagcggccctcaggggtccctgaccgattctctggctccaagtctggcacctcagcctccctggccatcagtgggctccagtctgaggatgaggctgattattactgtgcaacatgggatgacggcctgaatggtccggtgttcggcggagggaccaagctgaccgtcctag

SEQ ID NO: 50 - DNA, 350 bp, Homo sapiens
caggtgcagctgcaggagtcgggcccaggactggtggagccttcacagaccctgtccctcacctgcagtgtctctggtggctccatcagtagtagtggttacttctggagctggatccgccagcacccagggaagggcctggagtggattgggtgcatcaccaacagtgggagtattacctacaacccgtccctcgggagtcgaattacgatatcaccggacacgtctaagaaccagttctccctgaaactgaactctgtgactgtcgcggacacggccgtctattactgcgcgagaatggggaatttccaaggagtctattggggccagggaaccctggtcaccgtctc

SEQ ID NO: 51 - DNA, 334 bp, Homo sapiens
cagactgtggtgactcaggagccctcactgactgtgtccccaggagggacagtcactctcacctgtggctccagcactggagcagtcaccagtggtcattatccctactggttccagcagaagcctggccaagcccccaggacactgatttatgatacagactacaaacactcctggacacctgcccggttctcaggctccctccttgggggcaaaggtgccctgaccctttcgggtgcgcagcctgaggatgaggctgagtattactgcttgctctcctacaatagtggtcggctttgggtgttcggcggagggaccaaactgaccgtcttag

SEQ ID NO: 52 - DNA, 352 bp, Homo sapiens
aggtgcagctggtgcagtctggggctgaggtgaagaagcctggggcctcagtgaaagtctcctgcaaggcttctggatacaccttcagcggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagttgagcaggctgagatctgacgacacggccgtttactactgtgcgagagatttttatagccaagggtggtacgactactggggccagggaaccctggtcaccgtctc

SEQ ID NO: 53 - DNA, 360 bp, Homo sapiens
aggtgcagctggtgcagcctggggctgacgtgaagaagcctggggcctcagttaggctttcctgcgccgcctctggatacaccttcaccggttatactacccactggctgcgccaggcccccggacaagcgcttgagtggatggggtggatacacggtgacagtggtgacacaaaatattcacagaagtttcaagatagggtcactatcacccaggactcatccgcgagcacagcctacatggaaatgaccaggctgcgacacgacgacacggctgtctactactgtgcgagaggctttagcagcacctcatattggggtcgcttcgacccctggggccagggaaccctggtcaccgtct

SEQ ID NO: 54 - DNA, 360 bp, Homo sapiens
gtgcagctggtgcagtctggagcagaggtgaaaaagcccggggagtctctgaagatctcctgtaagggttctggatacagctttagcaactactggatcggctgggtgcgccagatgcccgggaaaggcctggagtgggtggcgatcatctatcctggtgactctgataccagatatagtccgtccttccaaggccaggtcaccatctcagccgacaagtccatcagcaccgcctacctgcagtggaacagcctgaaggcctcggacaccgccaggtattactgtgggagacatggagcagtaattccaactaccaaatactatcacttctacggtatggacgtctggggccaagggacc

SEQ ID NO: 55 - DNA, 331 bp, Homo sapiens
cagtctgtgctgacgcagccgccctcagtgtctgcggccccaggacagaaggtcaccatctcctgctctggaagcagctccaacattgggaataactatgtttcctggtaccagcagctcccaggcacagtccccaaactcctcatttatgacaatgataagcgaccctcagggattcctgaccgattctctggctccaagtctggcacgtcagccaccctgggcatcaccggactccagactggggacgaggccgattattactgcggaacatgggatagcagtctgcgtgctggggtattcggcggagggaccaaggtgaccgtcctag

SEQ ID NO: 56 - DNA, 398 bp, Homo sapiens
gaggtgcagctggtggagtccgggggaggtttggtacagccagggcggtccctgagactctcctgtacaacctctggattcacctttggagattatgttatgagttggttccgccaggctccagggaaggggctggagtgggtaggtttcattagaaggagagcttatggtgggacagcagagtatgccgcgtctgtgaaaggcagattcagcatctcaagagatgattccaaaagcatcgcctttctgcaaatgaacagcctgagaaatgaagacacaggcgtctattattgtactagagagggagacgtatcagtgtatttggagtggtcaactggaggaccctacatttacaacggtttggacgtctggggccaagggaccacggtcaccgtctc

SEQ ID NO: 57 - DNA, 319 bp, Homo sapiens
gaaatagtgatgacgcagtctccagccaccctgtctgtgtctccaggggaaagtgccaccctctcctgcagggccagtcagagtgttggccgcaacttagcctggtaccagcagaagctaggccaggctcccaggctcctcatctatgatacatccaccagggccactggtgtcccagccaggttccgtggcagtgggtctgggacagagttcactctcaccatcagcagcctgcagtctgaagattttgcagtttattactgtcagcagtataataactggcggacgttcggccaagggaccaaggtggaaatcaaac

SEQ ID NO: 58 - DNA, 377 bp, Homo sapiens
gaggtgcagctgttggagtctgggggaggcttggtacagccgggggggtccctgagactctcctgtgcagcctctggattcacctttagcagctatgccatgagctgggtccgccaggctccagggaaggggctggagtgggtctcagctattagtggtggtggtggtagcacatactacgcagactccgtgaagggccggttcaccatctccagggacaattccaagaacactctgtatctgcaaatgaacagcctgagagccgaggacacggccgtatattactgtgcgaaacgatattgtagtagtaccagctgccatctttactactactacggcatggacgtctggggccaagggacccggtcaccgtctcg

SEQ ID NO: 59 - DNA, 329 bp, Homo sapiens
cagtctgccctgactcagcctccctccgcgtccgggtctcctggacagtcagtcaccatctcctgcactggaaccagcagtgacgttggtgcttataactatgtctcctggtaccaacaacacccaggcaaagcccccaaactcatgatttatgaggtcagtaagcggccctcaggggtccctgatcgcttctctggctccaagtctggcaacacggcctccctgaccgtctctgggctccaggctgaggatgagactgattattactgcagctcatatgcgggcagcaacaatttggtattcggcggagggaccaagctgaccgtcct

SEQ ID NO: 60 - DNA, 356 bp, Homo sapiens
caggtgcagctacagcagtggggcgcaggactgttgaagccttcggagaccctgtccctcacctgcgctgtctatggtgggtccttcagttattactactggagctggatccgccagcccccagggaaggggctggagtggattggggaaatcaatcatagtggaagcaccaactacaacccgtccctcaagagtcgagtcaccatatcagtagacacgtccaagaatcagttctccctgaagctgagctctgtgaccgccgcggacacggctgtgtattactgtgcgacgaagaatttctatggttcggggagttgggtcgtggtctggggccaagggaccacggtcaccgtctc

SEQ ID NO: 61 - DNA, 325 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcaccagactggagcctgaagattttgcagtgtattactgtcagcagtatggtagctcctcgtacacttttggccaggggaccaaggtggaaatcaaac

SEQ ID NO: 62 - DNA, 365 bp, Homo sapiens
caggtgcagctggtgcagtctggggctgaggtgaagaagcctggggccccagtgaaggtttcctgcaaggcatctggatacaccttcaccaactactatatgcactgggtgcgacaggcccctggacaaggacttgagtggctgggactaatcaacgctggttctggtaggacaaggtacgcaccgaagttccagggcagagtcaccatgaccagggacacgtccacgagcacactctacatagaggtggacagcctgagatccgacgacacggccgtttattactgtgcgagaggatccgcccatatagaagcgggtggttccgcctttgacaagtggggccagggaaccctggtcaccgtctc

SEQ ID NO: 63 - DNA, 337 bp, Homo sapiens
gatattgtgatgactcagtctccactctccctgcccgtcacccttggacagccggcctccatctcctgcaggtctagccaaagcctcgcatacagtgatggaaacacctacctgaattggtttcaccagaggccaggccactctccaaggcgcctaatttataaggtttttaatcgggactctggggtcccagacagattcagcggcagtgggtcaggcactgatttcacactgaaaatcagcagggtggaggctgaggatgttggggtttattactgcatgcaaggtacacactggccgatcaccttcggccaagggacacgactggagattaaac

SEQ ID NO: 64 - DNA, 365 bp, Homo sapiens
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagaggatacagctatggctcccggatttgactactggggccagggaaccctggtcaccgtctc

SEQ ID NO: 65 - DNA, 331 bp, Homo sapiens
cagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttatgtcttcggaactgggaccaaggtcaccgtcctag

SEQ ID NO: 66 - DNA, 365 bp, Homo sapiens
caggtgcagctggtgcagtctggggctgaggtgaagaagcctggggccccagtgaaggtttcctgccaggcatctggattcattttcatcaattactatttccactgggtgcggcaggcccctggacaaggcctggagtggatgggagtaataaaccccactagtggtaagtcaaaactcgcgccgacgttggagaatcgagtcaccatgaccagcgacacgtccacgagaacactttacatagaggtgaccagcctgacatctgacgacacggccatatattactgtgcgagagggtccgcccgcgcagaagcggctggttccgcctttgaccactggggccagggaaccctggtcaccgtctc

SEQ ID NO: 67 - DNA, 337 bp, Homo sapiens
gatattgtgatgactcagtctccactgtccctgcccgtcatccttggacagtcggcctccatctcctgcaggtctagtcaaagcctcatacacagtgatgggaacatcttcttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggcgtttattactgcatgcaaggtacacactggccgatcaccttcggccaagggacacgactggagattaaac

SEQ ID NO: 68 - DNA, 374 bp, Homo sapiens
caggtgcagctgcaggagtcgggcccaggactggtgaagccttcggagaccctgtccctcacctgcactgtctctggtacctccatcaggaattctaattattactgggcctggatccgccagcccccagggaagggactggagtggattggaaccgtcttctatagtgggagttcctactccaacccgtccctcaagggtcgagtcaccatatctgtagacacgtccaagagccagttctccctgaagttgcccgctgtgaccgccgcggacacggccgtgtatttctgtgcgggctccccgtataactgggactattcttctcaatacaatcggttccatccctggggccagggaaccctggtcaccgtctc

SEQ ID NO: 69 - DNA, 319 bp, Homo sapiens
gaaattgtgttgacgcagtctccagacatcctgtctttgtctccaggggatagagcctccctctcctgcagggccagtgacaatattgacagcgacttagcctggtaccagcagaaacctggccaggctccccggctcctcatctttggtgcttccaggagggccactggcatcccagacaggttcagtggcagggggtctgggacagacttcactctcagcatcagcagactggaggctgaagattttgcagtgtattactgtcagcagtatggtacttcgcccactttcggcggagggaccaaggtggaaatcaaac

SEQ ID NO: 70 - DNA, 356 bp, Homo sapiens
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagtgttgatagcagcggctggtactactttgactactggggccagggaaccctggtcaccgtctc

SEQ ID NO: 71 - DNA, 328 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctccgtacacctttggccaggggaccaaggtggaaatcaaac

SEQ ID NO: 72 - DNA, 383 bp, Homo sapiens
gaggtgcagctgttggagtctgggggaggcttggtacagcctggggggtccctgagactctcctgtgcagcctctggattcaactttggtacctatgccgtgagctgggtccgccaggcccccgggaaggggctggagtggatctcaggcattagtgatagtggtggtgacacatactacgcagactccgtgaagggccggttcaccatctccagagacaattccaatagcacactgtatctgcaaatgaacagcctgagagccggggacacggccgtttatcactgtgcgaagagcccggtgtcttactatggttcggggaatttttattcgaactactactactttgacaactggggccagggaaccctggtcaccgtctc

SEQ ID NO: 73 - DNA, 322 bp, Homo sapiens
gacatccagatgacccagtctccatcctccctgtctgcatctgtaggagacagagtcaccatcacttgccgggcaagtcagagtctgaatacttatttaaattggtatcagaaacaaccagggaaagcccctaaactcctgatctatgctgcgtccacattgcagcgtggggtcccatcaaggttcagtggcagtggatctgggacagatttcactctcaccatcagcagtctgcaacctgcggattttgcaacttactactgtcaacagagtcacagtccgccattcactttcggccctgggaccaaagtggatatcaaac

SEQ ID NO: 74 - DNA, 383 bp, Homo sapiens
gaggtgcagctgttggagtctgggggaggcttggtaaagcctggggggtccctgagactctcctgtgcagcctctggactcaccttcagcacctttgccatgagctgggtccgccagcctccagggaaggggctggagtgggtctcaggaattagtgatagtggtggtaacacatattacgcagactccgtgaggggccggttcaccatctccagagacaattccaagaacaccctgtatctgcaaatcaacagcctgagagccgaggacacggccgtattttactgtgcgaaaagtccggtgtcttactatggttcggcgagtttttattcgaactactattactttgactcctggggccagggaaccctggtcaccgtctc

SEQ ID NO: 75 - DNA, 322 bp, Homo sapiens
gacatccagatgacccagtctccattctccctgtctgcatctgtaggagacagagtcaccatcacttgccgggccagtcagaccattagcagctatttaagttggtatcagcagaaaccagggagagcccctaaactcctaatctatgcttcatccactttgcaaactggggtcccatcacggttcagtggcagtggatctgggacagatttcactctcaccatcagcagtctggaacctgaggattttgcggcttactactgtcaacagacttccagtcccccattcactttcggccttgggaccaaagtggatatcaaac

SEQ ID NO: 76 - DNA, 374 bp, Homo sapiens
caggtgcagctgcaggagtcgggcccaggactggtgaagccttcggagaccctgtccctcacctgcactgtctctggtgcctccatgaatggttactactggaactggatccggcagcccccagggaagggactggagtggattggacatatccattacgttgggactaccatgaaggccaccaacgacagcccctccctcaggagtcgagtcaccatatcagtggacacgtccaggagccagatctccctgaagctgacgtctgtgaccgccgcggacacggccgtgtattactgtgcgagaaggcggtaccatttttggagtgaccccggaacgtttgatatctggggccaagggacaatggtcaccgtctc

SEQ ID NO: 77 - DNA, 322 bp, Homo sapiens
gacatccagatgacccagtctccatcctccctgtctgcatctgtaggagacagagtcaccatcacttgccaggcgagtctggacattaaaaactatctaaattggtatcagcagaaaccaggggaagcccccaagctcctgatctccgatgcatccaccttggaaaccggggtcccatcaaggttcagtggaagggggtctgggacacatttcactgtcaccatccgcagcctgcagtctgaagatgttgcaacatattactgtcaacaatatggtaaactcccattcactttcggccctgggaccaaagtggatatcaaac

SEQ ID NO: 78 - DNA, 371 bp, Homo sapiens
caggtgcagctgcaggagtcgggcccaggactggtgaagccttcggggaccctgtcactcacttgcagtgtctctggtgtctccataagtagtagatattggtggaattgggtccgccagtccccggggaaggggctggagtggattggagaagtctaccatagtgggagttccaactacaacccgtctctcaagagtcgagccaccatttcagcagacaagtccaacaaccagttctccctgacactgacctctgtgaccgccgcggacacggccctctattactgtgtgagagatcgattaagtgaagacggttatggctccgcgggttatttcgacccctggggccagggaaccctggtcaccgtctc

SEQ ID NO: 79 - DNA, 322 bp, Homo sapiens
gacatccagatgacccagtctccatcctccctgtctgcatcggtgggagacagagtcaccatcacttgccgggcgagtcaggtcattagaaatgatttagcctggtatcagcagaaaccagggaaagcccctaagcgcctgatctatggtgctttcagtttgcaaagtggggtcccttctaggttcagcggcagtagatctgggacagaattcactctcacaatcagcggcctgcagcctgaagattttgcaacttattactgtctacagtataatggttaccctcggacgttcggccaagggaccaaggtggaaatcaaac

SEQ ID NO: 80 - DNA, 368 bp, Homo sapiens
caggtgcagctgcaggagtcgggcccaggactggtgaagccttcggagaccctgtccctcacgtgctctgtctctggtggctccatcaatggttattactggaactggatccggcagtccccagggaaggggctggagtggattggtcatatctataaccttgcgaccactaccaccgaggccaatccctccctcaggagtcgactcgccatctccatagacacgtccaagagccaggtctccctggaactgaattctgtgaccgctgcggacacggccgtctattattgtgcgagaaggaagtatcatttttggagtgaccccggtgcatttgatgtctggggccaagggacaatggtcaccgtctc

SEQ ID NO: 81 - DNA, 322 bp, Homo sapiens
gacatccagatgacccagtctccatcctccctgtctgcatctgtacgagacagagtcaccatcacttgccaggcgagccaggacattactaagtctttaaattggtatcaacagaaaccaggaaaagcccctaagctcctgatttccgatgcatccaaattggaaactggggtcccgtcgaggttcagtggcggtggatctgggacagattttactttcaccatcagcagcctgcagcccgaagatattgcttcatattactgtcaacaatatggtgatctcccattcactttcggccctgggaccaaagtggatatcaaac

SEQ ID NO: 82 - DNA, 374 bp, Homo sapiens
caggtgcagctgcaggagtcgggcccaggactggtgaaggcctcggagaccctgtccctcacctgcgctgtctctggtgtctccatcagaagtgctaatcactactgggcctgggtccgccagcccccagggaaggggctggagtggattgggactatcttttatagtgggaccacctactacaacccgtccgtcaagagtcgagtcaccatgtcagtcgacacgtccaagaaccagttctccctgaaggtaacgtctgtgaccgccgcggacacggccgtatattactgtgcgagcagcccctataactgggactacgcttctcagtacaatcgactcgacccctggggccagggaaccctggtcaccgtctc

SEQ ID NO: 83 - DNA, 322 bp, Homo sapiens
gaaattgtgttgacgcagtctccaggcaccctgtctttgtctccaggggaaagagccaccctctcctgcagggccagtcagagtcttagcagcacctacttagcctggtatcagcagaaacctggccaggctcccaggctcctcctctatgctgcatccagaagggccactggcatcccagacaggttcagtggcagtgggtctgggacagacttcactctcaccatcagcagactggaccctgaagattttgcagtgtactactgtcagcagtatgctgcctcgctcactttcggcggagggaccaaggtggagatcaaac

SEQ ID NO: 84 - DNA, 362 bp, Homo sapiens
gaggtgcagctggtggagtctgggggaggcctggtcaagcctggggggtccctgagactctcctgtgcagcctctggattcaccttcaggagctataccatgaactgggtccgccaggctccagggaaggggctggagtggctctcaaccattagtgatagtggtaacttcatatattatgcagcctcagtgaggggccgattcaccatctccagagacaacgccaagaactcactgtatctgcaaatgaatagcctgagagtcgaggacacggctgtttattactgtgcgagagatttttggcagctcgtcggcgggaggggatttgactactggggccagggaaccctggtcaccgtctc

SEQ ID NO: 85 - DNA, 349 bp, Homo sapiens
cagcttgtgctgactcagccaccttctgcatcagcctccctgggagcctcggtcacactcacctgcaccctgagtagcggctacagtaattataaggtggactggtaccaacagagaccagggaagggcccccgctttgtgatgcgagtgggcactggtggaattgtgggatccaagggggatggcatccctgatcgcttctcagtcatgggctcaggcctgaatcggtacctgaccatcaagaacatccaggaagaggatgagagtgactaccactgtggggcagagcatggcagtgggaccaacttcgtgtgggtgttcggcggagggatcaagctgaccgtcctag

SEQ ID NO: 86 - DNA, 367 bp, Homo sapiens
gaggtgcagctggtggagtccgggggagacttggtccagcctggggggtccctgaaactctcgtgtgcagcctctggcttcacattcagtgtctctgctatgcactgggtccgccaggcttccgggaaaggcctggagtggcttggccgcattagaggcaaagctaataattacgcgacatcatatgctgcgtcggtgaaaggcaggttcaccatctccagagatgactcaaagaatacgacctatctgcaaatgaacagcctgaaaatcgaggacacggccgtgtattactgtcaccttgcgcgacactatgatagtaatgattatcccattgacagctggggccagggaaccctggtcaccgtct

SEQ ID NO: 87 - DNA, 325 bp, Homo sapiens
tcctatgagctgacacagccaccctcggtgtcagtgtccccaggacagacggccaggatctcctgctctggagatgcattggcaaacgaatatacttattggtatcggaaaaagccaggccaggcccctgttctggtgatatatagagacactgagaggccctcagggatccccgagcgattctctggctccggctcagggacgacagtcacattgaccatcactggagtccaggcagaagacgaggctgactattactgtcaatcagtcgacaacagtaatgatctttgggtgttcggcggagggaccaagctgaccgtcctag

SEQ ID NO: 88 - DNA, 358 bp, Homo sapiens
gaggtgcagctggtggagtctgggggaggcttgatagagcctggggggtcccttagactctcctgtgaagcctctggattccttttcagtaacacctggatgacttgggtccgccaggctccagggaagggactggagtgggttggccatattaaagacactgctgatggtgggacagcagactacgctgcacccgttaaaggcagattcaccatttcaagagatgattcgcagaacgcgctctttctacagatgaacagtctgaaacccgaggactcaggcgtttattactgtagcaccctgtacggcggtaaccaggccataggctactggggccagggaaccctggtcaccgtct

SEQ ID NO: 89 - DNA, 337 bp, Homo sapiens
tcctatgagctgactcagccaccctcggtgtcagtgtccccaggacaaacggccaggatcacctgctctggagaggcattgccaaaaagatatacttattggtaccagcagaagtcaggccaggcccctctgctgatcatctatgaggacaacaaacgaccttcccgcaaacgaccctccgagatccctgagagattctctggctccagctcagggacaatggccaccttgactatcagtggggcccaggtggaggatgaggctgactactattgttactcaacggacaccagtggtcatccccaattcggcggagggaccaaactgaccgtcctag

SEQ ID NO: 90 - DNA, 364 bp, Homo sapiens
caggtacagctgcagcagtcaggtccaggactggtgaagccctcggagaccctctcactcacctgtgtcatctccggggactttgtctctagcaacagtgctgcttggaactggatcaggcagtccccatcgagaggccttgagtggctgggaaggacctactacaggtccaagtggtataatgaatatgccgcatctgtgaaaagtcgaataaccatcaacccagacacatccaagaaccagttctccctccagctgaactctgtgactcccgaggacacggctgtctattactgtgtaagagatggtgatagtggccccgaattctttgactactggggccagggaaccctggtcaccgtct

SEQ ID NO: 91 - DNA, 331 bp, Homo sapiens
cagtctgtgctgactcagccgccctcagtgtctgcggccccaggacagaaggtcaccatctcctgctctggaagcagctccaacattgggaagaactctgtggcctggcaccagcaactcccaggaacagcccccaaactcctcatctatgagaatgataagcgaccctcagggattcctgaccgattctctgcctccaagtctgacacgtcagccaccctgggcatcaccggactccagactggggacgaggccgattattactgcgcaacatgggacagcccgagtaagtcctatgtcttcggaactgggaccaaggtcaccgtcctag

SEQ ID NO: 92 - DNA, 367 bp, Homo sapiens
gaggtgcagctggtggggtccgggggaggcttggtccagcctggggggtccctgaaactctcctgtgcagccgctggatttactttcagtgccgctgctatgcactgggtccgccaggcttccgggaaagggctggagtgggttggccgtattagagacaaaggtaacaattacgcgacagcatatgctgagtcgcttaaaggcagattcaccatttccagagatgattcaaagaacacggcttatttggaaatgaacaacctgaaaaccgaggacacggccatttattactgtcaccttgcgcggcattatgatgggagtgattttccccttgactactggggccagggaaccctggtcaccgtct

SEQ ID NO: 93 - DNA, 328 bp, Homo sapiens
tcctatgagctgacacagccaccctcggtgtcagtgtccccaggacagacggccacgatcacctgctctggagatgcattgcccaaggaatttgcttactggtaccagcagaagccaggccaggcccctatattaattatctataaggacgttgagaggccttcagggatccctgagcgattctctggctccacctcagggacaacagtcacgttgaccatcagtggagtccaggcagaggacgaggctgactattattgtcaatcagcagacaacagtggtccttatgtttgggtgttcggcggaggcaccaaactgaccgtcctaa

SEQ ID NO: 94 - DNA, 351 bp, Homo sapiens
gaggtgcagctggtgcagtccggagcagaggtgaaaaagcccggggagtctctgaggatctcctgtaagggttctgaatatagcttcaccacctactggatcacctgggtgcgccagatgcccgggaaaggcctggagtggatggggaggattgatcctagtgactcttacaccaattacagcccgtacttccaaggccacgtcactatctcagttgacaagtccatcagtactgcctacctgcagtggagcagcctggaagcctcggacaccgccatgtattactgtgcgagactgcggggatacgactttgaccactggggccagggaaccctggtcaccgtctcctca

SEQ ID NO: 95 - DNA, 331 bp, Homo sapiens
cagtctgccctgactcagcctgcctccgtgtctgggtctcctggacagtcgatcaccatctcctgcactggaaccagcagtgacgttggtggttttatctatgtctcctggtaccaacaactcccaggcgaagcccccagaatgatgatttatgatgtcagtaatcggccctcaggggtttctcatcgcttctctggctccaagtctggcaacacggcctacctgagtatctctgggctccaggctgaggacgagggtgattattattgctgctcttatcgaagcagcagcactctggtgttcggcggagggaccaagctgaccgtcctag

SEQ ID NO: 96 - DNA, 347 bp, Homo sapiens
aggtgcagctggtgcagtctggagctgaggtgaagaagtctggggcctcagtgagggtctcctgcaagacctctggttacacctttaccagttacggtatcacctgggtgcggcaggcccctggacaagggcttgagtggatgggatggatcatcccttacaatggtaacacacagtacgcacagaagttccagggcagagtcaccatgacaacagacacatccacgaccacagcctacatggacctgaggagcctgagatctgacgacacggccgtatattactgtgcgagagaagttgacgggtttgactactggggccagggaaccctggtcaccgtctcctca

SEQ ID NO: 97 - DNA, 331 bp, Homo sapiens
cagactgtggtgacccaggagccatcgttctcagtgtcccctggaggggcagtcacactcacttgtggcttgagctctgactcagtgtctactgcctaccaccccagctggtaccagcagaccccaggccaggctccacgcacgctcatctacagcacaaatgttcgctcttctggggtccctgatcgcttctctggctccatccttgggaacaaagctgccctcaccatcacgggggcccaggcagatgatgactgtgactattactgtgggctgtatatgggtaatgccatttgggtattcggcggagggaccaagctgaccgtcctag

SEQ ID NO: 98 - DNA, 378 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagactggggggtacagtaactcctcttcggtactactactactacatggacgtctggggcaaagggaccacggtcaccgtctcctca

SEQ ID NO: 99 - DNA, 331 bp, Homo sapiens
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactctggtgttcggcggagggaccaagctgaccgtcctag

SEQ ID NO: 100 - DNA, 362 bp, Homo sapiens
aggtgcagctggtgcagtctggggctgaggtgaagaagcctggggcctcagtgaaggtttcctgcaaggcctctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatctgaagacacggctgtgtattactgtgcgagtgattgtagtagtaccagctgctatgcttttgactactggggccagggaaccctggtcaccgtctcctca

SEQ ID NO: 101 - DNA, 327 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctaggtacacttttggccaggggaccaaggtggaaatcaaa

SEQ ID NO: 102 - DNA, 364 bp, Homo sapiens
gaggtgcagctggtggagtccgggggaggcttagttcagcctggggggtccctgagactctcctgtgcagcctctggattcaccttcagtagctactggatgtactgggtccgccaagctccagggaaggggctggtgtgggtctcacgtattaatagtgatgggagtaccacgaactacgcggactccgtgaagggccgattcaccatctccagagacaacgccaagaacacgctgtatctgcaaatgaacagtctgagagccgaggacacggctgtgtatttctgtgcaagatatgcccacctggggataggttggtacttcgatctctggggccgtggcaccctggtcaccgtctcctcag

SEQ ID NO: 103 - DNA, 331 bp, Homo sapiens
cagtctgccctgactcagcctcgctcagtgtccgggtctcctggacagtcagtcaccatctcctgcactggaaccagcagtgatgttggtggttataactatgtctcctggtaccaacagcacccaggcaaagcccccaaactcatgatttatgatgtcagtaagcggccctcaggggtccctgatcgcttctctggctccaagtctggcaacacggcctccctgaccatctctgggctccaggctgaggatgaggctgattattactgctgctcatatgcaggcagctacactttgttattcggcggagggaccaagctgaccgtcctag

SEQ ID NO: 104 - DNA, 369 bp, Homo sapiens
gaggtgcagctggtgcagtctggagcagaggtgaaaaagcccggggagtctctgaagatctcctgtaagggttctggagacagttttacgagttactggatcggctgggtgcgccagatgcccgggaaaggcctggagtggatggggatcatctctcttggtgactctgataccagatacagaccgtccttccaaggccaggtcaccatctcagccgacaagtccatcagcaccgcctacctgcagtggaccagcctgaaggcctcggacaccgccatgtattactgtgcgagctcacgatattttgactggttactaaatgatgcttttgatatctggggccaagggacaatggtcaccgtctcttca

SEQ ID NO: 105 - DNA, 324 bp, Homo sapiens
gaaattgtgttgacgcagtctccaggcaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccgtcagcagtctggagcctgaagattttgcagtgtattactgtcagcagtatggtagcgcgtcgatcaccttcggccaagggacacgactggagattaaa

SEQ ID NO: 106 - DNA, 360 bp, Homo sapiens
gaggtgcagctggtgcagtctggagcagaggtgaaaaagccgggggagtctctgaagatctcctgtaaggcttctggatacagctttaccaactactggatcggctgggtgcgccagatgcccgggaaaggcctggagtggatggggtacatctatcctggtgactctgataccagatacagcccgtccttccaaggccaggtcaccatctcagccgacaagtccatcaccaccgcctacctgcaatggagcagcctgaaggcctcggacaccgccatgtattattgtgcgagaaaagggtataacagtggcagggagtttgactactggggccagggagccctggtcaccgtctcctca

SEQ ID NO: 107 - DNA, 324 bp, Homo sapiens
gaaattgtgttgacacagtctccagccaccctgtctttgtctccaggggaaagagccaccctctcctgcagggccagtcagagtgttagcaggtacttagcctggtaccagcagaaacctggccaggctcccaggctcctcatctatgatgcatccaacagggccactggcatcccagccaggttcagtggcagtgggcctgggacagacttcactctcaccatcagcagcctagagcctgaagattttgcagtttattactgtcagcagcgtagcaactggcatccattcactttcggccctggggccaaagtggatatcaaa

SEQ ID NO: 108 - DNA, 334 bp, Homo sapiens
cagcctgtgctgactcaatcatcctctgcctctgcttccctgggatcctcggtcaagctcacctgcactctgagcagtgggcacagtagctacatcatcgcatggcatcagcagcagccagggaaggcccctcggtacttgatgaagcttgaaggtagtggaagctacaacaaggggagcggggttcctgatcgcttctcaggctccagctctggggctgaccgctacctcaccatctccaacctccagtctgaggatgaggctgattattactgtgagacctgggacagtaacctttgggtgttcggcggagggaccaagctgaccgtcctag

SEQ ID NO: 109 - DNA, 372 bp, Homo sapiens
caggtgcagctgcaggagtcgggcccaggactggtgaagccttcacagaccctgtccctcacctgcactgtctctggtggctccatcagcagtggtagttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcagacacggccgtgtattactgtgcgagagatcctgggtatagcagcagctggtacggacggtttgactactggggccagggaactctggtcaccgtctcctca

SEQ ID NO: 110 - DNA, 366 bp, Homo sapiens
gaggtgcagctgttggagtctgggggaggcttggtacagcctggggggtccctgagactctcctgtgtagcctctggattcacctttagcagatatgccatgaactgggtccgccaggctccagagagggggctggagtgggtctcaggtattggcactagtgatagttacacatactacgcagactccgtgaagggccggttcaccatctccagagacaattccaagaacacggtgtctctgcaaatcaacagcctgagagccgacgacacggccgtatattactgtgcgaaagatagagatccttcaacttatggttactactttgactactggggccagggaaccctggtcaccgtctcctca

SEQ ID NO: 111 - DNA, 337 bp, Homo sapiens
cagcttgtgctgactcaatcgccctctgcctctgcctccctgggagcctcggtcaagctcacctgcactctgagcagtggacacagcaccaacgccatcgcatggcatcagcagcagccagagaagggccctcgatacttgatgaaaattaatagtgatggcagccacagcaagggggacgggatccctgatcgcttctcaggctccagctctggggctgagcgctacctcaccatctccagcctccagtctgaggatgaggctgactattactgtcagacatggggcgctggcattcaaggagtgttcggcggagggaccaagctgaccgtcctag

SEQ ID NO: 112 - DNA, 330 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcccatgtacacttttggccaggggaccaaggtggagatcaaa

SEQ ID NO: 113 - DNA, 360 bp, Homo sapiens
gaggtgcagctgttggagtctgggggaggcttggtacagcctggggggtccctgagactctcctgtgcagcctctggattcacctttagcagctttgccatgagctgggtccgccaggctccagggaaagggctggagtgggtctcagctattattaatactggtggtagcacatactacgcagactccgtgaagggccggttcaccacctccagagacaattccaagaacatgctgtatctgcaaatgaacagcctgagagccgaggacacggccgtatattactgtgtgaaatggttccactatgatactagtgggggccctggctggggccagggaaccctggtcaccgtctcctca

SEQ ID NO: 114 - DNA, 321 bp, Homo sapiens
gacatccagatgacccagtctccttccaccctgtctgcatctgtaggagacagagtcaccataacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatacttacccgtggacgttcggccaagggaccaaggtggagatcaaa

SEQ ID NO: 115 - DNA, 369 bp, Homo sapiens
caggtgcagctgcaggagtcgggcccaggactggtgaagccttcagagaccctgtccctcacctgcagtgtctctggtggctccatcagtagtggtggtaactactggagctggatccggcagcccgccgggaagggactggagtggattgggcgtatatatgcaagtgggagcaccaactacaacccctccctcaagagtcgagtcaccatgtccttagacacgtccaagaaccagttctccctgaagctgagctctgtgaccgccgcagacacggccgtgtattactgtgcgagattttactatgatggtagtagtttttatccggaggatccctggggccagggaaccctggtcaccgtctcctca

SEQ ID NO: 116 - DNA, 339 bp, Homo sapiens
gacatcgtgatgacccagtctccagactccctggctgtgtctctgggcgagagggccaccatcaactgcaagtccagccagagtgttttatacagctccaacaataagaattccttagcttggtaccagcagaaaccaggacagcctcctaagttgctcatttactgggcatctacccgggaatccggggtccctgaccgattcagtggcagcgggtctgggacagatttcactctcaccatcagcagcctgcaggctgaagacgtggcagtttattactgtcagcaatattgtagtgctcctcgaacgttcggccaagggaccaaggtggaaatcaaa

SEQ ID NO: 117 - DNA, 359 bp, Homo sapiens
aggtgcagctggtgcagtctggggctgaggtgaagaagcctggggcctcagtgaaggtctcctgcaaggcttctggatacaccttccgcagttatgatatcaactgggtgcgacaggccactggacaagggcttgagtggatgggatggatgaaccctgacagtggtgacacaggctatgtaccgaagttccagggcagagtcaccatgaccagggacacctccacaagcacagcctacatggagttgagtagcctgagatctgaggacacggccgtgtattactgtgcgagaggcccctttggtatggggagttattttgactactggggccagggaagtctggtcaccgtctcctca

SEQ ID NO: 118 - DNA, 318 bp, Homo sapiens
gacatccagatgacccagtctccatcctccctgtctgcatctgtaggagacagagtcaccatcacttgccgggcaagtcagagcattagcagctatttaaattggtatcagcagagaccagggaaagcccctaagctcctgatctatgctgcatccactttgcaaaatggggtcccatcaagattcagtggcagtggatctgggacagatttcactctcaccatcagcagtctgcaacctgaagattttgcaacttattcctgtcaacagagttacagtacccgcagttttggccaggggaccaaggtggagatcaaa

SEQ ID NO: 119 - DNA, 379 bp, Homo sapiens
gtgcagctggtggagtcggggggaggcgtggtccagcctgggaggtccctgagactctcctgtgcagcctctggattcagcttcagtaactatttcatgcactgggtccgccatgttccaggcaaggggctggagtggctggcaaccatctggtatgatggaagtgataaatactatgtagactccgtgaagggccgattcaccatctccagagacaactccaagaacacgctctatctacaaatgggcagcctgagagctgaggacacggctatgtattactgtgcgaaagggagacaacgaggatattactacgatatgggtggttattatgtttttgactcctggggccagggaaccctggtcaccgtctcctcag

SEQ ID NO: 120 - DNA, 331 bp, Homo sapiens
cagtctgtgctgacgcagccaccctcagcgtctgggacccccgggcagggcgtcaccatctcttgttctggaagcaggtccaacatcggaagtaatactgttaactggtaccggcagttcccaggaacggcccccaaactcctcatctataagaacaatcagcggccctcaggggtccctgaccgattctctggctccaagcctggcacctcagcctacctggccatcactgggctccagtctgacgatcaggctgattattactgtgcagcatgggatgacagcctgaaaggtgtggtgttcggcggagggaccaagctgaccgtcctaa

SEQ ID NO: 121 - DNA, 355 bp, Homo sapiens
gaggtgcagctggtgcagtctggggctgaggtgaagaagactgggtcctcagtgaggctttcctgcaagacctccggatacaccttcacataccgctacctgcactgggtgcgacaggcccccggacacgcgcttgagtggctggggtggattacgatttataatggcaacaccaagtatgcacaggaattccaggacagagtcaccattaccagggaaatgtccttgaccgcagtatacatggagttgaggagcctaagatctgaggacacagccatgtattactgtgcaaggtccgcgttgtctggcgataatgcctttgaattctgggcccaagggacaatggtcaccgtct

SEQ ID NO: 122 - DNA, 331 bp, Homo sapiens
cagactgtggtgactcaggagccctcactgactgtgtccccaggagagacagtcactctcacctgtggctccaacagtggagctgtcaccagtggtcatcatccccactggttccagcagaagcctggccaagcacccaggactctcatttatgacacaaccaacaaggtctcctggacacctgcccgcttctcagcctccctccttgggggcaaagctgccctgaccctttcgggtgcgcagcctgaggatgaggctgactactattgcttggtctcctatagtggtggtcgtatggtcttcggcggagggaccaagctgaccgtcctac

SEQ ID NO: 123 - DNA, 367 bp, Homo sapiens
gaggtgcagctggtgcagtctggggctgaggtgaagaagactgggtcctcagtgaagatttcctgcaaggcttccggatacaccttcacctacccctacctccactgggtacgccaggcccccggacaagcccttgagtggatgggatggatcacaccctacaatggtaacaccaactacgcacagaaattccaggacagactcaccatcaccagggacacgtctataaacccagtctacatggagctgagcagcctgagatctgaggacacagccatgtattattgtgcaagatctgctttagattgtagtagtgtcagatgcttttcctttgactcctggggccagggaaccctggtcaccgtct

SEQ ID NO: 124 - DNA, 331 bp, Homo sapiens
cagtctgccctgactcagcctcgctcagtgtccgggtctcctggacagtcagtcaccatctcctgcactggaaccagcagtgatgttggtggttataactatgtctcctggtaccaacaacacccaggcaaagtccccaaactcatgatttatgatgtcaatcagcggccctcaggggtccctgctcgcttctctggctccaagtctggcaacacggcctccctgaccatctctggcctccaggctgaagatgaggctgattatcactgttgctcatatgcaggcgcctatacttttgtattcggcggagggaccaagctgaccgtcctag

SEQ ID NO: 125 - DNA, 355 bp, Homo sapiens
gaggtgcagctggtgcagtctggggctgaggtgaggaagactgggtcctcagtgaaactttcctgcgcgacttccggatacactttcacctaccgctacctgcactgggtgcgacaggcccccggccaagcgcttgagtggatgggctggatcacaatttacaatggtaacaccaattacgcacagaaatttcaagacagagtcaccatttccagggacatgtctctgagagcggtctacatggagctgcgcagcctgagatcagacgacacggccatgtatttttgtgtaagatccgcactgtatggggaaaatgcttttgatttctggggccaagggacaatggtcaccgtct

SEQ ID NO: 126 - DNA, 331 bp, Homo sapiens
caggctgtggtgacccaggagccctcactgactgtgtccccaggagggacagtcactctcacctgtgactccgacactggacctgtcaccagtgctcattctccccactggttccaacagaagcctggccaagcccccaggacactgatttatgatacatccaagaagtcttcctggacacctgcccggttctcaggctccctccttgggggcaaagctgccctgaccctctcgggtgcgcagcctgaggatgaggctgactattactgcatcctctccaatagtggagtccattttctcttcggcggggggacccggctgaccgtcctag

SEQ ID NO: 127 - DNA, 352 bp, Homo sapiens
gaggtgcagctggtggagtctgggggaggcgtggcccagcctgggacgtctctgagactctcctgtgaagcgtctggattcaccttccgtgactatgccatgcgctgggtccgccaggctccaggcaaggggctggagtgggtggcatttatatggaatgatggaagtaagaaatattacacagactccgtgaggggccgattcaccatctccagagacaattccaagaacatgctgtatctgcaaatggacagcctgagagccgaggacacggctctttattactgtgccagaaagatgagtgaagatgatgcttttgatctgtggggccaagggacaatggtcaccgtct

SEQ ID NO: 128 - DNA, 324 bp, Homo sapiens
gacatccagatgacccagtctccttccaccctgtctgcatctgcaggagacagagtcaccatcacttgccgggccagtcagagtgttagtaagtacttggcctggtatcagcagaaaccagggaaagcccctaggctcctgatctataaggcatctagtttacaaagtggggtcccatcaaggttcagtggcagtggatctgggacagaattcactctcaccatcagcagcctgcagcctgatgattttgctacttattactgccaacactataatacttatccttcgtggacgttcggccaagggaccaaggtggaaatcaaa

SEQ ID NO: 129 - DNA, 367 bp, Homo sapiens
gaggtgcagctggtgcagtctggggctgaggtgaagaagactgggtcctcagtgaaggtttcttgcaaggcttccggatacaccttcacctacccctacctgcactgggtgcgacaggcccccggacaagcgcttgagtggatgggatggatcacaccctacaatggtaacaccaactacgcacagaaattccaggacagagtcaccataaccagggacacgtctatgaacccagtctacatggagttgatcaacttgcgatctgaagacacagccatgtattactgtgcaagatctgctttagactgtagtagtgccagttgcttatcctttgactcctggggccagggaaccctggtcaccgtct

SEQ ID NO: 130 - DNA, 331 bp, Homo sapiens
cagtctgccctgactcagcctcgctcagtgtccgggtctcctggacagtcagtcaccatctcctgcactggaaccagcagtgatgttggtgcttacaactatgtctcctggtaccaacaacacccaggcaaagcccccaaactcatgatttatgatgtcactcagcggccctcaggggtccctgatcgcttctctggctccaagtctggcaacagggcctccctgaccatctctgggctccagggtgaagatgaggctgattattactgctgctcatatgcagccacctccagtcttttattcggcggagggaccaagctgatcgtcctgc

SEQ ID NO: 131 - DNA, 370 bp, Homo sapiens
gaggtgcagctggtggagtctgggggaggcctggtcaagcctggggggtcgctgagactctcctgtgaagcctctggattcgtcttcaggaattatggcatgaactgggtccgccaggctccagggaaggggctggagtgggtctcatccattagtagtggtagtgcttacacatattacgcagactcagtgaagggccggttcaccatctccagagacaacgccgacaactcactgtatcttcaaatgaacagcctgagagccgaggacacggctgtgtatttttgtgcgaaagatcctgtcgactatcgctacgtcgacgaatactggtacttcgatgtctggggccgtggcaccctggtcaccgtct

SEQ ID NO: 132 - DNA, 321 bp, Homo sapiens
gaaattgtgttgacgcagtctccaggcaccctgtctttgtctccaggggaaagagccaccctctcctgcagggccagtcagagtgttagggacgactacttagcctggtatcagcagaaacctggccaggctcccaggctcctcatctatgctgcgtcccgcagggccactggcgtcccagacaggttctggggcagtgggtcttggacagacttcactctcaccgtcaacagactggagcctgaagattttgcagtgtattactgtcagcagtatggtaactcactcactttcggcggagggaccaaggtggaaatcaaa

SEQ ID NO: 133 - DNA, 379 bp, Homo sapiens
gaggtgcagctggtggagtctgggggaggcttggtaaagcctggggggtcccttcgactctcctgtgcaggctctggattcactttcagtcaggcctggatgagctgggtccgccagtctgcagggaaggggctggagtgggttggccgtattaaaagcaaaaaacatggtgggacaacagactacgctgcacccgtgaaaggcagattcaccatctcaagagatgattcaaaaaacacgttgtatctgcaaatgagcagcctgaagaccgaggacacagccgtgtattactgtgccatagatgattattataatcttgataaagcaggcccctttgatgcttttgatttctggggccaagggacaatggtcaccgtct

SEQ ID NO: 134 - DNA, 318 bp, Homo sapiens
gaaatagtgatgacgcagtctccagccaccctgtctctgtctccaggggaaagagccaccctctcctgcagggccagtcagagtgttgacagcaacttggcctggtaccagcagaaacctggccaggctcccaggctcctcatttatgctgcatccaccagggccactggtatcccagccaggttccgtggcagtgggtctgggacggagttcactctcaccatcagcagcgtgcagtctgaagattctgcggtttattactgtcagcagtataataactggcctttcttcggcggtgggaccaaggtggaaatcaaa

SEQ ID NO: 135 - DNA, 343 bp, Homo sapiens
gaggtgcagctggtggagtctgggggaggcttggtccagcctggggggtccctgagactctcctgtgcagcctctggattcacctttactaattattggctgagttgggtccgccaggctccagggaaggggctggagtgggtggccaacataaacgtaaatggaagtgagaaaaattatgtggactctgtgaggggccgattcaccatctccagagacaacgccaagaactcagtgtatctgcaaatgaacagcctgagagtcgaggacacggccgtttattactgtgcgagagctcttgactacgcgcgctgttggggccagggaaccctggtcaccgtct

SEQ ID NO: 136 - DNA, 321 bp, Homo sapiens
gacatccagatgacccagtctccttccaccctgtctgcatctgtaggagacagagtcaccatcacttgccgggccagtcagaatattagtagttggttggcctggtatcagcagagaccagggaaagcccttaaactcctgatctataaggcgtccagtttagaaagtggggtcccatcaaggttcagcggcagtggatctgggacagaattcactcttaccatcagcagcctgcagcctgatgattttgcaacttatcactgccaacaatataacagttatccctggacgttcggccaagggaccaaggtggaaatcaaa

SEQ ID NO: 137 - DNA, 349 bp, Homo sapiens
gaggtgcagctgttggagtctgggggaggcctggtacagccggcggggtccctgagactcgcctgtgcagcctctggattcacctttagcaactatgccatgacttggatccgccaggctccagggaaggggctggagtgggtctcaactattagtagtggtggttataatacaaactacgcagactccgtgaagggccggttcaccatttctagagacaattccaagaacacggtgtatctgcaaatgagcagcctgagagtcgaggacacggccgtatattactgtgtgactcggagagctgggagctactatgcctactggggccagggaaccctggtcaccgtct

SEQ ID NO: 138 - DNA, 321 bp, Homo sapiens
gacatccagatgacccagtctccttccaccctgtctgcatctgtaggagagagagtcaccatcacttgccgggccagtcagagtattagtagctggttggcctggtatcagcagaaaccagggaaagcccctaacctcctgatctataaggcgtctagtttagaaagtggggtcccatcaaggttcagcggcagtggatctgggacagaattcactctcaccatcagcggcctgcagcctgatgattttgcaacttattactgccaacagtataatagttattcattgacgttcggccaagggaccaaggtggaaatcaaa

SEQ ID NO: 139 - DNA, 358 bp, Homo sapiens
gaggtgcagctggtgcagtccggagcagaggtgaaaaagcccggggagtctctgaggatctcctgtaaggtttctggatacagctttgccagatactggatcagttgggtgcgccaggtgcccgggaaaggcctggagtggatgggaaggattgatcccactgactcttataccacttacagcccgtccttcgagggccacgtcaccatctcagttgacaagtccatcagcactgcctacctgcagtggagcagcctgagggcctcggacaccgccatttattactgtgcgcgagactatttgcaggtggcagcgcctacttttgactactggggccagggaaccctggtcaccgtct

SEQ ID NO: 140 - DNA, 331 bp, Homo sapiens
cagtctgtgctgactcagacaccctcagcgtctgggacccccgggcagagggtcaccatctcttattctggaagcagcgccaatatcggaagtaatactgtaaactggtaccaacacctcccaggaacgacccccaaactcctcatctatagtaataatctgcggccctcaggggtccctgaccgattctctggctccaagtctggcacctcagcctccctggccatcagtgggctccagtctgaggatgaggctgattattactgtgcagcatgggatgacagcctgaaaggttatgtcttcggacctgggaccaaggtcaccgtcctag

SEQ ID NO: 141 - DNA, 355 bp, Homo sapiens
gaggtgcagctggtgcagtccggagcagtggtgaaaaagtccggggagtctctgacgatctcctgtaagacttctggatacgactttcccagctattggatcaactgggtgcgcctggtgcccgggaaaggcctggagtggatggggacgattgatcctagtgaatcctacatcaactacagcccgtccttccaaggacgcgtctccatctctgccgacaagtccatcagaactgttttcctgcagtggagcaacctgagggcctcggacaccgccatctattactgtgcgcgacgttcgagtagagagcaatggccggtaagagactggggccagggaaccctggtcaccgtct

SEQ ID NO: 142 - DNA, 321 bp, Homo sapiens
gacatccagatgacccagtctccttccaccctgtctgcatctgtaggagacagagtcaccatcacttgccgggccagtcagagtattagtaggtggttggcctggtatcagcagaaaccagggaaagcccctaagctcctggtctatgcggcatctagtttacaaagtggggtcccatcaaggttcagcggcactggatctgggtcagaattcagtctcaccatcaacagcctgcagcctgatgattttgcaacttattactgccaatactatagtacttattcgtggacgttcggccaagggaccaaggtggaaatcaaa

SEQ ID NO: 143 - DNA, 358 bp, Homo sapiens
gaggtgcagctgttggagtctgggggaggcttggtacagcctggggggtccctgagactctcctgtgcagcctctggattcagctttagcacttatgccatgagctgggtccgccaggctccaggaaaggggctgcagtgggtctcaagcattagtgggaatggttacagctcatattatgcagactccgtgaagggccgcttccacatctccagagacaattccaagaacacgctgtctctggaagtgaacagtctgggcgccgaggacacggccgtctatttctgtgcgaaaggtgacggtggcagtggctggtttggttttgactattggggccagggaaccctggtcaccgtct

SEQ ID NO: 144 - DNA, 346 bp, Homo sapiens
cagcttgtgctgactcagccgtcttccctgtctgcatctcctggagcatcagccagtctcacctgcaccttacgcagtggcatcaatgttggtatttatagcatttattggtaccagcagaagccagggagtcctccccagtatctcctgaggttcaaatcagactcagataaacgtcagggctctggagtccccagccgcttctctggatccaaagatggttcggccaatgcagcgattttagtcatctctggggtccagtctgaggatgaggctgactactactgtatgatgtggcacagcagcgcttgggtgttcggcggagggaccaagctgaccgtcctac

SEQ ID NO: 145 - DNA, 352 bp, Homo sapiens
gaggtgcagctgttggagtctgggggagacttggcacagcctggggggtccctgagactctcctgtgcagcctccggattcacctttagcgcctatggcatgacctgggtccgccaggctccagggaaggggctggaatgggtctcaacaattgctgataatggtggtgccacacactacgcagagtccgtgaaaggccggttcaccatctccagagacaattccaagaacacattctatctgcaaatgaacagcctgagagacgaggacacggccatatattactgtgcgaaactgtggtggggttccagatgctttgactactggggccagggaaccctggtcaccgtct

SEQ ID NO: 146 - DNA, 336 bp, Homo sapiens
gatattgtgatgacccagactccactctcctcacctgtcacccttggacagccggcctccatctcctgcaggtctagtcaaagcctcgaacacactgatggaaacacctacttgagttggcttcaacagaggccaggccagcctccaagactcctaatttataagatttctaaccggttctctggggtcccagacagattcagtggtagtggggcagggacagatttcacactgaacatcagcagggtggaaggtgaggatgtcggggtttattactgcatgcaaggtacacaatttcctcacacttttggccaggggaccaaggtggagatcaaa

SEQ ID NO: 147 - DNA, 358 bp, Homo sapiens
gaggtgcagctggtggagtctgggggaggcttggtccagcctggagggtccctgagactctcctgtgcagcctctggattcaccttcagtgaccactatatggactgggtccgccaggttccaaggaaggggctggagtgggttggccgtgctagaaacaaagctaacagttacaccacagaatacgccgcgtctgtgaaaggcagattcaccatctcaagagatgattcaaagaactcactgtatctacaaatgaacagcctgaaaagcgaggacacggccgtgtattattgtgctagatttctgcttattgcggatgcttttgatatctggggccaagggacaatggtcaccgtct

SEQ ID NO: 148 - DNA, 321 bp, Homo sapiens
gacatccagatgacccagtctccatcctccctgtctgcatctttaggagacagagtcaccatcacttgccgggcaagtcagggcattagacatgatttaggctggtatcagcagaaaccagggaaagcccctaagctcctcatctatggtgcatccactttacaaactggggtcccatcaaggttcagcggcagtgggtctggcacagatttcactctcaccatcagcagcctgcagcctgaagattttgcaacttattactgtctacaaaagtacaactacccgctcactttcggcggagggaccaaggtggagatcaaa

SEQ ID NO: 149 - DNA, 358 bp, Homo sapiens
gaggtgcagctggtggagtctgggggaggcttggtccagcctggagggtccctgagactctcctgtgcagcctctggattcaccttcagtgactactacatggactgggtccgccaggctccagggaaggggctggagtgggttggccgcactagaaacaaagctaacagttacaccacagaatacgccgcgtctgtgaaaggcagattcaccatctcaagagatgattcaaagaactcactgtatctgcaaatgaacagcctgaaaaccgaggacacggccgtgtattactgtgctagatttctgctggttgcggatgcttttgatatctggggccaagggacaatggtcaccgtct

SEQ ID NO: 150 - DNA, 321 bp, Homo sapiens
gacatccagatgacccagtctccatcctccctgtctgcatccgtaggagacagagtcaccatcacttgccgggcaagtcagggcattagaaatgatttaggctggtatcagcagaaaccagggaaagcccctaagctcctgatctatggtgcatccagtttacaaagtggggtcccatcaagattcagcggcagtggatctggcacagatttcactctcaccatcagcagcctgcagcctgaagatattgcaacctattactgtctacaagaatacacttacccgctcactttcggcggagggaccaaggtggaaatcaaa

SEQ ID NO: 151 - DNA, 346 bp, Homo sapiens
gaggtgcagctggtggagactggaggaggcttgatccagcctggggggtccctgagactctcctgtgcagcctctgggttcaccgtcagtagcaactacatgagctgggtccgccaggctccagggaaggggctggagtgggtctcacttatttatagcggtggtagcacatactacgcagactccgtgaagggccgattcaccatctccagagacaattccaagaacacgctgtatcttcaaatgaacagcctgagagccgaggacacggccgtgtattactgtgcgagagggcgcctaggaggcctttatgactactggggccagggaaccctggtcaccgtct

SEQ ID NO: 152 - DNA, 331 bp, Homo sapiens
cagtctgtgctgacgcagccgccctcagtgtctgcggccccaggacagaaggtcaccatctcctgctctggaagcagctccaacattgggaataattatgtatcctggtaccagcagctcccaggaacagcccccaaactcctcatctatgaaaataataagcgaccctcagggattcctgaccgattctctggctccaagtctggcacgtcagccaccctgggcatcaccggactccagactggggacgaggccgattattactgcggaacatgggatagcagcctgagtgtcgtggtatccggcggagggaccaagctgaccgtcctag

SEQ ID NO: 153 - DNA, 364 bp, Homo sapiens
gaagtgcagctggtggagtctggggggggcgtggtccagcctggggggtccctgagactctcctgtgcagcgtccggattcatcttcagtgactgggccatgcactgggtccgccaggctccaggcaaggggctggagtggctggcatttattcgggatgatggaaacgtgcagcactatgcagactccgtggagggccgattctccgtgtccagagacaactccttaaacacgctgcatctgcacatgaagagtctgacagtggacgacacggctgtctattactgtgcgaaagagacggctctctccacttcgggtagttattactttgactactggggccagggaaccctggtcaccgtct

SEQ ID NO: 154 - DNA, 321 bp, Homo sapiens
gaaattgtgttgacacagtctccagccaccctgtctttgtctccaggggagggagccaccctctcctgcagggccagtcagagtgttgacagctccttagcctggtaccaacagagagctggccagagtcccagcctcctgatctatgattcatccaagagggccactggcatcccagccaggttcagtggcagtgggtctgggacagacttcacgctcaccatcagcagcgtagagcctgaggatgttgcagtttattattgtcaccagcgttacaactggcctaacacttttggccaggggaccaaggtggagatcaaa

SEQ ID NO: 155 - DNA, 355 bp, Homo sapiens
caggtgcagctgcaggagtcgggcccaggactggtgaagccttcggagaccctgtccctcacctgcactgtctctggtggctccatcaatagttactactggagctggatccggcagcccccagggaagggactggagtggattgggtatatctattacagtgggagcaccaactataacccctccctcaagagtcgagtcaccatatcagtagacacgtccaagaaccagttctccctgaggctgagctctgtgaccgccgcagacacggccgtgtattactgtgcgagacaccccaccgcggggacgagctacggtatggacgtctggggccaagggaccacggtcaccgtct

SEQ ID NO: 156 - DNA, 331 bp, Homo sapiens
cagtctgccctgactcagcctgcctccgagtctgggtctcctggacagtcgatcaccatctcctgcactggaaccaccagtgatgttgggagttataaccttgtctcctggtaccaacagcacccaggcaaagcccccaaactcatgatttatgagggcactaagcggccctcaggggtttctaatcgcttctctggctccaagtctggcaacacggcctccctgacaatctctgggctccagcctgaggacgaggctgattattactgctgctcatatgcaggtactaccacttatgtcttcgggactgggaccaaggtcaccgtcctag

SEQ ID NO: 157 - DNA, 349 bp, Homo sapiens
gaggtgcagctggtgcagtctggggctgaggtgaagaagcctggggcctcagtgaaggtttcctgcaaggcatctggatacaccttcaccaactactatatacactgggtgcgacaggcccctggacaagggcttgagtggatgggaataatcacccctagtggtggtatcacaacctacgcacagaagttccagggcagagtcaccatgaccagggacacgtcgacaagcacagtctacatggagctgagcagcctgagatctgaggacacggccgtgtattactgtgcgagagagtacagcccggggtactttgactactggggccagggaaccctggtcaccgtct

SEQ ID NO: 158 - DNA, 328 bp, Homo sapiens
caggctgtggtgactcaggagccctcactgactgtgtccccaggagggacagtcactctcacctgtgcttccagcactggagaagtcaccagtggttactatccaaactggttccagcagaaacctggacaagcacccagggcactgatttataatacaaacaacaaacacccctggacccctgcccggttctcaggctccctccttgggggcaaagctgccctgacactgtcaggtgtgcagcctgaggacgaggctgagtattactgcctgcttcactatgttactgcttgggtgttcggcggagggaccaagctgaccgtcctag

SEQ ID NO: 159 - DNA, 352 bp, Homo sapiens
gaggtgcagctggtgcagtctggggctgaggtgaaacggcctggggcctcaatgaagctttcttgcaagacatttggatacatcttcaccacctactacatacactgggtgcggcaggcccctggacaaggtcttgaatacttgggaataatcaaccctgctgttggtaccacaaacttcgcacagaagttcaagggcaggctcagcatgaccagggacacgtccacgagcacagtctacatggaactgagcagcccgacatctgacgactcggccgtctattactgtgcgagggaactacacgctacacacttcttcgacttctggggccagggaaccctggtcaccgtct

SEQ ID NO: 160 - DNA, 331 bp, Homo sapiens
cagtctgtgctgacgcagccaccctcagcgtctgggacccccgggcagagggtcattatttcttgttctggggggaactccaacatcggaagttatactgtccagtggtaccatcaagtcccaggaacggcccccaaactcctcatctataggagtaatcagcggccctcaggggtccctgaccgattctctggctccaagtctggcacctcagcctccctggccatcagtgggctccagtctgaagatgaggctgattattactgttcagcatgggatgacagcctgaatgcttatgtcttcggaactgggaccagggtcgccgtcctag

SEQ ID NO: 161 - DNA, 355 bp, Homo sapiens
caggtgcagctgcaggagtcgggcccaggactggtgaagccttcggagaccctgtccctcacctgcactgtctctggtggctccatcagtaattactactggagctggattcggcagcccccagggaagggactggagtggcttgggtatgtctattacagtgggagcaccaactacaacccctccctcaagagtcgagtcaccatattagtagacacgtccaagaatcagttctccctgaagttgagctctgtgaccgccgcagacacggccgtttattactgtgcgagacaccccaccgcggggacgagttacggtctgggcgtctggggccaagggaccacggtcaccgtct

SEQ ID NO: 162 - DNA, 331 bp, Homo sapiens
cagtctgccctgactcagcctgcctccgagtctgggtctcctggacagtcgatcaccatctcctgcactggaaccagcagtgatgttgggaattatgaccttgtctcctggtaccaacagcacccaggcaaagcccccaaactcatgatttatgagggcactaagcggccctcaggaatttctaatcgcttctctggctccaagtctggcaacacggcctccctgacaatctctgggctccaggctgaggacgaggctgattattactgctgctcatatgcaggtactaccacttatgtcttcggaactgggaccaaggtcaccgtcctag

SEQ ID NO: 163 - DNA, 349 bp, Homo sapiens
gaggtgcagctggtgcagtctggggctgaggtgaagaagcctggggcctcagtgaaggtttcctgcaaggcatctggatacaccttcaccacacactatatgcactgggtgcgacaggcccctggacaagggcttgagtggatgggaataatcaaccctagtggtggtagcacaaggtacgcacagaagctgcagggcagagtcaccatgaccggggacacgtccacgagcacagtctacatggagctgagcagcctgagatctgaggacacggccgtgtattactgtgcgagagagatggcaggtggttactttgactactggggccagggaaccctggtcaccgtct

SEQ ID NO: 164 - DNA, 328 bp, Homo sapiens
caggctgtggtgacccaggagccctcactgactgtgtccccaggagggacagtcactctcacctgtgcttccagcgctggaacagtcaccagtacttactatccaaactggttccagcagaaacctggacaagcacccagggcactgatttatagtacaagcaacaaacactcctggacccctgcccgcttctcaggctccctccttgggggcaaagctgccctgacactgtcaggtgtgctgcctgaggacgaggctgagtattactgcctgctctactatggttattcttgggtgttcggcggagggaccaagctgaccgtcctag

SEQ ID NO: 165 - protein, 110 aa, Homo sapiens
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTLLFGGGTKLTVL

SEQ ID NO: 166 - protein, 121 aa, Homo sapiens
EVQLVESGGGLVQPGGSLRLSCAASGFTFSSYWMYWVRQAPGKGLVWVSRINSDGSTTNYADSVKGRFTISRDNAKNTLYLQMNSLRAEDTAVYFCARYAHLGIGWYFDLWGRGTLVTVSS

SEQ ID NO: 167 - protein, 108 aa, Homo sapiens
DIQMTQSPSTLSASAGDRVTITCRASQSVSKYLAWYQQKPGKAPRLLIYKASSLQSGVPSRFSGSGSGTEFTLTISSLQPDDFATYYCQHYNTYPSWTFGQGTKVEIK

SEQ ID NO: 168 - protein, 117 aa, Homo sapiens
EVQLVESGGGVAQPGTSLRLSCEASGFTFRDYAMRWVRQAPGKGLEWVAFIWNDGSKKYYTDSVRGRFTISRDNSKNMLYLQMDSLRAEDTALYYCARKMSEDDAFDLWGQGTMVTV

SEQ ID NO: 169 - protein, 107 aa, Homo sapiens
DIQMTQSPSSLSASVGDRVTITCRASQGIRNDLGWYQQKPGKAPKLLIYGASSLQSGVPSRFSGSGSGTDFTLTISSLQPEDIATYYCLQEYTYPLTFGGGTKVEIK

SEQ ID NO: 170 - protein, 119 aa, Homo sapiens
EVQLVESGGGLVQPGGSLRLSCAASGFTFSDYYMDWVRQAPGKGLEWVGRTRNKANSYTTEYAASVKGRFTISRDDSKNSLYLQMNSLKTEDTAVYYCARFLLVADAFDIWGQGTMVTV

SEQ ID NO: 171 - protein, 110 aa, Homo sapiens
QTVVTQEPSLTVSPGETVTLTCGSNSGAVTSGHHPHWFQQKPGQAPRTLIYDTTNKVSWTPARFSASLLGGKAALTLSGAQPEDEADYYCLVSYSGGRMVFGGGTKLTVL

SEQ ID NO: 172 - protein, 118 aa, Homo sapiens
EVQLVQSGAEVKKTGSSVRLSCKTSGYTFTYRYLHWVRQAPGHALEWLGWITIYNGNTKYAQEFQDRVTITREMSLTAVYMELRSLRSEDTAMYYCARSALSGDNAFEFWAQGTMVTV

SEQ ID NO: 173 - protein, 107 aa, Homo sapiens
DIQMTQSPSSLSASLGDRVTITCRASQGIRHDLGWYQQKPGKAPKLLIYGASTLQTGVPSRFSGSGSGTDFTLTISSLQPEDFATYYCLQKYNYPLTFGGGTKVEIK

SEQ ID NO: 174 - protein, 119 aa, Homo sapiens
EVQLVESGGGLVQPGGSLRLSCAASGFTFSDHYMDWVRQVPRKGLEWVGRARNKANSYTTEYAASVKGRFTISRDDSKNSLYLQMNSLKSEDTAVYYCARFLLIADAFDIWGQGTMVTV

SEQ ID NO: 175 - protein, 110 aa, Homo sapiens
QSVLTQPPSASGTPGQGVTISCSGSRSNIGSNTVNWYRQFPGTAPKLLIYKNNQRPSGVPDRFSGSKPGTSAYLAITGLQSDDQADYYCAAWDDSLKGVVFGGGTKLTVL

SEQ ID NO: 176 - protein, 126 aa, Homo sapiens
VQLVESGGGVVQPGRSLRLSCAASGFSFSNYFMHWVRHVPGKGLEWLATIWYDGSDKYYVDSVKGRFTISRDNSKNTLYLQMGSLRAEDTAMYYCAKGRQRGYYYDMGGYYVFDSWGQGTLVTVSS

SEQ ID NO: 177 - protein, 107 aa, Homo sapiens
DIQMTQSPSSLSASVGDRVTITCRASQSVSRYLNWYQQKPGRAPRLLIFGASSLRSGVPSRFSGSGSGTDFSLTINSLQPEDFATYYCQQTYSTFGPFGPGTKVDIK

SEQ ID NO: 178 - protein, 124 aa, Homo sapiens
EVQLVESGGGLVKPGGSLRLSCAASGFTFSSAWLSWVRQDPGKGLEWLGRIKSKAAGGTADHAASVKGRFTISRDDSKNTLYLQMNSLKIEDTAVYYCTTGSLEGTITHAEDHWGQGTLVTVSS

SEQ ID NO: 179 - protein, 107 aa, Homo sapiens
DIQMTQSPSTLSASVGERVTITCRASQSISSWLAWYQQKPGKAPNLLIYKASSLESGVPSRFSGSGSGTEFTLTISGLQPDDFATYYCQQYNSYSLTFGQGTKVEIK

SEQ ID NO: 180 - protein, 116 aa, Homo sapiens
EVQLLESGGGLVQPAGSLRLACAASGFTFSNYAMTWIRQAPGKGLEWVSTISSGGYNTNYADSVKGRFTISRDNSKNTVYLQMSSLRVEDTAVYYCVTRRAGSYYAYWGQGTLVTV

SEQ ID NO: 181 - protein, 112 aa, Homo sapiens
DIVMTQSPLSLPVTLGQPASISCRSSQSLAYSDGNTYLNWFHQRPGHSPRRLIYKVFNRDSGVPDRFSGSGSGTDFTLKISRVEAEDVGVYYCMQGTHWPITFGQGTRLEIK

SEQ ID NO: 182 - protein, 121 aa, Homo sapiens
QVQLVQSGAEVKKPGAPVKVSCKASGYTFTNYYMHWVRQAPGQGLEWLGLINAGSGRTRYAPKFQGRVTMTRDTSTSTLYIEVDSLRSDDTAVYYCARGSAHIEAGGSAFDKWGQGTLVTV

SEQ ID NO: 183 - protein, 112 aa, Homo sapiens
DIVMTQSPLSLPVILGQSASISCRSSQSLIHSDGNIFLNWFQQRPGQSPRRLIYKVSNRDSGVPDRFSGSGSGTDFTLKISRVEAEDVGVYYCMQGTHWPITFGQGTRLEIK

SEQ ID NO: 184 - protein, 121 aa, Homo sapiens
QVQLVQSGAEVKKPGAPVKVSCQASGFIFINYYFHWVRQAPGQGLEWMGVINPTSGKSKLAPTLENRVTMTSDTSTRTLYIEVTSLTSDDTAIYYCARGSARAEAAGSAFDHWGQGTLVTV

SEQ ID NO: 185 - protein, 107 aa, Homo sapiens
DIQMTQSPSSLSASVGDRVTITCQASLDIKNYLNWYQQKPGEAPKLLISDASTLETGVPSRFSGRGSGTHFTVTIRSLQSEDVATYYCQQYGKLPFTFGPGTKVDIK

SEQ ID NO: 186 - protein, 124 aa, Homo sapiens
QVQLQESGPGLVKPSETLSLTCTVSGASMNGYYWNWIRQPPGKGLEWIGHIHYVGTTMKATNDSPSLRSRVTISVDTSRSQISLKLTSVTAADTAVYYCARRRYHFWSDPGTFDIWGQGTMVTV

SEQ ID NO: 187 - protein, 115 aa, Homo sapiens
QLVLTQPSSLSASPGASASLTCTLRSGINVGIYSIYWYQQKPGSPPQYLLRFKSDSDKRQGSGVPSRFSGSKDGSANAAILVISGVQSEDEADYYCMMWHSSAWVFGGGTKLTVL

SEQ ID NO: 188 - protein, 119 aa, Homo sapiens
EVQLLESGGGLVQPGGSLRLSCAASGFSFSTYAMSWVRQAPGKGLQWVSSISGNGYSSYYADSVKGRFHISRDNSKNTLSLEVNSLGAEDTAVYFCAKGDGGSGWFGFDYWGQGTLVTV

SEQ ID NO: 189 - protein, 110 aa, Homo sapiens
QSVLTQPPSASGTPGQRVIISCSGGNSNIGSYTVQWYHQVPGTAPKLLIYRSNQRPSGVPDRFSGSKSGTSASLAISGLQSEDEADYYCSAWDDSLNAYVFGTGTRVAVL

SEQ ID NO: 190 - protein, 117 aa, Homo sapiens
EVQLVQSGAEVKRPGASMKLSCKTFGYIFTTYYIHWVRQAPGQGLEYLGIINPAVGTTNFAQKFKGRLSMTRDTSTSTVYMELSSPTSDDSAVYYCARELHATHFFDFWGQGTLVTV

SEQ ID NO: 191 - protein, 77 aa, Artificial Sequence
  synthetic
AGKPAVIPDREALYQDIDEMEECLDEAGKPAVIPDREALYQDIDEMEECLDEAGKPAVIPDREALYQDIDEMEECLD

SEQ ID NO: 192 - protein, 5 aa, Artificial Sequence
  synthetic
ALYQI

SEQ ID NO: 193 - protein, 21 aa, Hepacivirus C (human)
KPAVIPDREVLYQAFDEMEEC

SEQ ID NO: 194 - protein, 21 aa, Hepacivirus A (non-primate, canine, equine)
KVVVVPHKGVLYADFDEMEEC

SEQ ID NO: 195 - protein, 21 aa, Hepacivirus K (bat)
KIFISQDRDNLYTILDEMEEC

SEQ ID NO: 196 - protein, 21 aa, Hepacivirus M (bat)
KIFLSPDKDHLYGWFEEMEEC

SEQ ID NO: 197 - protein, 21 aa, Hepacivirus L (bat)
TTLEDADMPAYLTDMGELEEC

SEQ ID NO: 198 - protein, 21 aa, Hepacivirus E (rodent)
TTAARVVQPPEDDITENLEEC

SEQ ID NO: 199 - protein, 21 aa, Hepacivirus F (rodent)
STAARVVEFEPLDTEEVLEEC

SEQ ID NO: 200 - protein, 21 aa, Hepacivirus J (rodent)
FVNTLPPEVLFELEPTEAEEC

SEQ ID NO: 201 - protein, 21 aa, Hepacivirus I (rodent)
CRPELRLNETIDEPAIELEEC

SEQ ID NO: 202 - protein, 21 aa, Hepacivirus G (Norway rat 1)
DSSAALNPAPEMSILEEVEEC

SEQ ID NO: 203 - protein, 21 aa, Hepacivirus H (Norway rat 2)
  misc_feature (7)..(7): Xaa can be any naturally occurring amino acid
AKGAEPXPLEQEMDTAGMEEC

SEQ ID NO: 204 - protein, 21 aa, Hepacivirus N (bovine)
DSGEPPNAPEPADDDDGFEEC

SEQ ID NO: 205 - protein, 21 aa, Hepacivirus D (Guereza, Old World monkey)
QAERAQVMEDFIGDLIETEEC

SEQ ID NO: 206 - protein, 21 aa, Sifaka hepacivirus (lemur)
TTSALHAPPPPISLEGALEEC

SEQ ID NO: 207 - protein, 21 aa, Hepacivirus B (GB virus-B; human)
SVPTGATVAPVVDEEEIVEEC

SEQ ID NO: 208 - protein, 21 aa, Hepacivirus P (ground squirrel)
STASLASQPIDDPLTSGLEEC

SEQ ID NO: 209 - protein, 21 aa, Hepacivirus C
KPAVIPDREVLYQGFDEMEEC

SEQ ID NO: 210 - protein, 21 aa, Hepacivirus C
KPAVIPDREALYQDIDEMEEC

SEQ ID NO: 211 - protein, 21 aa, Artificial Sequence
  synthetic
IYPLEDMAEPKVERIDAQEDC

SEQ ID NO: 212 - protein, 17 aa, Artificial Sequence
  synthetic
AVIPDREALYQDIDEME

SEQ ID NO: 213 - protein, 17 aa, Hepatitis C
KPAVIPDREVLYQGFDE

SEQ ID NO: 214 - protein, 17 aa, Hepatitis C
RPAIIPDREALYQEFDE

SEQ ID NO: 215 - protein, 17 aa, Hepatitis C
KPAVIPDREVLYQAFDE

SEQ ID NO: 216 - protein, 17 aa, Hepatitis C
KPAVVPDREVLYQEFDE

SEQ ID NO: 217 - protein, 17 aa, Hepatitis C
SPAIVPDREVLYQDFDE

SEQ ID NO: 218 - protein, 17 aa, Hepatitis C
EALYQQFDEMEECSASL

SEQ ID NO: 219 - protein, 17 aa, Hepatitis C
AIIPDREALYQEFDEME

SEQ ID NO: 220 - protein, 17 aa, Hepatitis C
IVPDREVLYQDFDEMEE

SEQ ID NO: 221 - protein, 17 aa, Hepatitis C
RPAIVPDRELLYQEFDE

SEQ ID NO: 222 - protein, 17 aa, Hepatitis C
IVPDRELLYQEFDEMEE

SEQ ID NO: 223 - protein, 17 aa, Hepatitis C
PAIVPDREVLYQDFDEM

SEQ ID NO: 224 - protein, 17 aa, Hepatitis C
EVLYQDFDEMEECASHL

SEQ ID NO: 225 - protein, 17 aa, Hepatitis C
AIVPDRELLYQEFDEME

SEQ ID NO: 226 - protein, 17 aa, Hepatitis C
RPAIVPDREVLYQEFDE

SEQ ID NO: 227 - protein, 17 aa, Hepatitis C
IIPDREALYQEFDEMEE

SEQ ID NO: 228 - protein, 17 aa, Hepatitis C
RPAVIPDREVLYQEFDE

SEQ ID NO: 229 - protein, 17 aa, Hepatitis C
PDREALYQEFDEMEECA

SEQ ID NO: 230 - protein, 17 aa, Hepatitis C
RPAIIPDREVLYQEFDE

SEQ ID NO: 231 - protein, 17 aa, Hepatitis C
REALYQEFDEMEECASH

SEQ ID NO: 232 - protein, 17 aa, Hepatitis C
KPALVPDKEVLYQQYDE

SEQ ID NO: 233 - protein, 17 aa, Hepatitis C
PAIIPDREALYQEFDEM

SEQ ID NO: 234 - protein, 17 aa, Hepatitis C
PDREALYQQFDEMEECS

SEQ ID NO: 235 - protein, 21 aa, Artificial Sequence
  synthetic
EDECIPNAVHLTFQGSWMKRY

SEQ ID NO: 236 - protein, 21 aa, Artificial Sequence
  synthetic
MDPCEGIMTSQAHNVWLYKFR

SEQ ID NO: 237 - protein, 21 aa, Artificial Sequence
  synthetic
EDETCIVSPHMYQNLFAWGKR

SEQ ID NO: 238 - protein, 21 aa, Artificial Sequence
  synthetic
DDEVICATHSPYGQFWMNLRK

SEQ ID NO: 239 - protein, 21 aa, Artificial Sequence
  synthetic
FIVFLCYDEHTWASPGQMNRK

SEQ ID NO: 240 - protein, 21 aa, Artificial Sequence
  synthetic
EDTSCGEAFMYNWHQRVKIPL

SEQ ID NO: 241 - protein, 21 aa, Artificial Sequence
  synthetic
QQEIVDFLMTPNCYHWSRAGK

SEQ ID NO: 242 - protein, 21 aa, Artificial Sequence
  synthetic
YIHTEDYVCLSFNQWAMRKPG

SEQ ID NO: 243 - protein, 21 aa, Artificial Sequence
  synthetic
LLFECDMHYWVTSRQPNAGIK

SEQ ID NO: 244 - protein, 21 aa, Artificial Sequence
  synthetic
AEMCAFQDYIHRVTSLNGWKP

SEQ ID NO: 245 - protein, 21 aa, Artificial Sequence
  synthetic
EEPADGCQHMNIKFLYWVTSR

SEQ ID NO: 246 - protein, 21 aa, Artificial Sequence
  synthetic
REDCQMAHPVTRIGSNKFWLY

SEQ ID NO: 247 - protein, 21 aa, Artificial Sequence
  synthetic
DCIEDAWHQPGVSMLTRNFKY

SEQ ID NO: 248 - protein, 21 aa, Artificial Sequence
  synthetic
PCDEWHIVAGTRPFMLSNKQY

SEQ ID NO: 249 - protein, 21 aa, Artificial Sequence
  synthetic
IDWCAEGQPHVNTFIRLMSKY

SEQ ID NO: 250 - protein, 21 aa, Artificial Sequence
  synthetic
ICDVAHESGWINFRQTKLPMY

SEQ ID NO: 251 - protein, 21 aa, Artificial Sequence
  synthetic
ACDIEHFLARWGQTVKPNSMY

SEQ ID NO: 252 - protein, 21 aa, Artificial Sequence
  synthetic
ETVISDEPFAMYLCGHNRQWK

SEQ ID NO: 253 - protein, 21 aa, Artificial Sequence
  synthetic
MTSMIVFDGAEPCYLRHNQWK

SEQ ID NO: 254 - protein, 21 aa, Artificial Sequence
  synthetic
ETSFYIDEVMLPACHWNRQGK

SEQ ID NO: 255 - protein, 21 aa, Artificial Sequence
  synthetic
DVISAETDCLMQFYHWGKNRP

SEQ ID NO: 256 - protein, 21 aa, Artificial Sequence
  synthetic
IIFYVRQLWNHTCKESPADGM

SEQ ID NO: 257 - protein, 21 aa, Artificial Sequence
  synthetic
DSTAFMRDYQKENCGLWHIVP

SEQ ID NO: 258 - protein, 21 aa, Artificial Sequence
  synthetic
QQYWVTSRPNACDEFGHIKLM

SEQ ID NO: 259 - protein, 21 aa, Artificial Sequence
  synthetic
YFYIHWLTVKRNMCESPQADG

SEQ ID NO: 260 - protein, 21 aa, Artificial Sequence
  synthetic
LLYWVTSRQPNACDEFGHIKM

SEQ ID NO: 261 - protein, 21 aa, Artificial Sequence
  synthetic
AMAQFSYETLRNIKHVWCGDP

SEQ ID NO: 262 - protein, 21 aa, Artificial Sequence
  synthetic
EPEGNAQMDHIVFSRCLTKYW

SEQ ID NO: 263 - protein, 21 aa, Artificial Sequence
  synthetic
RGEPQTMRNDHVSAIFKCLYW

SEQ ID NO: 264 - protein, 21 aa, Artificial Sequence
  synthetic
DEDGTVPMQNISAHLRFWYCK

SEQ ID NO: 265 - protein, 21 aa, Artificial Sequence
  synthetic
PGETMPNFSDIHWYRLCQVAK

SEQ ID NO: 266 - protein, 21 aa, Artificial Sequence
  synthetic
ITEGSNYMWHFRPIQDLCVAK

SEQ ID NO: 267 - protein, 21 aa, Artificial Sequence
  synthetic
VEDHMGFTSCPIQNLYRWAVK

SEQ ID NO: 268 - protein, 21 aa, Artificial Sequence
  synthetic
ADECHTISGFMLNWRQPYVKA

SEQ ID NO: 269 - protein, 9 aa, Homo sapiens
SSDVGGYNY

SEQ ID NO: 270 - protein, 3 aa, Homo sapiens
DVS

SEQ ID NO: 271 - protein, 10 aa, Homo sapiens
CSYAGSYTLL

SEQ ID NO: 272 - protein, 8 aa, Homo sapiens
GFTFSSYW

SEQ ID NO: 273 - protein, 8 aa, Homo sapiens
INSDGSTT

SEQ ID NO: 274 - protein, 14 aa, Homo sapiens
ARYAHLGIGWYFDL

SEQ ID NO: 275 - protein, 6 aa, Homo sapiens
QSVSKY

SEQ ID NO: 276 - protein, 3 aa, Homo sapiens
KAS

SEQ ID NO: 277 - protein, 10 aa, Homo sapiens
QHYNTYPSWT

SEQ ID NO: 278 - protein, 8 aa, Homo sapiens
GFTFRDYA

SEQ ID NO: 279 - protein, 8 aa, Homo sapiens
IWNDGSKK

SEQ ID NO: 280 - protein, 12 aa, Homo sapiens
ARKMSEDDAFDL

SEQ ID NO: 281 - protein, 6 aa, Homo sapiens
QGIRND

SEQ ID NO: 282 - protein, 3 aa, Homo sapiens
GAS

SEQ ID NO: 283 - protein, 9 aa, Homo sapiens
LQEYTYPLT

SEQ ID NO: 284 - protein, 8 aa, Homo sapiens
GFTFSDYY

SEQ ID NO: 285 - protein, 10 aa, Homo sapiens
TRNKANSYTT

SEQ ID NO: 286 - protein, 12 aa, Homo sapiens
ARFLLVADAFDI

SEQ ID NO: 287 - protein, 9 aa, Homo sapiens
SGAVTSGHH

SEQ ID NO: 288 - protein, 3 aa, Homo sapiens
DTT

SEQ ID NO: 289 - protein, 10 aa, Homo sapiens
LVSYSGGRMV

SEQ ID NO: 290 - protein, 8 aa, Homo sapiens
GYTFTYRY

SEQ ID NO: 291 - protein, 8 aa, Homo sapiens
ITIYNGNT

SEQ ID NO: 292 - protein, 13 aa, Homo sapiens
ARSALSGDNAFEF

SEQ ID NO: 293 - protein, 6 aa, Homo sapiens
QGIRHD

SEQ ID NO: 294 - protein, 9 aa, Homo sapiens
LQKYNYPLT

SEQ ID NO: 295 - protein, 8 aa, Homo sapiens
GFTFSDHY

SEQ ID NO: 296 - protein, 10 aa, Homo sapiens
ARNKANSYTT

SEQ ID NO: 297 - protein, 12 aa, Homo sapiens
ARFLLIADAFDI

SEQ ID NO: 298 - protein, 8 aa, Homo sapiens
RSNIGSNT

SEQ ID NO: 299 - protein, 3 aa, Homo sapiens
KNN

SEQ ID NO: 300 - protein, 11 aa, Homo sapiens
AAWDDSLKGVV

SEQ ID NO: 301 - protein, 8 aa, Homo sapiens
GFSFSNYF

SEQ ID NO: 302 - protein, 8 aa, Homo sapiens
IWYDGSDK

SEQ ID NO: 303 - protein, 20 aa, Homo sapiens
AKGRQRGYYYDMGGYYVFDS

SEQ ID NO: 304 - protein, 6 aa, Homo sapiens
QSVSRY

SEQ ID NO: 305 - protein, 9 aa, Homo sapiens
QQTYSTFGP

SEQ ID NO: 306 - protein, 8 aa, Homo sapiens
GFTFSSAW

SEQ ID NO: 307 - protein, 10 aa, Homo sapiens
IKSKAAGGTA

SEQ ID NO: 308 - protein, 15 aa, Homo sapiens
TTGSLEGTITHAEDH

SEQ ID NO: 309 - protein, 6 aa, Homo sapiens
QSISSW

SEQ ID NO: 310 - protein, 9 aa, Homo sapiens
QQYNSYSLT

SEQ ID NO: 311 - protein, 8 aa, Homo sapiens
GFTFSNYA

SEQ ID NO: 312 - protein, 8 aa, Homo sapiens
ISSGGYNT

SEQ ID NO: 313 - protein, 11 aa, Homo sapiens
VTRRAGSYYAY

SEQ ID NO: 314 - protein, 11 aa, Homo sapiens
QSLAYSDGNTY

SEQ ID NO: 315 - protein, 3 aa, Homo sapiens
KVF

SEQ ID NO: 316 - protein, 9 aa, Homo sapiens
MQGTHWPIT

SEQ ID NO: 317 - protein, 8 aa, Homo sapiens
GYTFTNYY

SEQ ID NO: 318 - protein, 8 aa, Homo sapiens
INAGSGRT

SEQ ID NO: 319 - protein, 16 aa, Homo sapiens
ARGSAHIEAGGSAFDK

SEQ ID NO: 320 - protein, 11 aa, Homo sapiens
QSLIHSDGNIF

SEQ ID NO: 321 - protein, 3 aa, Homo sapiens
KVS

SEQ ID NO: 322 - protein, 8 aa, Homo sapiens
GFIFINYY

SEQ ID NO: 323 - protein, 8 aa, Homo sapiens
INPTSGKS

SEQ ID NO: 324 - protein, 16 aa, Homo sapiens
ARGSARAEAAGSAFDH

SEQ ID NO: 325 - protein, 6 aa, Homo sapiens
LDIKNY

SEQ ID NO: 326 - protein, 3 aa, Homo sapiens
DAS

SEQ ID NO: 327 - protein, 9 aa, Homo sapiens
QQYGKLPFT

SEQ ID NO: 328 - protein, 8 aa, Homo sapiens
GASMNGYY

SEQ ID NO: 329 - protein, 11 aa, Homo sapiens
IHYVGTTMKAT

SEQ ID NO: 330 - protein, 16 aa, Homo sapiens
ARRRYHFWSDPGTFDI

SEQ ID NO: 331 - protein, 8 aa, Homo sapiens
GINVGIYS

SEQ ID NO: 332 - protein, 7 aa, Homo sapiens
FKSDSDK

SEQ ID NO: 333 - protein, 9 aa, Homo sapiens
MMWHSSAWV

SEQ ID NO: 334 - protein, 8 aa, Homo sapiens
GFSFSTYA

SEQ ID NO: 335 - protein, 8 aa, Homo sapiens
ISGNGYSS

SEQ ID NO: 336 - protein, 14 aa, Homo sapiens
AKGDGGSGWFGFDY

SEQ ID NO: 337 - protein, 8 aa, Homo sapiens
NSNIGSYT

SEQ ID NO: 338 - protein, 3 aa, Homo sapiens
RSN

SEQ ID NO: 339 - protein, 11 aa, Homo sapiens
SAWDDSLNAYV

SEQ ID NO: 340 - protein, 8 aa, Homo sapiens
GYIFTTYY

SEQ ID NO: 341 - protein, 8 aa, Homo sapiens
INPAVGTT

SEQ ID NO: 342 - protein, 12 aa, Homo sapiens
ARELHATHFFDF

(19) United States
(12) Patent Application Publication
(10) Pub. No.: US 2022/0119502 A1
(43) Pub. Date: Apr. 21, 2022

(54) KAWASAKI DISEASE ANTIBODIES IDENTIFY HEPACIVIRUS PEPTIDES

(71) Applicants: Ann and Robert H. Lurie Children's Hospital of Chicago, Chicago, IL (US); Loyola University Chicago, Maywood, IL (US)

(72) Inventors: Anne H. Rowley, Oak Park, IL (US); Standford L. Shulman, Evanston, IL (US); Susan C. Baker, Elmhurst, IL (US)

(21) Appl. No.: 17/310,832

(22) PCT Filed: Feb. 28, 2020
(86) PCT No.: PCT/US20/20440

§ 371 Date: Aug. 25, 2021

Related U.S. Application Data
(60) Provisional application No. 62/811,930, filed on Feb. 28, 2019.

(51) Int. Cl. C07K 16/10; G01N 33/68; G01N 33/576

(52) U.S. Cl. CPC C07K 16/109; G01N 33/6893; G01N 33/5767; C07K 2317/565; G01N 2800/328; C07K 2317/40; C07K 2317/33; C07K 2317/34; C07K 2317/24

(57) ABSTRACT

The present disclosure provides monoclonal antibodies that target intracytoplasmic inclusion bodies and/or hepacivirus C NS4A and methods for their use.

### CROSS-REFERENCE TO RELATED APPLICATIONS

This application claims priority to U.S. Provisional Application No. 62/811,930 filed on Feb. 28, 2019, which is incorporated by reference in its entirety as if fully set forth herein.

### STATEMENT REGARDING FEDERALLY SPONSORED RESEARCH

This invention was made with government support under grant R21AI140029 awarded by the National Institutes of Health. The government has certain rights in this invention.

### REFERENCE TO A SEQUENCE LISTING SUBMITTED VIA EFS-WEB

The content of the ASCII text file of the sequence listing named “702581_01714_ST25.txt” which is 149 kb in size was created on Feb. 27, 2020 and electronically submitted via EFS-Web herewith the application is incorporated herein by reference in its entirety.

### BACKGROUND

Kawasaki Disease (KD) is a febrile illness of young childhood that has clinical and epidemiologic features of an infectious disease (1) including epidemics with geographic wavelike spread (2). KD can result in potentially severe or even fatal coronary artery aneurysms in infants and children (3). First described by Dr. Tomisaku Kawasaki in Japan in the 1960s, and now recognized worldwide, the etiology remains elusive. Diagnosis of KD, particularly of incomplete cases who can have prolonged fever but few other clinical manifestations, is a major clinical problem in pediatrics because potentially severe, lifelong sequelae can be reduced by timely administration of intravenous gammaglobulin (4). The Ann & Robert H. Lurie Children's Hospital of Chicago cares for 60 newly diagnosed children with KD each year, and most large US children's hospitals care for scores of newly diagnosed cases annually. High attack rates of KD are observed in Asian children, most likely because of genetic predisposition to the inciting agent (5). In Japan 1 in 65 children develop the disease by the age of 5 years (6).

The antigens triggering the immune response in KD patients have been unknown. Analysis of peripheral blood plasmablasts is emerging as a powerful tool in studies of pathogenesis, diagnosis, and therapeutic discovery in infectious diseases (7-15), vaccine science (15-21), and autoimmune disease (22, 23). Multiple studies have shown that >70% of peripheral blood plasmablasts express antibodies specific to the infectious or immunizing agent at 1-2 weeks following infection (24-27). Identification of specific KD antigens would enable diagnostic test development and improved therapies.

Applicants previously discovered an oligoclonal IgA response in KD arterial tissues (28), and made synthetic antibodies comprised of clonally expanded alpha heavy chains from KD arterial tissue paired with random light chains (29, 30). These antibodies identified intracytoplasmic inclusion bodies in KD but not in infant control ciliated bronchial epithelium by immunohistochemistry (29-31). However, these antibodies did not yield the specific antigen. A need remains in the art for further understanding of KD etiology, KD diagnostic methods, and additional KD treatment tools.

### SUMMARY OF THE DISCLOSURE

In a first aspect, provided herein is an isolated intracytoplasmic inclusion bodies (ICI) antibody or antigen binding fragment thereof comprising a heavy chain variable domain comprising a CDRH1 region selected from the group consisting of 272, 278, 284, 290, 295, 301, 306, 311, 317, 322, 328, 334, and 340; a CDRH2 region selected from the group consisting of 273, 279, 285, 291, 296, 302, 307, 312, 318, 323, 329, 335, and 341; and a CDRH3 region selected from the group consisting of 274, 280, 286, 299, 297, 303, 308, 313, 319, 324, 330, 336, and 342; and a light chain variable domain comprising a CDRL1 region selected from the group consisting of 269, 275, 281, 287, 293, 298, 304, 309, 314, 320, 325, 331, and 337; a CDRL2 region selected from the group consisting of 270, 276, 282, 288, 299, 315, 321, 326, 332, and 338; and a CDRL3 region selected from the group consisting of 271, 277, 283, 289, 294, 300, 305, 310, 316, 327, 333, and 339.

In some embodiments, the isolated antibody or antigen binding fragment thereof comprises (a) a heavy chain variable domain comprising a CDRH1 region consisting of SEQ ID NO:272, a CDRH2 region consisting of SEQ ID NO:273, and a CDRH3 region consisting of SEQ ID NO:274 and a light chain variable domain comprising a CDRL1 region consisting of SEQ ID NO:269, a CDRL2 region consisting of SEQ ID NO:270, and a CDRL3 region consisting of SEQ ID NO:271, or (b) a heavy chain variable domain comprising a CDRH1 region consisting of SEQ ID NO:278, a CDRH2 region consisting of SEQ ID NO:279, and a CDRH3 region consisting of SEQ ID NO:280 and a light chain variable domain comprising a CDRL1 region consisting of SEQ ID NO:275, a CDRL2 region consisting of SEQ ID NO:276, and a CDRL3 region consisting of SEQ ID NO:277, or (c) a heavy chain variable domain comprising a CDRH1 region consisting of SEQ ID NO:284, a CDRH2 region consisting of SEQ ID NO:285, and a CDRH3 region consisting of SEQ ID NO:286 and a light chain variable domain comprising a CDRL1 region consisting of SEQ ID NO:281, a CDRL2 region consisting of SEQ ID NO:282, and a CDRL3 region consisting of SEQ ID NO:283, or (d) a heavy chain variable domain comprising a CDRH1 region consisting of SEQ ID NO:290, a CDRH2 region consisting of SEQ ID NO:291, and a CDRH3 region consisting of SEQ ID NO:292 and a light chain variable domain comprising a CDRL1 region consisting of SEQ ID NO:287, a CDRL2 region consisting of SEQ ID NO:288, and a CDRL3 region consisting of SEQ ID NO:289, or (e) a heavy chain variable domain comprising a CDRH1 region consisting of SEQ ID NO:295, a CDRH2 region consisting of SEQ ID NO:296, and a CDRH3 region consisting of SEQ ID NO:297 and a light chain variable domain comprising a CDRL1 region consisting of SEQ ID NO:293, a CDRL2 region consisting of SEQ ID NO:282, and a CDRL3 region consisting of SEQ ID NO:294, or (f) a heavy chain variable domain comprising a CDRH1 region consisting of SEQ ID NO:301, a CDRH2 region consisting of SEQ ID NO:302, and a CDRH3 region consisting of SEQ ID NO:303 and a light chain variable domain comprising a CDRL1 region consisting of SEQ ID NO:298, a CDRL2 region consisting of SEQ ID NO:299, and a CDRL3 region consisting of SEQ ID NO:300, or (g) a heavy chain variable domain comprising a CDRH1 region consisting of SEQ ID NO:306, a CDRH2 region consisting of SEQ ID NO:307, and a CDRH3 region consisting of SEQ ID NO:308 and a light chain variable domain comprising a CDRL1 region consisting of SEQ ID NO:304, a CDRL2 region consisting of SEQ ID NO:282, and a CDRL3 region consisting of SEQ ID NO:305, or (h) a heavy chain variable domain comprising a CDRH1 region consisting of SEQ ID NO:311, a CDRH2 region consisting of SEQ ID NO:312, and a CDRH3 region consisting of SEQ ID NO:313 and a light chain variable domain comprising a CDRL1 region consisting of SEQ ID NO:309, a CDRL2 region consisting of SEQ ID NO:276, and a CDRL3 region consisting of SEQ ID NO:310, or (i) a heavy chain variable domain comprising a CDRH1 region consisting of SEQ ID NO:317, a CDRH2 region consisting of SEQ ID NO:318, and a CDRH3 region consisting of SEQ ID NO:319 and a light chain variable domain comprising a CDRL1 region consisting of SEQ ID NO:314, a CDRL2 region consisting of SEQ ID NO:315, and a CDRL3 region consisting of SEQ ID NO:316, or (j) a heavy chain variable domain comprising a CDRH1 region consisting of SEQ ID NO:322, a CDRH2 region consisting of SEQ ID NO:323, and a CDRH3 region consisting of SEQ ID NO:324 and a light chain variable domain comprising a CDRL1 region consisting of SEQ ID NO:320, a CDRL2 region consisting of SEQ ID NO:321, and a CDRL3 region consisting of SEQ ID NO:316, or (k) a heavy chain variable domain comprising a CDRH1 region consisting of SEQ ID NO:328, a CDRH2 region consisting of SEQ ID NO:329, and a CDRH3 region consisting of SEQ ID NO:330 and a light chain variable domain comprising a CDRL1 region consisting of SEQ ID NO:325, a CDRL2 region consisting of SEQ ID NO:326, and a CDRL3 region consisting of SEQ ID NO:327, or (1) a heavy chain variable domain comprising a CDRH1 region consisting of SEQ ID NO:334, a CDRH2 region consisting of SEQ ID NO:335, and a CDRH3 region consisting of SEQ ID NO:336 and a light chain variable domain comprising a CDRL1 region consisting of SEQ ID NO:331, a CDRL2 region consisting of SEQ ID NO:332, and a CDRL3 region consisting of SEQ ID NO:333, or (m) a heavy chain variable domain comprising a CDRH1 region consisting of SEQ ID NO:340, a CDRH2 region consisting of SEQ ID NO:341, and a CDRH3 region consisting of SEQ ID NO:342 and a light chain variable domain comprising a CDRL1 region consisting of SEQ ID NO:337, a CDRL2 region consisting of SEQ ID NO:338, and a CDRL3 region consisting of SEQ ID NO:339.

In a second aspect, provided herein is an isolated monoclonal antibody comprising (a) a light chain variable domain encoded by a sequence selected from the group consisting of SEQ ID NOs:165, 167, 169, 171, 173, 175, 177, 179, 181, 183, 185, 187, and 189; and (b) a heavy chain comprising a rabbit heavy chain constant domain and a heavy chain variable domain encoded by a sequence selected from the group consisting of SEQ ID NOs:166, 168, 170, 172, 174, 176, 178, 180, 182, 184, 186, 188, and 190.

In a third aspect, provided herein is an isolated monoclonal antibody comprising (a) a light chain variable domain encoded by a sequence selected from the group consisting of SEQ ID NOs:165, 167, 169, 171, 173, 175, 177, 179, 181, 183, 185, 187, and 189; and (b) a heavy chain variable domain encoded by a sequence selected from the group consisting of SEQ ID NOs:166, 168, 170, 172, 174, 176, 178, 180, 182, 184, 186, 188, and 190.

In some embodiments, the monoclonal antibody is biotinylated. In some embodiments, the antibody is a chimeric antibody and the heavy chain constant domain is from rabbit, mouse, rat, or nonhuman primate. In some embodiments, the light chain constant domain is a kappa light chain constant domain or a lambda light chain constant domain.

In some embodiments, the antibody includes the light chain variable region of SEQ ID NO:165 and the heavy chain variable region of SEQ ID NO:166, the light chain variable region of SEQ ID NO:167 and the heavy chain variable region of SEQ ID NO:168, the light chain variable region of SEQ ID NO:169 and the heavy chain variable region of SEQ ID NO:170, the light chain variable region of SEQ ID NO:171 and the heavy chain variable region of SEQ ID NO:172, the light chain variable region of SEQ ID NO:173 and the heavy chain variable region of SEQ ID NO:174, the light chain variable region of SEQ ID NO:175 and the heavy chain variable region of SEQ ID NO:176, the light chain variable region of SEQ ID NO:177 and the heavy chain variable region of SEQ ID NO:178, the light chain variable region of SEQ ID NO:179 and the heavy chain variable region of SEQ ID NO:180, the light chain variable region of SEQ ID NO:181 and the heavy chain variable region of SEQ ID NO:182, the light chain variable region of SEQ ID NO:183 and the heavy chain variable region of SEQ ID NO:184, the light chain variable region of SEQ ID NO:185 and the heavy chain variable region of SEQ ID NO:186, the light chain variable region of SEQ ID NO:187 and the heavy chain variable region of SEQ ID NO:188, or the light chain variable region of SEQ ID NO:189 and the heavy chain variable region of SEQ ID NO:190.

In some embodiments, the light chain variable domain is encoded by SEQ ID NO: 103 and the heavy chain variable domain is encoded by SEQ ID NO:102. In some embodiments, the light chain variable domain is encoded by SEQ ID NO:128 and the heavy chain variable domain is encoded by SEQ ID NO:127. In some embodiments, the isolated monoclonal antibody of claim 2, wherein the light chain variable domain is encoded by SEQ ID NO:150 and the heavy chain variable domain is encoded by SEQ ID NO:149. In some embodiments, the light chain variable domain is encoded by SEQ ID NO:122 and the heavy chain variable domain is encoded by SEQ ID NO:121. In some embodiments, the light chain variable domain is encoded by SEQ ID NO:148 and the heavy chain variable domain is encoded by SEQ ID NO:147. In some embodiments, the light chain variable domain is encoded by SEQ ID NO:120 and the heavy chain variable domain is encoded by SEQ ID NO:119. In some embodiments, the light chain variable domain is encoded by SEQ ID NO:34 and the heavy chain variable domain is encoded by SEQ ID NO:33. In some embodiments, the light chain variable domain is encoded by SEQ ID NO:138 and the heavy chain variable domain is encoded by SEQ ID NO:137. In some embodiments, the light chain variable domain is encoded by SEQ ID NO:63 and the heavy chain variable domain is encoded by SEQ ID NO:62. In some embodiments, the light chain variable domain is encoded by SEQ ID NO:67 and the heavy chain variable domain is encoded by SEQ ID NO:66. In some embodiments, the light chain variable domain is encoded by SEQ ID NO:77 and the heavy chain variable domain is encoded by SEQ ID NO:76. In some embodiments, the light chain variable domain is encoded by SEQ ID NO:144 and the heavy chain variable domain is encoded by SEQ ID NO:143. In some embodiments, the light chain variable domain has a sequence encoded by SEQ ID NO:160 and the heavy chain variable domain has a sequence encoded by SEQ ID NO:159.

In a fourth aspect, provided herein is a method of diagnosing Kawasaki Disease in a subject, comprising the steps of i) obtaining a sample from a subject suspected of having Kawasaki Disease; ii) contacting the sample with an antibody as described herein; and ii) detecting the binding of the antibody in the sample, whereby binding of the antibody indicates the presence of Kawasaki Disease. In some embodiments, the sample is a blood sample. In some embodiments, detecting the binding of the antibody in the sample is carried out using ELISA, Western blot, immunostaining, immunoprecipitation, flow cytometry, sensor chips, or magnetic beads.

In a fifth aspect, provided herein is a method of detecting intracytoplasmic inclusion bodies in a subject, comprising the steps of i) obtaining a sample from a subject suspected of having Kawasaki Disease; ii) contacting the sample with an antibody as described herein; and ii) detecting the binding of the antibody in the sample, whereby binding of the antibody indicates the presence of intracytoplasmic inclusion bodies.

In a sixth aspect, provided herein is a method of detecting hepacivirus C in a subject, comprising the steps of: i) obtaining a sample form a subject suspected of having a hepacivirus C infection; ii) contacting the sample with the antibody of any of claims 1-31; and iii) detecting the binding of the antibody in the sample, wherein binding of the antibody indicates the presence of hepacivirus C infection.

### BRIEF DESCRIPTION OF DRAWINGS

The patent or patent application file contains at least one drawing in color. Copies of this patent or patent application publication with color drawings will be provided by the Office upon request and payment of the necessary fee.

FIGS. 1A-1D show characteristics of plasmablasts (PB) isolated from peripheral blood of 11 children 1-3 weeks after KD fever onset. (A) Study overview. (B to D) Analysis of single cells from 11 children with KD. (B) Most PB were VH3, VH4, or VH1. (C) IgA and IgG PB were most commonly identified. (D) Of 1156 PB sequenced, 42 sets of oligoclonal PB were identified for antibody production, and 15 somatically mutated IgA plasmablasts were also arbitrarily selected for production. Of 60 monoclonal antibodies prepared, 10 strongly bound to intracytoplasmic inclusion bodies (ICI) in ciliated bronchial epithelial cells of children who died of KD using immunohistochemistry, and 22 were found to bind weakly. Strongly binding antibodies were tested on an animal virus peptide array.

FIGS. 2A-2F show intracytoplasmic inclusion bodies (ICI) are detected in KD ciliated bronchial epithelium by immunohistochemistry using KD monoclonal antibodies. (A, B, E, F; ICI are brown and are indicated with arrows). (A) ICI are identified by monoclonal antibody KD1-2G11 in a 4-month-old Caucasian infant male (KD patient A) who died of acute KD in the US at 3 weeks after fever onset. (B) ICI are identified by KD1-2G11 in a 9-month-old Japanese infant male who died of acute KD in Japan on day 18 after fever onset. (C) No staining is observed using KD1-2G11 in a 3-month-old US female infant who died of influenza. (D) No staining is observed using control synthetic antibody in KD patient A. (E) ICI are identified in KD patient A using antibody KD4-2H4; (F) ICI are identified in KD patient A using antibody KD7-1D3. A-F, 20× objective. Assays performed in duplicate.

FIGS. 3A-3C show Animal Virus Peptide Array Analysis and Discovered Motifs of Monoclonal Antibody KD4-2H4, and Epitope Mapping of Monoclonal Antibodies KD4-2H4. (A) Animal virus peptide array demonstrates that KD4-2H4 recognizes multiple related peptides of hepacivirus C NS4A with averaged median foreground fluorescence intensities above a cutoff value of 200; the epitope ID from the Immune Epitope Database (available on the World Wide Web at iedb.org) is listed for each reacting peptide, (B) Three motifs of KD4-2H4 binding are identified as statistically significant by MEME bioinformatics analysis (motifs 1 and 3 are partially overlapping), (C) Substitution matrix array of peptide AIIPDREALYQEFDEME (SEQ ID NO:219) using KD4-2H4, Preferred amino acids for binding are shown in red (left), and non-preferred amino acid substitutions shown in blue (right).

FIGS. 4A-4B show additional KD monoclonal antibodies recognize KD peptide. (A) KD monoclonal antibodies bind to KD peptide by ELISA. The OD reading of a scrambled version of the peptide was subtracted as a negative control. Antibodies KD4-2H4 and KD6-2B2 bind most strongly to the peptide, followed by KD8-1D4, KD8-1B10, and KD6-1A10. Samples were assayed in triplicate at each dilution of 10, 1, and 0.1 μg/ml. Dots represent individual assay results and horizontal lines represent mean of three assays. (B) Substitution matrix array of peptide AVIPDREALYQDIDEME (SEQ ID NO:212, derived from KD peptide) using KD6-2B2 demonstrates that KD6-2B2 shares an epitope with KD4-2H4. Preferred amino acids for binding are shown in red (left), and non-preferred amino acid substitutions shown in blue (right).

FIGS. 5A-5D show KD peptide blocks binding of KD monoclonal antibodies to KD intracytoplasmic inclusion bodies (ICI). Ciliated bronchial epithelium of KD patient A; ICI stain brown (arrows). (A) ICI are identified in KD patient A using antibody KD4-2H4, preincubated with scrambled peptide; (B) ICI staining is blocked when antibody KD4-2H4 is pre-incubated with KD peptide. (C) ICI are identified in KD patient A using antibody KD6-2B2, preincubated with scrambled peptide; (B) ICI staining is blocked when antibody KD6-2B2 is pre-incubated with KD peptide. A-D, 20× objective. Assays were performed in duplicate.

FIG. 6 shows Western blot analysis of sera for reactivity to KD peptide fusion protein. For each blot, lane 1 contains GST alone, lane 2 contains GST-KD peptide fusion protein (GST-3X), and lane 3 contains human IgG as a positive control. Blots were stripped and polyclonal anti-GST antibody was applied to ensure that the GST fusion proteins were present, each serum sample was tested 2-5 times, and a representative blot is shown. FC=febrile control, IC=infant control. M=All Blue Standard (Biorad 1610373). Molecular weight of IgG heavy chain is 50 kD, GST-3X is ˜35 kD, and GST alone is ˜26 kD (arrows).

FIG. 7 shows KD monoclonal antibodies that recognize NSA peptide 2 are not polyreactive. ELISA of 5 monoclonal KD antibodies (KD4-2H4, KD6-2B2, KD8-1D4, KD8-1B10, and KD8-1A10) that recognize NS4A peptide 2 epitope demonstrates a lack of reactivity of the antibodies to single stranded DNA, insulin, and bovine serum albumin. Polyreactive VH4-34 antibody KD11-1C1 is used as a positive control. Assays performed in triplicate.

FIG. 8A-8C show ITM2B is not the KD antigen in inclusion bodies. Immunohistochemistry of ciliated bronchial epithelium on lung tissue from KD patient A. A) Staining with biotinylated human monoclonal antibody KD4-2H4 demonstrates inclusion bodies (arrows), B) Staining with rabbit polyclonal antibody to integral membrane protein 2B (ITM2B) reveals patchy staining of the bronchial epithelium, C) Staining with biotinylated human KD4-2H4 following incubation with rabbit polyclonal ITM2B antibody reveals that ITM2B antibody does not block binding of KD4-2H4 to inclusion bodies. Images taken with 20× objective.

FIG. 9 shows a general outline of KD monoclonal antibody preparation from peripheral blood plasmablasts.

### DETAILED DESCRIPTION OF THE INVENTION

The present disclosure describes monoclonal antibodies that can bind to intracytoplasmic inclusion bodies (ICI) and/or hepacivirus C NS4A. The present disclosure also describes methods of using the disclosed monoclonal antibodies to diagnose and treat Kawasaki Disease (KD) in a subject.

The terms “antibody” or “antibody molecule” are used herein interchangeably and refer to immunoglobulin molecules or other molecules which comprise an antigen binding domain. The term “antibody” or “antibody molecule” as used herein is thus intended to include whole antibodies (e.g., IgG, IgA, IgE, IgM, or IgD), monoclonal antibodies, chimeric antibodies, humanized antibodies, and antibody fragments, including single chain variable fragments (ScFv), single domain antibody, and antigen-binding fragments, genetically engineered antibodies, among others, as long as the characteristic properties (e.g., ability to bind CD30) are retained. The term “antibody fragment” as used herein is intended to include any appropriate antibody fragment that displays antigen binding function, for example, Fab, Fab′, F(ab′)2, scFv, Fv, dsFv, ds-scFv, Fd, mini bodies, monobodies, and multimers thereof and bispecific antibody fragments.

As stated above, the term “antibody” includes “antibody fragments” or “antibody-derived fragments” and “antigen binding fragments” which comprise an antigen binding domain. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), (see for instance Bird et al., Science 242, 423-426 (1988) and Huston et al., PNAS USA 85, 5879-5883 (1988)). Such single chain antibodies are encompassed within the term antibody unless otherwise noted or clearly indicated by context.

Antibodies can be genetically engineered from the CDRs and monoclonal antibody sequences described herein into antibodies and antibody fragments by using conventional techniques such as, for example, synthesis by recombinant techniques or chemical synthesis. Techniques for producing antibody fragments are well known and described in the art.

One may wish to engraft one or more CDRs from the monoclonal antibodies described herein into alternate scaffolds. For example, standard molecular biological techniques can be used to transfer the DNA sequences encoding the antibody's CDR(s) to (1) full IgG scaffold of human or other species; (2) a scFv scaffold of human or other species, or (3) other specialty vectors. If the CDR(s) have been transferred to a new scaffold all of the previous modifications described can also be performed. For example, one could consult Biotechnol Genet Eng Rev, 2013, 29:175-86 for a review of useful methods.

The antibodies or antibody fragments can be wholly or partially synthetically produced. Thus the antibody may be from any appropriate source, for example recombinant sources and/or produced in transgenic animals or transgenic plants. Thus, the antibody molecules can be produced in vitro or in vivo. The antibody or antibody fragment can be made that comprises all or a portion of a heavy chain constant region, such as an IgG1, IgG2, IgG3, IgG4, IgA1, IgA2, IgE, IgM or IgD constant region.

Furthermore, the antibody or antibody fragment can comprise all or a portion of a kappa light chain constant region or a lambda light chain constant region. All or part of such constant regions may be produced wholly or partially synthetic. Appropriate sequences for such constant regions are well known and documented in the art.

The term “fragment” as used herein refers to fragments of biological relevance (functional fragment), e.g., fragments which can contribute to or enable antigen binding, e.g., form part or all of the antigen binding site or can contribute to the prevention of the antigen interacting with its natural ligands. Fragments in some embodiments comprise a heavy chain variable region (VH domain) and light chain variable region (VL) of the disclosure. In some embodiments, the fragments comprise one or more of the heavy chain complementarity determining regions (CDRHs) of the antibodies or of the VH domains, and one or more of the light chain complementarity determining regions (CDRLs), or VL domains to form the antigen binding site.

The term “complementarity determining regions” or “CDRs,” as used herein, refers to part of the variable chains in immunoglobulins (antibodies) and T cell receptors, generated by B-cells and T-cells respectively, where these molecules bind to their specific antigen. As the most variable parts of the molecules, CDRs are crucial to the diversity of antigen specificities generated by lymphocytes. There are three CDRs (CDR1, CDR2 and CDR3), arranged non-consecutively, on the amino acid sequence of a variable domain of an antigen binding site. Since the antigen binding sites are typically composed of two variable domains (on two different polypeptide chains, heavy and light chain), there are six CDRs for each antigen binding site that can collectively come into contact with the antigen. A single whole antibody molecule has two antigen binding sites and therefore contains twelve CDRs. Sixty CDRs can be found on a pentameric IgM molecule.

Within the variable domain, CDR1 and CDR2 may be found in the variable (V) region of a polypeptide chain, and CDR3 includes some of V, and all of diversity (D, heavy chains only) and joining (J) regions. Since most sequence variation associated with immunoglobulins and T cell receptors is found in the CDRs, these regions are sometimes referred to as hypervariable regions. Among these, CDR3 shows the greatest variability as it is encoded by a recombination of VJ in the case of a light chain region and VDJ in the case of heavy chain regions. The tertiary structure of an antibody is important to analyze and design new antibodies.

As used herein, the terms “proteins” and “polypeptides” are used interchangeably herein to designate a series of amino acid residues connected to the other by peptide bonds between the alpha-amino and carboxy groups of adjacent residues. The terms “protein” and “polypeptide” refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing. The antibodies of the present invention are polypeptides, as well the antigen-binding fragments and fragments thereof.

The terms “monoclonal antibody” or “monoclonal antibody composition” as used herein refer to a preparation of antibody molecules of a single amino acid composition that specifically binds to a single epitope of the antigen.

The term “chimeric antibody” refers to an antibody comprising a variable region, i.e., binding region, from one source or species and at least a portion of a constant region derived from a different source or species, usually prepared by recombinant DNA techniques. Other forms of “chimeric antibodies” are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. In some embodiments, the antibodies are chimeric antibodies including heavy chain constant domains from non-human mammals (e.g., mouse, rat, rabbit, or non-human primate). In some embodiments, the antibodies disclosed in the present invention are chimeric antibodies including constant regions from rabbit heavy chain immunoglobulin sequences. Suitable heavy chain constant region sequences from non-human mammals, including mouse, rat, rabbit, and non-human primate are known in the art.

The antibodies disclosed in the present invention are human antibodies, as they include the constant region from human germline immunoglobulin sequences. The term “recombinant human antibody” includes all human antibodies that are prepared, expressed, created, or isolated by recombinant means, such as antibodies isolated from a host cell such as an SP2-0, NS0 or CHO cell (like CHO Kl) or from an animal (e.g., a mouse) that is transgenic for human immunoglobulin genes or antibodies expressed using a recombinant expression vector transfected into a host cell. Such recombinant human antibodies have variable and in some embodiments, constant regions derived from human germline immunoglobulin sequences in a rearranged form.

ICI and KD specific monoclonal antibodies described herein include the following (Tables 1A and 1B). The sequences referenced in Table 1B are nucleotide sequences, whereby the nucleotide sequence encodes for the amino acid sequence of the light or heavy chain variable region.

TABLE 1A
Heavy and Light Chain Variable Region nucleotide sequences, including CDR1, CDR2, and CDR3.

| | | Nucleotide Sequence | | Nucleotide Sequence |
|---|---|---|---|---|
| Antibody | Light Chain | Encoding the Light | Heavy Chain | Encoding the Heavy |
| name | Variable Region | Chain Variable Region | Variable Region | Chain Variable Region |
| KD4-2H4 | QSALTQPRSVS | CAGTCTGCCCTGACTCAGCCTCGCT | EVQLVESGGGLV | GAGGTGCAGCTGGTGGAGTCCGG |
| | GSPGQSVTISCT | CAGTGTCCGGGTCTCCTGGACAGTC | QPGGSLRLSCAA | GGGAGGCTTAGTTCAGCCTGGGGG |
| | GTSSDVGGYNY | AGTCACCATCTCCTGCACTGGAACC | SGFTFSSYWMYW | GTCCCTGAGACTCTCCTGTGCAGC |
| | VSWYQQHPGK | AGCAGTGATGTTGGTGGTTATAACT | VRQAPGKGLVW | CTCTGGATTCACCTTCAGTAGCTA |
| | APKLMIYDVSK | ATGTCTCCTGGTACCAACAGCACCC | VSRINSDGSTTNY | CTGGATGTACTGGGTCCGCCAAGC |
| | RPSGVPDRFSG | AGGCAAAGCCCCCAAACTCATGATT | ADSVKGRFTISRD | TCCAGGGAAGGGGCTGGTGTGGG |
| | SKSGNTASLTIS | TATGATGTCAGTAAGCGGCCCTCAG | NAKNTLYLQMNS | TCTCACGTATTAATAGTGATGGGA |
| | GLQAEDEADY | GGGTCCCTGATCGCTTCTCTGGCTCC | LRAEDTAVYFCA | GTACCACGAACTACGCGGACTCCG |
| | YCCSYAGSYTL | AAGTCTGGCAACACGGCCTCCCTGA | RYAHLGIGWYF | TGAAGGGCCGATTCACCATCTCCA |
| | LFGGGTKLTVL | CCATCTCTGGGCTCCAGGCTGAGGA | DLWGRGTLVTVS | GAGACAACGCCAAGAACACGCTG |
| | (SEQ ID NO: 165; | TGAGGCTGATTATTACTGCTGCTCA | S | TATCTGCAAATGAACAGTCTGAGA |
| | CDR1 SEQ ID | TATGCAGGCAGCTACACTTTGTTAT | (SEQ ID NO: 166; | GCCGAGGACACGGCTGTGTATTTC |
| | NO: 269; CDR2 | TCGGCGGAGGGACCAAGCTGACCGT | CDR1 SEQ ID | TGTGCAAGATATGCCCACCTGGGG |
| | SEQ ID NO: 270; | CCTAG | NO: 272; CDR2 | ATAGGTTGGTACTTCGATCTCTGG |
| | CDR3 SEQ ID | (SEQ ID NO: 103) | SEQ ID NO: 273; | GGCCGTGGCACCCTGGTCACCGTC |
| | NO: 271) | | CDR3 SEQ ID | TCCTCAG |
| | | | NO: 274) | (SEQ ID NO: 102) |
| KD6-2B2 | DIQMTQSPSTLS | GACATCCAGATGACCCAGTCTCCTT | EVQLVESGGGVA | GAGGTGCAGCTGGTGGAGTCTGG |
| | ASAGDRVTITC | CCACCCTGTCTGCATCTGCAGGAGA | QPGTSLRLSCEAS | GGGAGGCGTGGCCCAGCCTGGGA |
| | RASQSVSKYLA | CAGAGTCACCATCACTTGCCGGGCC | GFTFRDYAMRW | CGTCTCTGAGACTCTCCTGTGAAG |
| | WYQQKPGKAP | AGTCAGAGTGTTAGTAAGTACTTGG | VRQAPGKGLEW | CGTCTGGATTCACCTTCCGTGACT |
| | RLLIYKASSLQS | CCTGGTATCAGCAGAAACCAGGGA | VAFIWNDGSKKY | ATGCCATGCGCTGGGTCCGCCAGG |
| | GVPSRFSGSGS | AAGCCCCTAGGCTCCTGATCTATAA | YTDSVRGRFTISR | CTCCAGGCAAGGGGCTGGAGTGG |
| | GTEFTLTISSLQ | GGCATCTAGTTTACAAAGTGGGGTC | DNSKNMLYLQM | GTGGCATTTATATGGAATGATGGA |
| | PDDFATYYCQ | CCATCAAGGTTCAGTGGCAGTGGAT | DSLRAEDTALYY | AGTAAGAAATATTACACAGACTCC |
| | HYNTYPSWTF | CTGGGACAGAATTCACTCTCACCAT | CARKMSEDDAF | GTGAGGGGCCGATTCACCATCTCC |
| | GQGTKVEIK | CAGCAGCCTGCAGCCTGATGATTTT | DLWGQGTMVTV | AGAGACAATTCCAAGAACATGCT |
| | (SEQ ID NO: 167; | GCTACTTATTACTGCCAACACTATA | (SEQ ID NO: 168; | GTATCTGCAAATGGACAGCCTGAG |
| | CDR1 SEQ ID | ATACTTATCCTTCGTGGACGTTCGG | CDR1 SEQ ID | AGCCGAGGACACGGCTCTTTATTA |
| | NO: 275; CDR2 | CCAAGGGACCAAGGTGGAAATCAA | NO: 278; CDR2 | CTGTGCCAGAAAGATGAGTGAAG |
| | SEQ ID NO: 276; | A | SEQ ID NO: 279; | ATGATGCTTTTGATCTGTGGGGCC |
| | CDR3 SEQ ID | (SEQ ID NO: 128) | CDR3 SEQ ID | AAGGGACAATGGTCACCGTCT |
| | NO: 277) | | NO: 280) | (SEQ ID NO: 127) |
| KD8-1D4 | DIQMTQSPSSLS | GACATCCAGATGACCCAGTCTCCAT | EVQLVESGGGLV | GAGGTGCAGCTGGTGGAGTCTGG |
| | ASVGDRVTITC | CCTCCCTGTCTGCATCCGTAGGAGA | QPGGSLRLSCAA | GGGAGGCTTGGTCCAGCCTGGAG |
| | RASQGIRNDLG | CAGAGTCACCATCACTTGCCGGGCA | SGFTFSDYYMDW | GGTCCCTGAGACTCTCCTGTGCAG |
| | WYQQKPGKAP | AGTCAGGGCATTAGAAATGATTTAG | VRQAPGKGLEW | CCTCTGGATTCACCTTCAGTGACT |
| | KLLIYGASSLQS | GCTGGTATCAGCAGAAACCAGGGA | VGRTRNKANSYTT | ACTACATGGACTGGGTCCGCCAGG |
| | GVPSRFSGSGS | AAGCCCCTAAGCTCCTGATCTATGG | EYAASVKGRFTIS | CTCCAGGGAAGGGGCTGGAGTGG |
| | GTDFTLTISSLQ | TGCATCCAGTTTACAAAGTGGGGTC | RDDSKNSLYLQM | GTTGGCCGCACTAGAAACAAAGCT |
| | PEDIATYYCLQ | CCATCAAGATTCAGCGGCAGTGGAT | NSLKTEDTAVYY | AACAGTTACACCACAGAATACGCC |
| | EYTYPLTFGGG | CTGGCACAGATTTCACTCTCACCAT | CARFLLVADAFD | GCGTCTGTGAAAGGCAGATTCACC |
| | TKVEIK | CAGCAGCCTGCAGCCTGAAGATATT | IWGQGTMVTV | ATCTCAAGAGATGATTCAAAGAAC |
| | (SEQ ID NO: 169; | GCAACCTATTACTGTCTACAAGAAT | (SEQ ID NO: 170; | TCACTGTATCTGCAAATGAACAGC |
| | CDR1 SEQ ID | ACACTTACCCGCTCACTTTCGGCGG | CDR1 SEQ ID | CTGAAAACCGAGGACACGGCCGT |
| | NO: 281; CDR2 | AGGGACCAAGGTGGAAATCAAA | NO: 284; CDR2 | GTATTACTGTGCTAGATTTCTGCT |
| | SEQ ID NO: 282; | (SEQ ID NO: 150) | SEQ ID NO: 285; | GGTTGCGGATGCTTTTGATATCTG |
| | CDR3 SEQ ID | | CDR3 SEQ ID | GGGCCAAGGGACAATGGTCACCG |
| | NO: 283) | | NO: 286) | TCT |
| | | | | (SEQ ID NO: 149) |
| KD6-1A2 | QTVVTQEPSLT | CAGACTGTGGTGACTCAGGAGCCCT | EVQLVQSGAEVK | GAGGTGCAGCTGGTGCAGTCTGGG |
| | VSPGETVTLTC | CACTGACTGTGTCCCCAGGAGAGAC | KTGSSVRLSCKTS | GCTGAGGTGAAGAAGACTGGGTC |
| | GSNSGAVTSGH | AGTCACTCTCACCTGTGGCTCCAAC | GYTFTYRYLHWV | CTCAGTGAGGCTTTCCTGCAAGAC |
| | HPHWFQQKPG | AGTGGAGCTGTCACCAGTGGTCATC | RQAPGHALEWLG | CTCCGGATACACCTTCACATACCG |
| | QAPRTLIYDTTN | ATCCCCACTGGTTCCAGCAGAAGCC | WITIYNGNTKYAQ | CTACCTGCACTGGGTGCGACAGGC |
| | KVSWTPARFSA | TGGCCAAGCACCCAGGACTCTCATT | EFQDRVTITREMS | CCCCGGACACGCGCTTGAGTGGCT |
| | SLLGGKAALTL | TATGACACAACCAACAAGGTCTCCT | LTAVYMELRSLR | GGGGTGGATTACGATTTATAATGG |
| | SGAQPEDEADY | GGACACCTGCCCGCTTCTCAGCCTC | SEDTAMYYCARS | CAACACCAAGTATGCACAGGAATT |
| | YCLVSYSGGR | CCTCCTTGGGGGCAAAGCTGCCCTG | ALSGDNAFEFW | CCAGGACAGAGTCACCATTACCAG |
| | MVFGGGTKLT | ACCCTTTCGGGTGCGCAGCCTGAGG | AQGTMVTV | GGAAATGTCCTTGACCGCAGTATA |
| | VL | ATGAGGCTGACTACTATTGCTTGGT | (SEQ ID NO: 172; | CATGGAGTTGAGGAGCCTAAGATC |
| | (SEQ ID NO: 171; | CTCCTATAGTGGTGGTCGTATGGTC | CDR1 SEQ ID | TGAGGACACAGCCATGTATTACTG |
| | CDR1 SEQ ID | TTCGGCGGAGGGACCAAGCTGACCG | NO: 290; CDR2 | TGCAAGGTCCGCGTTGTCTGGCGA |
| | NO: 287; CDR2 | TCCTAC | SEQ ID NO: 291; | TAATGCCTTTGAATTCTGGGCCCA |
| | SEQ ID NO: 288; | (SEQ ID NO: 122) | CDR3 SEQ ID | AGGGACAATGGTCACCGTCT |
| | CDR3 SEQ ID | | NO: 292) | (SEQ ID NO: 121) |
| | NO: 289) | | | |
| KD8-1B10 | DIQMTQSPSSLS | GACATCCAGATGACCCAGTCTCCAT | EVQLVESGGGLV | GAGGTGCAGCTGGTGGAGTCTGG |
| | ASLGDRVTITC | CCTCCCTGTCTGCATCTTTAGGAGA | QPGGSLRLSCAA | GGGAGGCTTGGTCCAGCCTGGAG |
| | RASQGIRHDLG | CAGAGTCACCATCACTTGCCGGGCA | SGFTFSDHYMDW | GGTCCCTGAGACTCTCCTGTGCAG |
| | WYQQKPGKAP | AGTCAGGGCATTAGACATGATTTAG | VRQVPRKGLEW | CCTCTGGATTCACCTTCAGTGACC |
| | KLLIYGASILQT | GCTGGTATCAGCAGAAACCAGGGA | VGRARNKANSYTT | ACTATATGGACTGGGTCCGCCAGG |
| | GVPSRFSGSGS | AAGCCCCTAAGCTCCTCATCTATGG | EYAASVKGRFTIS | TTCCAAGGAAGGGGCTGGAGTGG |
| | GTDFTLTISSLQ | TGCATCCACTTTACAAACTGGGGTC | RDDSKNSLYLQM | GTTGGCCGTGCTAGAAACAAAGCT |
| | PEDFATYYCLQ | CCATCAAGGTTCAGCGGCAGTGGGT | NSLKSEDTAVYY | AACAGTTACACCACAGAATACGCC |
| | KYNYPLTFGG | CTGGCACAGATTTCACTCTCACCAT | CARFLLIADAFDI | GCGTCTGTGAAAGGCAGATTCACC |
| | GTKVEIK | CAGCAGCCTGCAGCCTGAAGATTTT | WGQGTMVTV | ATCTCAAGAGATGATTCAAAGAAC |
| | (SEQ ID NO: 173; | GCAACTTATTACTGTCTACAAAAGT | (SEQ ID NO: 174; | TCACTGTATCTACAAATGAACAGC |
| | CDR1 SEQ ID | ACAACTACCCGCTCACTTTCGGCGG | CDR1 SEQ ID | CTGAAAAGCGAGGACACGGCCGT |
| | NO: 293; CDR2 | AGGGACCAAGGTGGAGATCAAA | NO: 295; CDR2 | GTATTATTGTGCTAGATTTCTGCTT |
| | SEQ ID NO: 282; | (SEQ ID NO: 148) | SEQ ID NO: 296; | ATTGCGGATGCTTTTGATATCTGG |
| | CDR3 SEQ ID | | CDR3 SEQ ID | GGCCAAGGGACAATGGTCACCGT |
| | NO: 294) | | NO: 297) | CT |
| | | | | (SEQ ID NO: 147) |
| KD6-1A10 | QSVLTQPPSAS | CAGTCTGTGCTGACGCAGCCACCCT | VQLVESGGGVVQ | GTGCAGCTGGTGGAGTCGGGGGG |
| | GTPGQGVTISCS | CAGCGTCTGGGACCCCCGGGCAGGG | PGRSLRLSCAASG | AGGCGTGGTCCAGCCTGGGAGGTC |
| | GSRSNIGSNTV | CGTCACCATCTCTTGTTCTGGAAGC | FSFSNYFMHWVR | CCTGAGACTCTCCTGTGCAGCCTC |
| | NWYRQFPGTAP | AGGTCCAACATCGGAAGTAATACTG | HVPGKGLEWLAT | TGGATTCAGCTTCAGTAACTATTT |
| | KLLIYKNNQRPS | TTAACTGGTACCGGCAGTTCCCAGG | IWYDGSDKYYVD | CATGCACTGGGTCCGCCATGTTCC |
| | GVPDRFSGSKP | AACGGCCCCCAAACTCCTCATCTAT | SVKGRFTISRDNS | AGGCAAGGGGCTGGAGTGGCTGG |
| | GTSAYLAITGL | AAGAACAATCAGCGGCCCTCAGGG | KNTLYLQMGSLR | CAACCATCTGGTATGATGGAAGTG |
| | QSDDQADYYC | GTCCCTGACCGATTCTCTGGCTCCA | AEDTAMYYCAK | ATAAATACTATGTAGACTCCGTGA |
| | AAWDDSLKGV | AGCCTGGCACCTCAGCCTACCTGGC | GRQRGYYYDM | AGGGCCGATTCACCATCTCCAGAG |
| | VFGGGTKLTVL | CATCACTGGGCTCCAGTCTGACGAT | GGYYVFDSWGQ | ACAACTCCAAGAACACGCTCTATC |
| | (SEQ ID NO: 175; | CAGGCTGATTATTACTGTGCAGCAT | GTLVTVSS | TACAAATGGGCAGCCTGAGAGCT |
| | CDR1 SEQ ID | GGGATGACAGCCTGAAAGGTGTGGT | (SEQ ID NO: 176; | GAGGACACGGCTATGTATTACTGT |
| | NO: 298; CDR2 | GTTCGGCGGAGGGACCAAGCTGACC | CDR1 SEQ ID | GCGAAAGGGAGACAACGAGGATA |
| | SEQ ID NO: 299; | GTCCTAA | NO: 301; CDR2 | TTACTACGATATGGGTGGTTATTA |
| | CDR3 SEQ ID | (SEQ ID NO: 120) | SEQ ID NO: 302; | TGTTTTTGACTCCTGGGGCCAGGG |
| | NO: 300) | | CDR3 SEQ ID | AACCCTGGTCACCGTCTCCTCAG |
| | | | NO: 303) | (SEQ ID NO: 119) |
| KD1-2G11 | DIQMTQSPSSLS | GACATCCAGATGACCCAGTCTCCAT | EVQLVESGGGLV | GAGGTGCAGCTGGTGGAGTCTGG |
| | ASVGDRVTITC | CCTCCCTGTCTGCATCTGTGGGGGA | KPGGSLRLSCAA | GGGAGGCTTGGTAAAGCCTGGGG |
| | RASQSVSRYLN | CAGAGTCACCATCACTTGCCGGGCA | SGFTFSSAWLSW | GGTCCCTTAGACTCTCCTGTGCAG |
| | WYQQKPGRAP | AGTCAGAGCGTTAGCAGGTATCTAA | VRQDPGKGLEWL | CCTCTGGATTCACTTTCAGTAGCG |
| | RLLIFGASSLRS | ATTGGTATCAGCAGAAACCAGGGA | GRIKSKAAGGTAD | CCTGGTTGAGCTGGGTCCGTCAGG |
| | GVPSRFSGSGS | GAGCCCCTAGGCTCCTGATCTTTGG | HAASVKGRFTISR | ATCCAGGGAAGGGGCTGGAGTGG |
| | GTDFSLTINSLQ | TGCATCCAGTTTACGTAGTGGAGTC | DDSKNTLYLQMN | CTTGGCCGTATTAAAAGTAAAGCT |
| | PEDFATYYCQQ | CCATCCAGGTTCAGTGGCAGTGGAT | SLKIEDTAVYYC | GCTGGTGGGACAGCAGACCACGC |
| | TYSTFGPFGPG | CTGGGACAGATTTCTCTCTCACCAT | TTGSLEGTITHA | TGCATCCGTGAAAGGCAGATTCAC |
| | TKVDIK | CAACAGTCTGCAACCTGAAGATTTT | EDHWGQGTLVT | CATCTCAAGAGATGATTCAAAAAA |
| | (SEQ ID NO: 177; | GCAACTTACTACTGTCAACAGACTT | VSS | CACGTTATATCTGCAAATGAACAG |
| | CDR1 SEQ ID | ACAGTACTTTCGGACCTTTCGGCCC | (SEQ ID NO: 178; | CCTGAAAATCGAGGACACAGCCG |
| | NO: 304; CDR2 | TGGGACCAAAGTGGATATCAAA | CDR1 SEQ ID | TCTATTACTGTACCACAGGGTCAT |
| | SEQ ID NO: 282; | (SEQ ID NO: 34) | NO: 306; CDR2 | TGGAGGGTACAATTACACACGCG |
| | CDR3 SEQ ID | | SEQ ID NO: 307; | GAGGACCACTGGGGCCAGGGAAC |
| | NO: 305) | | CDR3 SEQ ID | CCTGGTCACCGTCTCCTCA |
| | | | NO: 308) | (SEQ ID NO: 33) |
| KD7-1D3 | DIQMTQSPSTLS | GACATCCAGATGACCCAGTCTCCTT | EVQLLESGGGLV | GAGGTGCAGCTGTTGGAGTCTGGG |
| | ASVGERVTITC | CCACCCTGTCTGCATCTGTAGGAGA | QPAGSLRLACAA | GGAGGCCTGGTACAGCCGGCGGG |
| | RASQSISSWLA | GAGAGTCACCATCACTTGCCGGGCC | SGFTFSNYAMTW | GTCCCTGAGACTCGCCTGTGCAGC |
| | WYQQKPGKAP | AGTCAGAGTATTAGTAGCTGGTTGG | IRQAPGKGLEWV | CTCTGGATTCACCTTTAGCAACTA |
| | NLLIYKASSLES | CCTGGTATCAGCAGAAACCAGGGA | STISSGGYNTNYA | TGCCATGACTTGGATCCGCCAGGC |
| | GVPSRFSGSGS | AAGCCCCTAACCTCCTGATCTATAA | DSVKGRFTISRDN | TCCAGGGAAGGGGCTGGAGTGGG |
| | GTEFTLTISGLQ | GGCGTCTAGTTTAGAAAGTGGGGTC | SKNTVYLQMSSL | TCTCAACTATTAGTAGTGGTGGTT |
| | PDDFATYYCQ | CCATCAAGGTTCAGCGGCAGTGGAT | RVEDTAVYYCVT | ATAATACAAACTACGCAGACTCCG |
| | QYNSYSLTFGQ | CTGGGACAGAATTCACTCTCACCAT | RRAGSYYAYWG | TGAAGGGCCGGTTCACCATTTCTA |
| | GTKVEIK | CAGCGGCCTGCAGCCTGATGATTTT | QGTLVTV | GAGACAATTCCAAGAACACGGTG |
| | (SEQ ID NO: 179; | GCAACTTATTACTGCCAACAGTATA | (SEQ ID NO: 180; | TATCTGCAAATGAGCAGCCTGAGA |
| | CDR1 SEQ ID | ATAGTTATTCATTGACGTTCGGCCA | CDR1 SEQ ID | GTCGAGGACACGGCCGTATATTAC |
| | NO: 309; CDR2 | AGGGACCAAGGTGGAAATCAAA | NO: 311; CDR2 | TGTGTGACTCGGAGAGCTGGGAGC |
| | SEQ ID NO: 276; | (SEQ ID NO: 138) | SEQ ID NO: 312; | TACTATGCCTACTGGGGCCAGGGA |
| | CDR3 SEQ ID | | CDR3 SEQ ID | ACCCTGGTCACCGTCT |
| | NO: 310) | | NO: 313) | (SEQ ID NO: 137) |
| KD11-1C2 | DIVMTQSPLSLP | GATATTGTGATGACTCAGTCTCCAC | QVQLVQSGAEVK | CAGGTGCAGCTGGTGCAGTCTGGG |
| | VTLGQPASISCR | TCTCCCTGCCCGTCACCCTTGGACA | KPGAPVKVSCKA | GCTGAGGTGAAGAAGCCTGGGGC |
| | SSQSLAYSDGN | GCCGGCCTCCATCTCCTGCAGGTCT | SGYTFTNYYMH | CCCAGTGAAGGTTTCCTGCAAGGC |
| | TYLNWFHQRP | AGCCAAAGCCTCGCATACAGTGATG | WVRQAPGQGLE | ATCTGGATACACCTTCACCAACTA |
| | GHSPRRLIYKVF | GAAACACCTACCTGAATTGGTTTCA | WLGLINAGSGRTR | CTATATGCACTGGGTGCGACAGGC |
| | NRDSGVPDRFS | CCAGAGGCCAGGCCACTCTCCAAGG | YAPKFQGRVTMT | CCCTGGACAAGGACTTGAGTGGCT |
| | GSGSGTDFTLKI | CGCCTAATTTATAAGGTTTTTAATCG | RDTSTSTLYIEVD | GGGACTAATCAACGCTGGTTCTGG |
| | SRVEAEDVGVY | GGACTCTGGGGTCCCAGACAGATTC | SLRSDDTAVYYC | TAGGACAAGGTACGCACCGAAGT |
| | YCMQGTHWPI | AGCGGCAGTGGGTCAGGCACTGATT | ARGSAHIEAGGS | TCCAGGGCAGAGTCACCATGACCA |
| | TFGQGTRLEIK | TCACACTGAAAATCAGCAGGGTGGA | AFDKWGQGTLV | GGGACACGTCCACGAGCACACTCT |
| | (SEQ ID NO: 181; | GGCTGAGGATGTTGGGGTTTATTAC | TV | ACATAGAGGTGGACAGCCTGAGA |
| | CDR1 SEQ ID | TGCATGCAAGGTACACACTGGCCGA | (SEQ ID NO: 182; | TCCGACGACACGGCCGTTTATTAC |
| | NO: 314; CDR2 | TCACCTTCGGCCAAGGGACACGACT | CDR1 SEQ ID | TGTGCGAGAGGATCCGCCCATATA |
| | SEQ ID NO: 315; | GGAGATTAAAC | NO: 317; CDR2 | GAAGCGGGTGGTTCCGCCTTTGAC |
| | CDR3 SEQ ID | (SEQ ID NO: 63) | SEQ ID NO: 318; | AAGTGGGGCCAGGGAACCCTGGT |
| | NO: 316) | | CDR3 SEQ ID | CACCGTCTC |
| | | | NO: 319) | (SEQ ID NO: 62) |
| KD11-1E9 | DIVMTQSPLSLP | GATATTGTGATGACTCAGTCTCCAC | QVQLVQSGAEVK | CAGGTGCAGCTGGTGCAGTCTGGG |
| | VILGQSASISCR | TGTCCCTGCCCGTCATCCTTGGACA | KPGAPVKVSCQA | GCTGAGGTGAAGAAGCCTGGGGC |
| | SSQSLIHSDGNI | GTCGGCCTCCATCTCCTGCAGGTCT | SGFIFINYYFHWV | CCCAGTGAAGGTTTCCTGCCAGGC |
| | FLNWFQQRPGQ | AGTCAAAGCCTCATACACAGTGATG | RQAPGQGLEWM | ATCTGGATTCATTTTCATCAATTAC |
| | SPRRLIYKVSNR | GGAACATCTTCTTGAATTGGTTTCA | GVINPTSGKSKLA | TATTTCCACTGGGTGCGGCAGGCC |
| | DSGVPDRFSGS | GCAGAGGCCAGGCCAATCTCCAAG | PTLENRVTMTSD | CCTGGACAAGGCCTGGAGTGGAT |
| | GSGTDFTLKISR | GCGCCTAATTTATAAGGTTTCTAAC | TSTRTLYIEVTSL | GGGAGTAATAAACCCCACTAGTG |
| | VEAEDVGVYY | CGGGACTCTGGGGTCCCAGACAGAT | TSDDTAIYYCAR | GTAAGTCAAAACTCGCGCCGACGT |
| | CMQGTHWPIT | TCAGCGGCAGTGGGTCAGGCACTGA | GSARAEAAGSAF | TGGAGAATCGAGTCACCATGACCA |
| | FGQGTRLEIK | TTTCACACTGAAAATCAGCAGGGTG | DHWGQGTLVTV | GCGACACGTCCACGAGAACACTTT |
| | (SEQ ID NO: 183; | GAGGCTGAGGATGTTGGCGTTTATT | (SEQ ID NO: 184; | ACATAGAGGTGACCAGCCTGACAT |
| | CDR1 SEQ ID | ACTGCATGCAAGGTACACACTGGCC | CDR1 SEQ ID | CTGACGACACGGCCATATATTACT |
| | NO: 320; CDR2 | GATCACCTTCGGCCAAGGGACACGA | NO: 322; CDR2 | GTGCGAGAGGGTCCGCCCGCGCA |
| | SEQ ID NO: 321; | CTGGAGATTAAAC | SEQ ID NO: 323; | GAAGCGGCTGGTTCCGCCTTTGAC |
| | CDR3 SEQ ID | (SEQ ID NO: 67) | CDR3 SEQ ID | CACTGGGGCCAGGGAACCCTGGTC |
| | NO: 316) | | NO: 324) | ACCGTCTC |
| | | | | (SEQ ID NO: 66) |
| KD11-2E10 | DIQMTQSPSSLS | GACATCCAGATGACCCAGTCTCCAT | QVQLQESGPGLV | CAGGTGCAGCTGCAGGAGTCGGG |
| | ASVGDRVTITC | CCTCCCTGTCTGCATCTGTAGGAGA | KPSETLSLTCTVS | CCCAGGACTGGTGAAGCCTTCGGA |
| | QASLDIKNYLN | CAGAGTCACCATCACTTGCCAGGCG | GASMNGYYWN | GACCCTGTCCCTCACCTGCACTGT |
| | WYQQKPGEAP | AGTCTGGACATTAAAAACTATCTAA | WIRQPPGKGLEW | CTCTGGTGCCTCCATGAATGGTTA |
| | KLLISDASTLET | ATTGGTATCAGCAGAAACCAGGGG | IGHIHYVGTTMKA | CTACTGGAACTGGATCCGGCAGCC |
| | GVPSRFSGRGS | AAGCCCCCAAGCTCCTGATCTC | TNDSPSLRSRVTI | CGACCCAGGGAAGGGACTGGAGTGGA |
| | GTHFTVTIRSLQ | TGCATCCACCTTGGAAACCGGGGTC | SVDTSRSQISLKL | TTGGACATATCCATTACGTTGGGA |
| | SEDVATYYCQ | CCATCAAGGTTCAGTGGAAGGGGGT | TSVTAADTAVYY | CTACCATGAAGGCCACCAACGAC |
| | QYGKLPFTFGP | CTGGGACACATTTCACTGTCACCAT | CARRRYHFWSD | AGCCCCTCCCTCAGGAGTCGAGTC |
| | GTKVDIK | CCGCAGCCTGCAGTCTGAAGATGTT | PGTFDIWGQGT | ACCATATCAGTGGACACGTCCAGG |
| | (SEQ ID NO: 185; | GCAACATATTACTGTCAACAATATG | MVTV | AGCCAGATCTCCCTGAAGCTGACG |
| | CDR1 SEQ ID | GTAAACTCCCATTCACTTTCGGCCCT | (SEQ ID NO: 186; | TCTGTGACCGCCGCGGACACGGCC |
| | NO: 325; CDR2 | GGGACCAAAGTGGATATCAAAC | CDR1 SEQ ID | GTGTATTACTGTGCGAGAAGGCGG |
| | SEQ ID NO: 326; | (SEQ ID NO: 77) | NO: 328; CDR2 | TACCATTTTTGGAGTGACCCCGGA |
| | CDR3 SEQ ID | | SEQ ID NO: 329; | ACGTTTGATATCTGGGGCCAAGGG |
| | NO: 327) | | CDR3 SEQ ID | ACAATGGTCACCGTCTC |
| | | | NO: 330) | (SEQ ID NO: 76) |
| KD7-2C1 | QLVLTQPSSLS | CAGCTTGTGCTGACTCAGCCGTCTT | EVQLLESGGGLV | GAGGTGCAGCTGTTGGAGTCTGGG |
| | ASPGASASLTC | CCCTGTCTGCATCTCCTGGAGCATC | QPGGSLRLSCAA | GGAGGCTTGGTACAGCCTGGGGG |
| | TLRSGINVGIYS | AGCCAGTCTCACCTGCACCTTACGC | SGFSFSTYAMSW | GTCCCTGAGACTCTCCTGTGCAGC |
| | IYWYQQKPGSP | AGTGGCATCAATGTTGGTATTTATA | VRQAPGKGLQW | CTCTGGATTCAGCTTTAGCACTTA |
| | PQYLLRFKSDS | GCATTTATTGGTACCAGCAGAAGCC | VSSISGNGYSSYY | TGCCATGAGCTGGGTCCGCCAGGC |
| | DKRQGSGVPSR | AGGGAGTCCTCCCCAGTATCTCCTG | ADSVKGRFHISR | TCCAGGAAAGGGGCTGCAGTGGG |
| | FSGSKDGSANA | AGGTTCAAATCAGACTCAGATAAAC | DNSKNTLSLEVN | TCTCAAGCATTAGTGGGAATGGTT |
| | AILVISGVQSED | GTCAGGGCTCTGGAGTCCCCAGCCG | SLGAEDTAVYFC | ACAGCTCATATTATGCAGACTCCG |
| | EADYYCMMW | CTTCTCTGGATCCAAAGATGGTTCG | AKGDGGSGWFG | TGAAGGGCCGCTTCCACATCTCCA |
| | HSSAWVFGGG | GCCAATGCAGCGATTTTAGTCATCT | FDYWGQGTLVT | GAGACAATTCCAAGAACACGCTGT |
| | TKLTVL | CTGGGGTCCAGTCTGAGGATGAGGC | V | CTCTGGAAGTGAACAGTCTGGGCG |
| | (SEQ ID NO: 187; | TGACTACTACTGTATGATGTGGCAC | (SEQ ID NO: 188; | CCGAGGACACGGCCGTCTATTTCT |
| | CDR1 SEQ ID | AGCAGCGCTTGGGTGTTCGGCGGAG | CDR1 SEQ ID | GTGCGAAAGGTGACGGTGGCAGT |
| | NO: 331; CDR2 | GGACCAAGCTGACCGTCCTAC | NO: 334; CDR2 | GGCTGGTTTGGTTTTGACTATTGG |
| | SEQ ID NO: 332; | (SEQ ID NO: 144) | SEQ ID NO: 335; | GGCCAGGGAACCCTGGTCACCGTC |
| | CDR3 SEQ ID | | CDR3 SEQ ID | T |
| | NO: 333) | | NO: 336) | (SEQ ID NO: 143) |
| KD9-1E10 | QSVLTQPPSAS | CAGTCTGTGCTGACGCAGCCACCCT | EVQLVQSGAEVK | GAGGTGCAGCTGGTGCAGTCTGGG |
| | GTPGQRVIISCS | CAGCGTCTGGGACCCCCGGGCAGAG | RPGASMKLSCKT | GCTGAGGTGAAACGGCCTGGGGC |
| | GGNSNIGSYTV | GGTCATTATTTCTTGTTCTGGGGGG | FGYIFTTYYIHWV | CTCAATGAAGCTTTCTTGCAAGAC |
| | QWYHQVPGTA | AACTCCAACATCGGAAGTTATACTG | RQAPGQGLEYLG | ATTTGGATACATCTTCACCACCTA |
| | PKLLIYRSNQRP | TCCAGTGGTACCATCAAGTCCCAGG | IINPAVGTTNFAQ | CTACATACACTGGGTGCGGCAGGC |
| | SGVPDRFSGSK | AACGGCCCCCAAACTCCTCATCTAT | KFKGRLSMTRDT | CCCTGGACAAGGTCTTGAATACTT |
| | SGTSASLAISGL | AGGAGTAATCAGCGGCCCTCAGGG | STSTVYMELSSPT | GGGAATAATCAACCCTGCTGTTGG |
| | QSEDEADYYCS | GTCCCTGACCGATTCTCTGGCTCCA | SDDSAVYYCARE | TACCACAAACTTCGCACAGAAGTT |
| | AWDDSLNAYV | AGTCTGGCACCTCAGCCTCCCTGGC | LHATHFFDFWG | CAAGGGCAGGCTCAGCATGACCA |
| | FGTGTRVAVL | CATCAGTGGGCTCCAGTCTGAAGAT | QGTLVTV | GGGACACGTCCACGAGCACAGTCT |
| | (SEQ ID NO: 189; | GAGGCTGATTATTACTGTTCAGCAT | (SEQ ID NO: 190; | ACATGGAACTGAGCAGCCCGACA |
| | CDR1 SEQ ID | GGGATGACAGCCTGAATGCTTATGT | CDR1 SEQ ID | TCTGACGACTCGGCCGTCTATTAC |
| | NO: 337; CDR2 | CTTCGGAACTGGGACCAGGGTCGCC | NO: 340; CDR2 | TGTGCGAGGGAACTACACGCTACA |
| | SEQ ID NO: 338; | GTCCTAG | SEQ ID NO: 341; | CACTTCTTCGACTTCTGGGGCCAG |
| | CDR3 SEQ ID | (SEQ ID NO: 160) | CDR3 SEQ ID | GGAACCCTGGTCACCGTCT |
| | NO: 339) | | NO :342) | (SEQ ID NO: 159) |

TABLE 1B
Monoclonal Antibody Nucleotide Sequences. Sequence names
ending in “L” of “K” indicate a nucleotide
sequence that encodes a light chain variable domain
and the remainder of the sequence names indicate a nucleotide
sequence that encodes a heavy chain variable domain.

| KD Patient# | Sequence Name | (SEQ ID NO:) |
|---|---|---|
| KD1 | 1A4 | 1 |
| KD1 | 1A5 | 2 |
| KD1 | 1A7 | 3 |
| KD1 | 1A9 | 4 |
| KD1 | 1B2 | 5 |
| KD1 | 1B8 | 6 |
| KD1 | 1C4 | 7 |
| KD1 | 1C5 | 8 |
| KD1 | 1C6 | 9 |
| KD1 | 1C6K | 10 |
| KD1 | 1F1 | 11 |
| KD1 | 1F1K | 12 |
| KD1 | 1F3 | 13 |
| KD1 | 1F3K | 14 |
| KD1 | 1F6H | 15 |
| KD1 | 1F6L | 16 |
| KD1 | 1F7 | 17 |
| KD1 | 1G8 | 18 |
| KD1 | 1H10 | 19 |
| KD1 | 1H12 | 20 |
| KD1 | 2A10 | 21 |
| KD1 | 2A3 | 22 |
| KD1 | 2A5 | 23 |
| KD1 | 2A5L | 24 |
| KD1 | 2A8 | 25 |
| KD1 | 2A8K | 26 |
| KD1 | 2B11 | 27 |
| KD1 | 2B9 | 28 |
| KD1 | 2C2 | 29 |
| KD1 | 2C2K | 30 |
| KD1 | 2F3 | 31 |
| KD1 | 2G10 | 32 |
| KD1 | 2G11 | 33 |
| KD1 | 2G11K | 34 |
| KD1 | 2G6 | 35 |
| KD1 | 2H10 | 36 |
| KD1 | 2H10K | 37 |
| KD10 | 1A10 | 38 |
| KD10 | 1A10K | 39 |
| KD10 | 1B1 | 40 |
| KD10 | 1B1K | 41 |
| KD10 | 1B8 | 42 |
| KD10 | 1B8K | 43 |
| KD10 | 1E9 | 44 |
| KD10 | 1E9L | 45 |
| KD10 | 1F11 | 46 |
| KD10 | 1F11L | 47 |
| KD10 | 1G12 | 48 |
| KD10 | 1G12L | 49 |
| KD10 | 2A1 | 50 |
| KD10 | 2A1L | 51 |
| KD10 | 2E5 | 52 |
| KD10 | 2G12 | 53 |
| KD11 | 1A12 | 54 |
| KD11 | 1A12L | 55 |
| KD11 | 1B12 | 56 |
| KD11 | 1B12K | 57 |
| KD11 | 1B7 | 58 |
| KD11 | 1B7L | 59 |
| KD11 | 1C1 | 60 |
| KD11 | 1C1K | 61 |
| KD11 | 1C2 | 62 |
| KD11 | 1C2K | 63 |
| KD11 | 1D6 | 64 |
| KD11 | 1D6L | 65 |
| KD11 | 1E9 | 66 |
| KD11 | 1E9K | 67 |
| KD11 | 1F6 | 68 |
| KD11 | 1F6K | 69 |
| KD11 | 2A12 | 70 |
| KD11 | 2A12K | 71 |
| KD11 | 2D10 | 72 |
| KD11 | 2D10K | 73 |
| KD11 | 2D5 | 74 |
| KD11 | 2D5K | 75 |
| KD11 | 2E10 | 76 |
| KD11 | 2E10K | 77 |
| KD11 | 2E7 | 78 |
| KD11 | 2E7K | 79 |
| KD11 | 2F12 | 80 |
| KD11 | 2F12K | 81 |
| KD11 | 2G1 | 82 |
| KD11 | 2G1K | 83 |
| KD11 | 2H6 | 84 |
| KD11 | 2H6L | 85 |
| KD2 | 1A7 | 86 |
| KD2 | 1A7L | 87 |
| KD2 | 2B6 | 88 |
| KD2 | 2B6L | 89 |
| KD2 | 2C7 | 90 |
| KD2 | 2C7L | 91 |
| KD2 | 2F8 | 92 |
| KD2 | 2F8L | 93 |
| KD3 | 1G8 | 94 |
| KD3 | 1G8L | 95 |
| KD3 | 1G9 | 96 |
| KD3 | 1G9L | 97 |
| KD4 | 1F5 | 98 |
| KD4 | 1F5L | 99 |
| KD4 | 2C5 | 100 |
| KD4 | 2C5K | 101 |
| KD4 | 2H4 | 102 |
| KD4 | 2H4L | 103 |
| KD5 | 1C10 | 104 |
| KD5 | 1C10K | 105 |
| KD5 | 1E2 | 106 |
| KD5 | 1E2K | 107 |
| KD5 | 1E2L | 108 |
| KD5 | 2C1 | 109 |
| KD5 | 2C10 | 110 |
| KD5 | 2C10L | 111 |
| KD5 | 2C1K | 112 |
| KD5 | 2C6 | 113 |
| KD5 | 2C6K | 114 |
| KD5 | 2D3 | 115 |
| KD5 | 2D3K | 116 |
| KD5 | 2F6 | 117 |
| KD5 | 2F6K | 118 |
| KD6 | 1A10 | 119 |
| KD6 | 1A10L | 120 |
| KD6 | 1A2 | 121 |
| KD6 | 1A2L | 122 |
| KD6 | 1D1 | 123 |
| KD6 | 1D1L | 124 |
| KD6 | 2A12 | 125 |
| KD6 | 2A12L | 126 |
| KD6 | 2B2 | 127 |
| KD6 | 2B2K | 128 |
| KD6 | 2F4 | 129 |
| KD6 | 2F4L | 130 |
| KD6 | 2G3 | 131 |
| KD6 | 2G3K | 132 |
| KD6 | 2H3 | 133 |
| KD6 | 2H3K | 134 |
| KD7 | 1A12 | 135 |
| KD7 | 1A12K | 136 |
| KD7 | 1D3 | 137 |
| KD7 | 1D3K | 138 |
| KD7 | 1D9 | 139 |
| KD7 | 1D9L | 140 |
| KD7 | 2A9 | 141 |
| KD7 | 2A9K | 142 |
| KD7 | 2C1 | 143 |
| KD7 | 2C1L | 144 |
| KD7 | 2F9 | 145 |
| KD7 | 2F9K | 146 |
| KD8 | 1B10 | 147 |
| KD8 | 1B10K | 148 |
| KD8 | 1D4 | 149 |
| KD8 | 1D4K | 150 |
| KD8 | 2F3 | 151 |
| KD8 | 2F3L | 152 |
| KD9 | 1A10 | 153 |
| KD9 | 1A10K | 154 |
| KD9 | 1B5 | 155 |
| KD9 | 1B5L | 156 |
| KD9 | 1D2 | 157 |
| KD9 | 1D2L | 158 |
| KD9 | 1E10 | 159 |
| KD9 | 1E10L | 160 |
| KD9 | 2C5 | 161 |
| KD9 | 2C5L | 162 |
| KD9 | 2C8 | 163 |
| KD9 | 2C8L | 164 |

TABLE 1C
Strong Binding Monoclonal Antibodies:

| KDPt # | Sequence Name | SEQ ID NO: |
|---|---|---|
| KD1 | 2G11 | 33 |
| KD1 | 2G11K | 34 |
| KD11 | 1C2 | 62 |
| KD11 | 1C2K | 63 |
| KD11 | 1E9 | 66 |
| KD11 | 1E9K | 67 |
| KD11 | 2E10 | 76 |
| KD11 | 2E10K | 77 |
| KD4 | 2H4 | 102 |
| KD4 | 2H4L | 103 |
| KD6 | 1A10 | 119 |
| KD6 | 1A10L | 120 |
| KD6 | 2B2 | 127 |
| KD6 | 2B2K | 128 |
| KD7 | 1D3 | 137 |
| KD7 | 1D3K | 138 |
| KD7 | 2C1 | 143 |
| KD7 | 2C1L | 144 |
| KD9 | 1E10 | 159 |
| KD9 | 1E10L | 160 |

TABLE 1D
Weak Binding Monoclonal Antibodies:

| KDPt # | Sequence Name | SEQ ID NO: |
|---|---|---|
| KD1 | 2H10 | 36 |
| KD1 | 2H10K | 37 |
| KD10 | 1A10 | 38 |
| KD10 | 1A10K | 39 |
| KD10 | 2A1 | 50 |
| KD10 | 2A1L | 51 |
| KD11 | 1B12 | 56 |
| KD11 | 1B12K | 57 |
| KD11 | 1C1 | 60 |
| KD11 | 1C1K | 61 |
| KD11 | 1D6 | 64 |
| KD11 | 1D6L | 65 |
| KD11 | 1F6 | 68 |
| KD11 | 1F6K | 69 |
| KD11 | 2E7 | 78 |
| KD11 | 2E7K | 79 |
| KD11 | 2F12 | 80 |
| KD11 | 2F12K | 81 |
| KD11 | 2G1 | 82 |
| KD11 | 2G1K | 83 |
| KD11 | 2H6 | 84 |
| KD11 | 2H6L | 85 |
| KD5 | 2C1 | 109 |
| KD5 | 2C1K | 112 |
| KD6 | 1A2 | 121 |
| KD6 | 1A2L | 122 |
| KD6 | 2A12 | 125 |
| KD6 | 2A12L | 126 |
| KD6 | 2H3 | 133 |
| KD6 | 2H3K | 134 |
| KD7 | 2A9 | 141 |
| KD7 | 2A9K | 142 |
| KD8 | 1B10 | 147 |
| KD8 | 1B10K | 148 |
| KD8 | 1D4 | 149 |
| KD8 | 1D4K | 150 |
| KD9 | 1A10 | 153 |
| KD9 | 1A10K | 154 |
| KD9 | 1B5 | 155 |
| KD9 | 1B5L | 156 |
| KD9 | 1D2 | 157 |
| KD9 | 1D2L | 158 |
| KD9 | 2C5 | 161 |
| KD9 | 2C5L | 162 |

In some aspects, a monoclonal antibody described herein includes a light chain variable region selected from the group consisting of SEQ ID NOs:165, 167, 169, 171, 173, 175, 177, 179, 181, 183, 185, 187, and 189, or a sequence substantially identical thereto, and a heavy chain comprising a rabbit heavy chain constant domain and a heavy chain variable region selected from the group consisting of SEQ ID NOs:166, 168, 170, 172, 174, 176, 178, 180, 182, 184, 186, 188, and 190, or a sequence substantially identical thereto.

In some aspects, an antibody as described herein includes the light chain variable region of SEQ ID NO:165 and the heavy chain variable region of SEQ ID NO:166, the light chain variable region of SEQ ID NO:167 and the heavy chain variable region of SEQ ID NO:168, the light chain variable region of SEQ ID NO:169 and the heavy chain variable region of SEQ ID NO:170, the light chain variable region of SEQ ID NO:171 and the heavy chain variable region of SEQ ID NO:172, the light chain variable region of SEQ ID NO: 173 and the heavy chain variable region of SEQ ID NO: 174, the light chain variable region of SEQ ID NO: 175 and the heavy chain variable region of SEQ ID NO: 176, the light chain variable region of SEQ ID NO:177 and the heavy chain variable region of SEQ ID NO: 178, the light chain variable region of SEQ ID NO:179 and the heavy chain variable region of SEQ ID NO:180, the light chain variable region of SEQ ID NO:181 and the heavy chain variable region of SEQ ID NO:182, the light chain variable region of SEQ ID NO:183 and the heavy chain variable region of SEQ ID NO:184, the light chain variable region of SEQ ID NO:185 and the heavy chain variable region of SEQ ID NO:186, the light chain variable region of SEQ ID NO:187 and the heavy chain variable region of SEQ ID NO:188, or the light chain variable region of SEQ ID NO:189 and the heavy chain variable region of SEQ ID NO: 190.

In some aspects, provided herein is a nucleic acid or polynucleotide encoding an antibody described herein.

Protein and nucleic acid sequence identities are evaluated using the Basic Local Alignment Search Tool (“BLAST”) which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 85% sequence identity to the SEQ ID. Alternatively, percent identity can be any integer from 85% to 100%. More preferred embodiments include at least: 85%, 86%, 87% 88%, 89%, 90%, 91% 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning, and the like.

“Substantial identity” of amino acid sequences for purposes of this invention normally means polypeptide sequence identity of at least 85%. Preferred percent identity of polypeptides can be any integer from 85% to 100%. More preferred embodiments include at least 85%, 86%, 87%, 88%, 89%, 90%, 91%, 92%, 93%, 94%, 95%, 96%, 97%, 98%, or 99%.

In some embodiments, the isolated antibody or fragment thereof is directly or indirectly linked to a tag or agent. In some embodiments, the antibody or fragment thereof is conjugated to the tag or agent. In other embodiments, the tag or agent is a polypeptide, wherein the polypeptide is translated concurrently with the antibody polypeptide sequence.

The term “tag” or “agent” as used herein includes any useful moiety that allows for the purification, identification, detection, diagnosing, imaging, or therapeutic use of the antibody of the present invention. The terms tag or agent includes epitope tags, detection markers and/or imaging moieties, including, for example, enzymatic markers, fluorescence markers, radioactive markers, among others. Additionally, the term tag or agent includes therapeutic agents, small molecules, and drugs, among others. The term tag or agent also includes diagnostic agents. In some embodiments, the tag is a biotinylated tag.

### Methods of Detection, Imaging and Diagnosing

The antibodies disclosed herein can be used for methods of assaying, detecting, imaging, and diagnosing Kawasaki Disease (KD) or the presence of intracytoplasmic inclusion bodies (ICI).

One embodiment provides a method of detecting ICI in a sample, wherein the method comprises contacting the sample with an antibody described herein, and detecting the binding of the antibody in the sample. An increase in binding of the antibody to the sample as compared to binding of the antibody to a negative control sample detects ICI within the sample.

The term “contacting” or “exposing,” as used herein refers to bringing a disclosed antibody and a cell, a target receptor, a biological sample, or other biological entity, together in such a manner that the antibody can detect and/or affect the activity of the target, either directly; i.e., by interacting with the target itself, or indirectly; i.e., by interacting with another molecule, co-factor, factor, or protein that is attached to said target.

Another embodiment provides a method of diagnosing KD in a subject by contacting a sample from the subject with the antibody disclosed herein and detecting the binding of the antibody to the sample. In some embodiments, the sample from the subject is compared to a control sample. An increased binding of the antibody in the sample as compared to the control sample confirms the diagnosis of KD. A negative signal, e.g. no binding of the antibody, signals that KD is not present.

Suitable methods of detection are known in the art and include, but are not limited to, for example, ELISA, Western blot, immunostaining, immunoprecipitation, flow cytometry, sensor chips, magnetic beads, and the like.

As used herein “sample” or “biological sample” refers to a sample taken from a subject, such as but not limited to, a blood or tissue sample.

The present invention has been described in terms of one or more preferred embodiments, and it should be appreciated that many equivalents, alternatives, variations, and modifications, aside from those expressly stated, are possible and within the scope of the invention.

### Example 1

The embodiment described here demonstrates the development of monoclonal antibodies based on plasmablasts isolated from subjects diagnosed with KD. The embodiment described in this example also demonstrates the binding of the monoclonal antibodies to hepacivirus C NS4A.

Traditional approaches of cultivating an infectious agent from patient tissues, as well as molecular biology approaches such as deep sequencing of KD tissues (7), have been unrevealing, indicating that alternative approaches to solve this problem are needed. Analysis of peripheral blood plasmablasts is emerging as a powerful tool in studies of pathogenesis, diagnosis, and therapeutic discovery in infectious diseases (8-16), vaccine science (16-22), and autoimmune disease (23, 24). Multiple studies have shown that >75% of peripheral blood plasmablasts express antibodies specific to antigenic targets of an ongoing immune response (25-28). Identification of specific KD antigens would facilitate diagnostic test development and improved therapies.

We discovered an oligoclonal IgA response in KD arterial tissues (29), and made “first generation” synthetic antibodies comprised of oligoclonal alpha heavy chains paired with random light chains (30, 31) as tools to identify the target antigen(s). These antibodies identified ICI in KD but not in infant control ciliated bronchial epithelium by immunohistochemistry (30-32). However, the antibodies did not yield specific antigen by Western blot, immunoprecipitation, or screening of poly A-selected KD arterial, spleen, or liver expression cDNA libraries.

We hypothesized that our original KD synthetic antibodies were not optimal for specific antigen identification because they did not include in vivo cognate light and heavy chains. We thought it likely that antigen-specific antibody-producing cells were present in the blood of KD children, since these cells were present in the coronary arteries (29, 30). Therefore, we isolated peripheral blood plasmablasts from children with KD 1-3 weeks after fever onset using single cell sorting by flow cytometry, analyzed their cognate light and heavy chain sequences, and prepared “second generation” synthetic antibodies from oligoclonal plasmablasts. Our goal was to use the KD-specific B cell response to identify antigens important in KD pathogenesis.

### Results

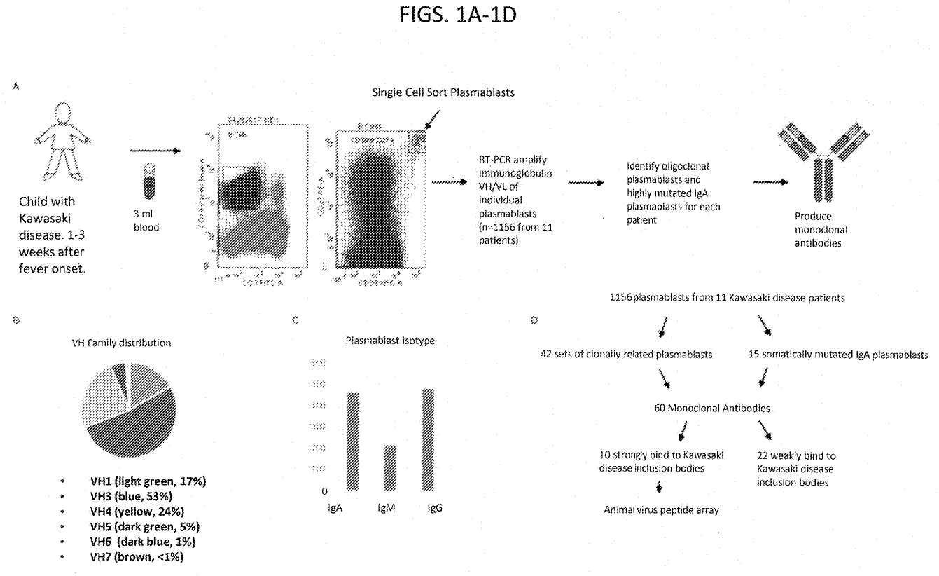

Plasmablast isolation from selected KD patients˜KD is a clinical diagnosis, but can be considered confirmed in a young child with a prolonged febrile illness who develops coronary artery aneurysms (3). To be certain that we included children with definite KD in this study, we preferentially enrolled those with coronary artery aneurysms at diagnosis and those who appeared to be at high risk for such complications (3). By convention, the first day of illness in KD is defined as the first day of fever (3). Plasmablasts from 11 KD patients (FIG. 1A, Table 2A) on day 8-24 following fever onset were single cell sorted into two 96-well plates per patient. The sample timing was based on prior studies by others indicating antigen-specific plasmablast responses peak at day 6-7 but may persist for up to 2-3 weeks following acute infection (32). Heavy chain sequences were identified in 1156 plasmablasts derived from 11 patients. The remaining wells of the plates either did not contain a plasmablast or the plasmablast heavy chain sequences were out of frame or had a stop codon. Most of the plasmablasts belonged to the VH3 family (FIG. 1B); 462 were IgA, 482 were IgG, and 212 were IgM (FIG. 1C). The number of functional heavy chain sequences obtained per patient varied, likely because of variable time from sample collection to plasmablast isolation (range, 2 hours to 24 hours) and other technical factors. In general, a greater number of plasmablast sequences were obtained when blood was processed immediately after the sample was obtained. Plasmablasts constituted <0.1 to 1.8% of total circulating B cells in KD patients in this study.

TABLE 2A
Clinical and plasmablast data for Kawasaki disease patients in this study.

| | | | | | | Day of | | | |
|---|---|---|---|---|---|---|---|---|---|
| | | | | | Day of | illness | | | Total # |
| | | | | Race/ | illness | IVIG | Incomplete | Zmax | Plasmablasts |
| Patient | Age | | Gender | Ethnicity | of sample | given | KD | RCA/LAD | (isotype) |
| KD1 | 4 | yr | F | Hispanic | 17 | 13 | No | RCA 4.7 | 119 (49A, |
| | | | | | | | | | 28G, 42M) |
| KD2 | 4 | yr | F | Caucasian | 24 | 8 | No | NL | 115 (64A, |
| | | | | | | | | | 22G, 29M) |
| KD3 | 5 | yr | M | Black | 20 | 19 | Yes | NL | 67 (33A, |
| | | | | | | | | | 31G, 3M) |
| KD4 | 3 | mo | M | Hispanic | 8 | 6 | Yes | RCA 3.0, | 153 (102G, |
| | | | | | | | | LAD 3.5 | 30A, 21M) |
| KD5 | 11 | mo | F | ½ | 14 | 10 | Yes | NL | 115 (63G, |
| | | | | Caucasian, | | | | | 39A, 13M) |
| | | | | ½ Asian | | | | | |
| | | | | (Vietnamese) | | | | | |
| KD6 | 4 | yr | F | Black | 13 | 5 and 8 | No | RCA 3.0 | 80 (43A, |
| | | | | | | | | | 23G, 14M) |
| KD7 | 17 | mo | M | Caucasian | 13 | 9 | No | RCA 9.8, | 121 (56A, |
| | | | | | | | | LAD 5.7 | 50G, 15M) |
| KD8 | 17 | mo | M | Caucasian | 14 | 12 | Yes | LAD 3.5 | 85 (41A, |
| | | | | | | | | | 34G, 10M) |
| KD9 | 3 | yr | M | Asian | 9 | 6 | No | NL | 81 (30A, |
| | | | | (Indian) | | | | | 38G, 13M) |
| KD10 | 5 | yr | F | Asian (½ | 11 | 7 | No | RCA 5.4, | 88 (41A, |
| | | | | Japanese, | | | | LAD 7.4, | 41G, 6M) |
| | | | | ½ Korean) | | | | distal large | |
| | | | | | | | | R, L CAA | |

Genetic characterization of KD plasmablasts reveals an oligoclonal response—Forty-two sets of clonally related plasmablasts were identified in ten patients (FIG. 1D). One patient (KD7) among those studied did not have clonally related plasmablasts, but did have IgA plasmablasts with many mutations from germline (Table 2B). More than one isotype was present in 12/42 (29%) of the clonally related plasmablast sets from these 10 patients (Table 2B). KD plasmablasts were otherwise of varying genetic composition, and the clonally related heavy chain CDR3 sequences differed among patients. This result was expected based on published data showing that the VH nucleotide repertoire is highly private (33, 34). Four of the KD patients had VH3-21 clonally related plasmablasts (with differing CDR3 sequence), but no other VH family preferences were apparent in this study (Table 2B). Clonally related sets of plasmablasts were selected for antibody production, and two versions of the antibodies were prepared when the set contained members with CDR3 sequence differences. In a subset of patients, we also prepared antibodies from selected IgA plasmablasts with many somatic mutations (FIG. 1D, Table 2B). VDJ and VJ sequences of these plasmablasts and for healthy adult volunteer plasmablasts E3 and E4 are available as GenBank accession numbers MK416266-MK417513.

TABLE 2B
Genetic characteristics of KD plasmablasts in this study

| | | | | | CDR3 | Number of | | | CDR3 | Number of | Number of |
|---|---|---|---|---|---|---|---|---|---|---|---|
| Patient # | | Heavy chain | | | length | mutations | Light chain | | length | mutations | plasmablasts |
| (total PB) | mAb | IGVH | IGVD | IGVJ | (aa) | (aa) | IGV | IGJ | (aa) | (aa) | (G, A, M) |
| KD1 (119) | 1F3 | IGHV3- | IGHD3- | IGHJ4*02 | 10 | 5 | IGKV2- | IGKJ4*01 | 8 | 2 | 12 (1, 10, 1) |
| | | 7*01 | 10*01 | | | | 29*02 | | | | |
| | 2H10 | IGHV3- | IGHD1- | IGHJ4*02 | 10 | 6 | IGKV2- | IGKJ4*01 | 8 | 2 | |
| | | 7*01 | 26*01 | | | | 29*02 | | | | |
| | 2A8 | IGHV3- | IGHD3- | IGHJ5*02 | 10 | 10 | IGKV2- | IGKJ1*01 | 9 | 7 | 1 (0, 1, 0) |
| | | 7*01 | 16*01 | | | | 30*02 | | | | |
| | 1F1 | IGHV3- | IGHD7- | IGHJ4*02 | 16 | 8 | IGKV3- | IGKJ1*01 | 10 | 3 | 2 (0, 2, 0) |
| | | 21*01 | 27*01 | | | | 15*01 | | | | |
| | 1C6 | IGHV4- | IGHD1- | IGHJ5*02 | 13 | 10 | IGKV4- | IGKJ1*01 | 10 | 2 | 2 (1, 1, 0) |
| | | 39-*01 | 1*01 | | | | 1*01 | | | | |
| | 2G11 | IGHV3- | IGHD5- | IGHJ4*02 | 15 | 10 | IGKV1- | IGKJ3*01 | 9 | 10 | 2 (0, 1, 1) |
| | | 15*01 | 24*01 | | | | 39*01 | | | | |
| KD2 (115) | 2B6 | IGHV3- | IGHD4- | IGHJ4*02 | 12 | 18 | IGLV3- | IGLJ2*01 | 10 | 9 | 2 (0, 1, 1) |
| | | 15*05 | 23*01 | | | | 10*01 | | | | |
| | 2F8 | IGHV3- | IGHD3- | IGHJ4*02 | 15 | 12 | IGLV3- | IGLJ3*02 | 11 | 9 | 1 (0, 1, 0) |
| | | 73*02 | 16*01 | | | | 25*02 | | | | |
| | 1A7 | IGHV3- | IGHD3- | IGHJ4*02 | 15 | 8 | IGLV3- | IGLJ3*02 | 12 | 13 | 1 (0, 1, 0) |
| | | 73*02 | 22*01 | | | | 25*03 | | | | |
| | 2C7 | IGHV6- | IGHD5- | IGHJ4*02 | 13 | 6 | IGLV1- | IGLJ1*01 | 11 | 8 | 3 (1, 2, 0) |
| | | 1*01 | 12*01 | | | | 51*02 | | | | |
| KD3 (67) | 1G8 | IGHV5- | IGHD3- | IGHJ4*02 | 10 | 6 | IGLV2- | IGLJ3*02 | 10 | 13 | 2 (0, 2, 0) |
| | | 10-1*03 | 10*01 | | | | 14*01 | | | | |
| | 1G9 | IGHV1- | IGHD3- | IGHJ4*02 | 9 | 10 | IGLV8- | IGLJ2*01 | 10 | 10 | 2 (2, 0, 0) |
| | | 18*04 | 9*01 | | | | 61*01 | | | | |
| KD4 (153) | 2C5 | IGHV1- | IGHD2- | IGHJ4*02 | 14 | 1 | IGKV3- | IGKJ2*01 | 10 | 0 | 2 (2, 0, 0) |
| | | 3*01 | 2*01 | | | | 20*01 | | | | |
| | 2H4 | IGHV3- | IGHD7- | IGHJ2*01 | 14 | 4 | IGLV2- | IGLJ2*01 | 10 | 0 | 2 (1, 0, 1) |
| | | 74*01 | 27*01 | | | | 11*01 | | | | |
| | 1F5 | IGHV5- | IGHD4- | IGHJ6*03 | 19 | 0 | IGLV2- | IGLJ3*02 | 10 | 0 | 2 (2, 0, 0) |
| | | 51*01 | 11*01 | | | | 14*01 | | | | |
| KD5 (115) | 1E2 | IGHV5- | IGHD6- | IGHJ4*02 | 13 | 4 | IGLV4- | IGLJ3*02 | 9 | 0 | 2 (2, 0, 0) |
| | | 51*03 | 19*01 | | | | 60*03 | | | | |
| | 1C10 | IGHV5- | IGHD3- | IGHJ3*02 | 16 | 5 | IGKV3- | IGKJ5*01 | 9 | 3 | 2 (2, 0, 0) |
| | | 51*01 | 9*01 | | | | 20*01 | | | | |
| | 2C1 | IGHV4- | IGHD6- | IGHJ4*02 | 16 | 1 | IGK3- | IGKJ2*01 | 11 | 0 | 3 (2, 1, 0) |
| | | 61*02 | 13*01 | | | | 20*01 | | | | |
| | 2C6 | IGHV3- | IGHD3- | IGHJ4*02 | 13 | 7 | IGKV1- | IGKJ1*01 | 9 | 1 | 2 (0, 1, 1) |
| | | 23*01 | 22*01 | | | | 5*03 | | | | |
| | 2C10 | IGHV3- | IGHD3- | IGHJ4*02 | 15 | 15 | IGLV4- | IGLJ3*02 | 10 | 4 | 2 (2, 0, 0) |
| | | 23*01 | 10*01 | | | | 69*01 | | | | |
| | 2D3 | IGHV4- | IGHD3- | IGHJ5*02 | 15 | 7 | IGKV4- | IGKJ1*01 | 9 | 3 | 2 (0, 2, 0) |
| | | 61*02 | 22*01 | | | | 1*01 | | | | |
| | 2F6 | IGHV1- | IGHD3- | IGHJ4*02 | 13 | 7 | IGKV1- | IGKJ2*04 | 8 | 4 | 2 (0, 1, 1) |
| | | 8*01 | 10*01 | | | | 39*01 | | | | |
| KD6 (80) | 1A2 | IGHV1- | IGHD3- | IGHJ3*01 | 13 | 17 | IGLV7- | IGLJ2*01 | 10 | 12 | 3 (0, 3, 0) |
| | | 45*02 | 9*01 | | | | 46*01 | | | | |
| | 2A12 | IGHV1- | IGHD2- | IGHJ3*01 | 13 | 17 | SAME | SAME | 10 | 11 | |
| | | 45*02 | 8*01 | | | | | | | | |
| | 1A10 | IGHV3- | IGHD3- | IGHJ4*02 | 20 | 11 | IGLV1- | IGLJ3*02 | 10 | 11 | 2 (0, 2, 0) |
| | | 33*03 | 22*01 | | | | 44*01 | | | | |
| | 2F4 | IGHV1- | IGHD2- | IGHJ4*02 | 17 | 9 | IGLV2- | IGLJ2*01 | 10 | 8 | 2 (0, 2, 0) |
| | | 45*02 | 2*01 | | | | 11*01 | | | | |
| | 2G3 | IGHV3- | IGHD5- | IGHJ2*01 | 18 | 11 | IGKV3- | IGKJ4*01 | 8 | 11 | 2 (2, 0, 0) |
| | | 21*01 | 12*01 | | | | 20*01 | | | | |
| | 2H3 | IGHV3- | IGHD3- | IGHJ3*01 | 19 | 9 | IGKV3- | IGKJ4*01 | 8 | 5 | 2 (1, 0, 1) |
| | | 15*01 | 10*01 | | | | 15*01 | | | | |
| | 2B2 | IGHV3- | IGHD3- | IGHJ3*01 | 12 | 15 | IGKV1- | IGKJ1*01 | 10 | 9 | 2 (0, 2, 0) |
| | | 33*01 | 3*01 | | | | 5*03 | | | | |
| KD7 (121) | 1A12 | IGHV3- | IGHD4- | IGHJ4*02 | 9 | 9 | IGKV1- | IGKJ1*01 | 9 | 4 | 1 (0, 1, 0) |
| | | 7*03 | 17*01 | | | | 5*03 | | | | |
| | 1D3 | IGHV3- | IGHD1- | IGHJ4*02 | 11 | 15 | IGKV1- | IGKJ1*01 | 9 | 3 | 1 (0, 1, 0) |
| | | 23/ | 26*01 | | | | 5*03 | | | | |
| | | 23D*01 | | | | | | | | | |
| | 1D9 | IGHV5- | IGHD6- | IGHJ4*02 | 14 | 10 | IGLV1- | IGLJ1*01 | 11 | 7 | 1 (0, 1, 0) |
| | | 10-1*03 | 25*01 | | | | 44*01 | | | | |
| | 2A9 | IGHV5- | IGHD5- | IGHJ4*02 | 13 | 20 | IGKV1- | IGKJ1*01 | 9 | 11 | 1 (0, 1, 0) |
| | | 10-1*03 | 24*01 | | | | 5*03 | | | | |
| | 2C1 | IGHV3- | IGHD6- | IGHJ4*02 | 14 | 13 | IGLV5- | IGLJ3*02 | 9 | 10 | 1 (0, 1, 0) |
| | | 23/ | 19*01 | | | | 45*02 | | | | |
| | | 23D*01 | | | | | | | | | |
| | 2F9 | IGHV3- | IGHD2- | IGHJ4*02 | 12 | 17 | IGKV2- | IGKJ2*01 | 9 | 5 | 1 (0, 1, 0) |
| | | 23/ | 21*01 | | | | 24*01 | | | | |
| | | 23D*01 | | | | | | | | | |
| KD8 (85) | 1B10 | IGHV3- | IGHD3- | IGHJ3*02 | 12 | 3 | IGKV1- | IGKJ4*01 | 9 | 6 | 2 (0, 2, 0) |
| | | 72*01 | 16*01 | | | | 6*01 | | | | |
| | 1D4 | IGHV3- | IGHD3- | IGHJ3*02 | 12 | 1 | IGKV1- | IGKJ4*01 | 9 | 4 | |
| | | 72*01 | 9*01 | | | | 6*01 | | | | |
| KD9 (81) | 1D2 | IGHV1- | IGHD5- | IGHJ4*02 | 11 | 5 | IGLV7- | IGLJ3*02 | 9 | 8 | 2 (1, 1, 0) |
| | | 46*01 | 18*01 | | | | 43*01 | | | | |
| | 2C8 | IGHV1- | IGHD2- | IGHJ4*02 | 11 | 5 | IGLV7- | IGLJ3*02 | 9 | 5 | |
| | | 46*01 | 15*01 | | | | 43*01 | | | | |
| | 1E10 | IGHV1- | IGHD4- | IGHJ4*02 | 12 | 22 | IGLV1- | IGLJ1*01 | 11 | 10 | 1 (0, 1, 0) |
| | | 46*01 | 11*01 | | | | 44*01 | | | | |
| | 2C5 | IGHV4- | IGHD3- | IGHJ6*02 | 14 | 4 | IGLV2- | IGLJ1*01 | 10 | 7 | 2 (0, 0, 2) |
| | | 59*08 | 10*01 | | | | 23*01 | | | | |
| | 1B5 | IGHV4- | IGHD3- | IGHJ6*02 | 14 | 2 | IGLV2- | IGLJ1*01 | 10 | 6 | |
| | | 59*08 | 10*01 | | | | 23*01 | | | | |
| | 1A10 | IGHV3- | IGHD3- | IGHJ4*02 | 16 | 20 | IGLKV3- | IGKJ2*01 | 9 | 13 | 1 (0, 1, 0) |
| | | 30*02 | 10*01 | | | | 11*01 | | | | |
| KD10 (88) | 1F11# | IGHV3- | IGHD3- | IGHJ4*02 | 12 | 14 | IGLV2- | IGLJ1*01 | 11 | 6 | 5 (5, 0, 0) |
| | | 7*03 | 10*01 | | | | 11*01 | | | | |
| | 1B1# | IGHV3- | IGHD6- | IGHJ4*02 | 9 | 17 | IGKV2- | IGKJ1*01 | 9 | 5 | 2 (2, 0, 0) |
| | | 21*01 | 19*01 | | | | 30*02 | | | | |
| | 2A1 | IGHV4- | IGHD7- | IGHJ4*02 | 10 | 14 | IGLV7- | IGLJ3*02 | 11 | 6 | 2 (0, 2, 0) |
| | | 31*03 | 27*01 | | | | 46*01 | | | | |
| | 1A10 | IGHV4- | IGHD3- | IGHJ4*02 | 15 | 20 | IGKV3- | IGKJ2*01 | 9 | 12 | 1 (0, 1, 0) |
| | | 34*01 | 10*01 | | | | 20*01 | | | | |
| | 1B8 | IGHV3- | IGHD2- | IGHJ4*02 | 10 | 19 | IGKV3- | IGKJ1*01 | 9 | 13 | 1 (0, 1, 0) |
| | | 7*01 | 2*01 | | | | 15*01 | | | | |
| | 1E9# | IGHV3- | IGHD6- | IGHJ4*02 | 18 | 18 | IGLV3- | IGLJ3*02 | 11 | 13 | 1 (0, 1, 0) |
| | | 11*06 | 13*01 | | | | 25*02 | | | | |
| | 1G12 | IGHV3- | IGHD6- | IGHJ4*02 | 18 | 15 | IGLV1- | IGLJ3*02 | 11 | 6 | 1 (0, 1, 0) |
| | | 11*06 | 19*01 | | | | 44*01 | | | | |
| KD11 (132) | 1E9 | IGHV1- | IGHD6- | IGHJ4*02 | 16 | 28 | IGKV2- | IGKJ5*01 | 9 | 6 | 2 (1, 1, 0) |
| | | 46*01 | 13*01 | | | | 30*02 | | | | |
| | 1C2 | IGHV1- | IGHD6- | IGHJ4*02 | 16 | 15 | IGKV2- | IGKJ5*01 | 9 | 5 | |
| | | 46*01 | 25*01 | | | | 30*01 | | | | |
| | 2A12 | IGHV3- | IGHD6- | IGHJ4*02 | 13 | 0 | IGKV3- | IGKJ2*01 | 10 | 0 | 2 (0, 2, 0) |
| | | 21*01 | 13*01 | | | | 20*01 | | | | |
| | 2H6 | IGHV3- | IGHD6- | IGHJ4*02 | 15 | 11 | IGLV9- | IGLJ3*02 | 13 | 4 | 2 (2, 0, 0) |
| | | 21/J4 | 6*01 | | | | 49*01 | | | | |
| | 1B7# | IGHV3- | IGHD2- | IGHJ6*02 | 20 | 1 | IGLV2- | IGLJ2*01 | 10 | 2 | 6 (0, 0, 6) |
| | | 23*01 | 2*01 | | | | 8*01 | | | | |
| | 1B12 | IGHV3- | IGHD3- | IGHJ6*02 | 25 | 10 | IGKV3- | IGKJ1*01 | 8 | 8 | 2 (2, 0, 0) |
| | | 49*03 | 3*01 | | | | 15*01 | | | | |
| | 2E7 | IGHV4- | IGHD3- | IGHJ5*02 | 18 | 15 | IGKV1- | IGKJ1*01 | 9 | 8 | 2 (2, 0, 0) |
| | | 4*02 | 10*01 | | | | 17*01 | | | | |
| | 1C1 | IGHV4- | IGHD3- | IGHJ6*02 | 14 | 1 | IGKV3- | IGKJ2*01 | 9 | 1 | 2 (0, 0, 2) |
| | | 34*01 | 10*01 | | | | 20*01 | | | | |
| | 1F6 | IGHV4- | IGHD1- | IGHJ5*02 | 18 | 16 | IGKV3- | IGKJ4*01 | 8 | 16 | 5 (5, 0, 0) |
| | | 39*07 | 7*01 | | | | 20*01 | | | | |
| | 2G1 | IGHV4- | IGHD1- | IGHJ5*02 | 18 | 17 | IGKV3- | IGKJ4*01 | 8 | 8 | |
| | | 39*07 | 7*01 | | | | 20*01 | | | | |
| | 2E10 | IGHV4- | IGHD3- | IGHJ3*02 | 16 | 16 | IGKV1- | IGKJ3*01 | 9 | 13 | 2 (2, 0, 0) |
| | | 59*01 | 3*01 | | | | 33*01 | | | | |
| | 2F12 | IGHV4- | IGHD3- | IGHJ3*01 | 16 | 20 | IGKV1- | IGKJ3*01 | 9 | 10 | |
| | | 59*01 | 3*02 | | | | 33*01 | | | | |
| | 1A12# | IGHV5- | IGHD1- | IGHJ6*02 | 20 | 7 | IGLV1- | IGLJ2*01 | 11 | 3 | 2 (0, 2, 0) |
| | | 51*01 | 7*01 | | | | 51*01 | | | | |
| | 1D6 | IGHV6- | IGHD5- | IGHJ4*02 | 13 | 0 | IGLV1- | IGLJ1*01 | 11 | 0 | 2 (0, 2, 0) |
| | | 1*01 | 18*01 | | | | 47*01 | | | | |
| | 2D5 | IGHV3- | IGHD3- | IGHJ4*02 | 22 | 11 | IGKV1- | IGKJ3*01 | 9 | 12 | 2 (2, 0, 0) |
| | | 23*01 | 10*01 | | | | 39*01 | | | | |

#Low yield of antibody prohibited further testing.

Expression of KD monoclonal antibodies—We preferentially made human light chain-rabbit heavy chain antibodies from the plasmablasts described in Table 2B, because they allowed for testing of the antibodies on human KD tissues using anti-rabbit secondary reagents. If sufficient quantities of antibody for testing were not produced using the rabbit heavy chain construct, fully human antibodies were made and biotinylated for immunohistochemistry assays. For four sets of clonally related sequences and one highly mutated IgA plasmablast, antibody production was not sufficient for further testing. For the other 38 clonally related sets and for the other antibodies made from mutated single IgA plasmablasts, approximately 300 ug to 1 mg of human/rabbit or human/human antibodies were produced in one 60 ml culture flask per assay. We tested 60 monoclonal antibodies from 11 patients. Of the 60 antibodies, 52 had entirely different VH/VL sequences and eight were members of clonally related plasmablast sets in which the related antibodies had 1-4 amino acid mutations in the CDR3 sequence within the set.

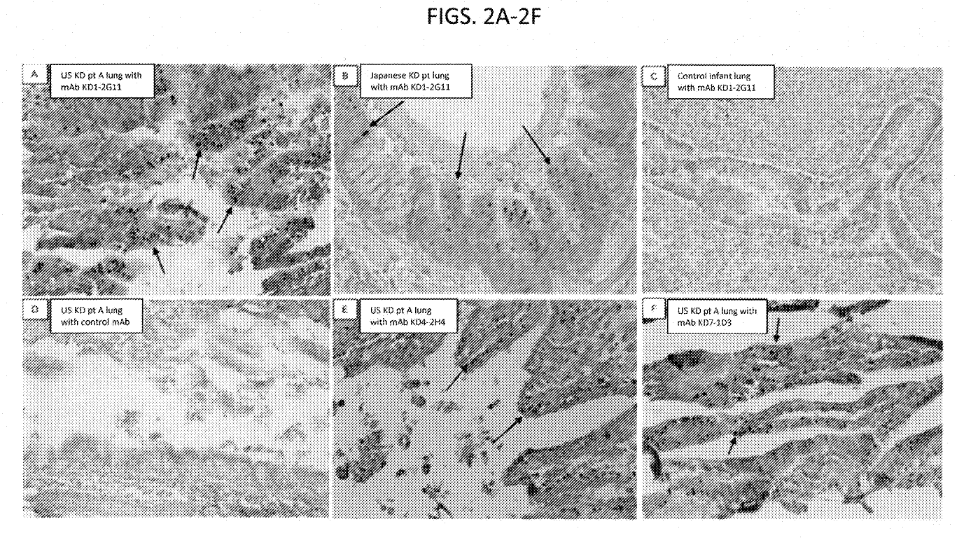

Immunohistochemistry shows binding of KD monoclonal antibodies to cytoplasmic inclusion bodies in KD ciliated bronchial epithelium—Our prior studies demonstrated binding of synthetic antibodies with non-cognate VDJ and VJ pairs to intracytoplasmic inclusion bodies in ciliated bronchial epithelial cells of children who died from acute KD but not of infant controls who died of non-KD illnesses (29-31, 35). We therefore initially performed immunohistochemistry assays to determine if the newly synthesized KD monoclonal antibodies with in vivo cognate VDJ and VJ partners identified the inclusion bodies in acute KD formalin-fixed, paraffin-embedded lung tissue. Failure to bind in this assay does not preclude synthetic antibodies from being KD antigen-specific, because antigens can be disrupted by formalin fixation and paraffin embedding. However, strong positive results from this assay were useful to prioritize antibodies for additional assays that could specifically identify target KD antigen(s). Initial studies with antibodies KD1-2G11 and KD4-2H4 revealed strong binding of the antibody to KD lung tissues from the United States (n=3) and Japan (n=2) (FIG. 2A, B) and not to infant control lung tissue (n=3) (FIG. 2C), similar to our prior studies (29-31, 35, 36). Because tissue samples from fatal KD cases are very scarce and the available samples virtually always consist of small amounts of tissue in formalin-fixed, paraffin-embedded archived autopsy blocks, it was not possible to test each antibody on multiple KD patient tissues. Instead, tissues from a KD child found in our prior studies to have many inclusion bodies in ciliated epithelium of medium-sized bronchi and from whom we had multiple lung tissue blocks were used to test all 60 monoclonal antibodies for inclusion body binding. For each antibody tested, we recorded a strong positive result when inclusion bodies were seen with the low power 4× or 10× objectives, a weak positive result when inclusion bodies were only observed with the 20× or 40× objectives, and a negative result when inclusion bodies could not be observed using the 40× objective (Table 1C and 1D). Strong positive staining of intracytoplasmic inclusion bodies in KD ciliated bronchial epithelium similar to that demonstrated in our prior studies (29-31, 35, 36) was observed using 10 monoclonal antibodies: KD1-2G11, KD4-2H4, KD6-1A10, KD6-2B2, KD7-1D3, KD7-2C1, KD9-1E10, KD11-1C2, KD11-1E9, and KD11-2E10 (Table 1C and 1D, FIGS. 2A,B,E,F). Most of these strongly binding antibodies were from clonally related sets of plasmablasts, but KD7-1D3 (FIG. 2F), KD7-2C1, and KD9-1E10 were from single IgA plasmablasts with heavy chain sequences that had 13-22 amino acid mutations compared to germline. With the exception of KD11-1E9 and KD11-1C2, which are clonally related antibodies from the same patient, all of the strongly binding antibodies were genetically different (Table 2C). Overall, 32/60 (53%) KD monoclonal antibodies detected intracytoplasmic inclusion bodies in KD ciliated bronchial epithelium. Of antibodies made from the clonally related sets of plasmablasts, 26/46 (57%) yielded a positive result, while 6/14 (43%) antibodies made from selected single IgA plasmablasts showing 13-22 amino acid mutations from germline yielded a positive immunohistochemistry result. Inclusion bodies were not demonstrated in KD bronchial epithelium with adult control monoclonal antibodies (FIG. 2D). Overall, an antibody that recognizes KD inclusion bodies was made from 9/11 (82%) KD patients sampled. The two patients from whom we did not clone such an antibody were sampled at the latest time points after onset in the cohort, on days 20 and 24 after fever onset, at which time the antigen-specific plasmablast response may have abated (32). A common antibody produced among the nine patients was not observed. Instead, the antibodies that identified KD inclusion bodies were genetically different between the patients (Table 2C).

TABLE 2C
Genetic characteristics of Kawasaki disease (KD) monoclonal antibodies
(Mab) that bind strongly to KD intracytoplasmic inclusion bodies.

| | | | #mutations | | | |
|---|---|---|---|---|---|---|
| | | CDR3 | from | | | |
| | | length | germline | | KD | Original |
| Mab | Heavy chain | (amino acids) | (amino acids) | Light chain | patient | isotype |
| 1-2G11 | IGHV3-15/JH4 | 15 | 10 | IGKV1-30/JK3 | 1 | IgA |
| 4-2H4 | IGHV3-74/JH2 | 14 | 4 | IGLV2-14/JL3 | 4 | IgG |
| 6-1A10 | IGHV3-33/JH4 | 20 | 11 | IGLV1-44/JL3 | 6 | IgA |
| 6-2B2 | IGHV3-33/JH3 | 12 | 15 | IGKV1-5/JK1 | 6 | IgA |
| 7-1D3 | IGHV3-23/JH4 | 11 | 15 | IGKV1-5/JK1 | 7 | IgA |
| 7-2C1 | IGHV3-23/JH4 | 14 | 13 | IGLV5-45/JL3 | 7 | IgA |
| 9-1E10 | IGHV1-46/JH4 | 12 | 22 | IGLV1-44/JL1 | 9 | IgA |
| 11-1E9* | IGHV1-46/JH4 | 16 | 28 | IGKV2-30/JK5 | 11 | IgG |
| 11-1C2* | IGHV1-46/JH4 | 16 | 15 | IGKV2-30/JK5 | 11 | IgA |
| 11-2E10 | IGHV4-59/JH3 | 16 | 16 | IGKV1-33/JK3 | 11 | IgG |

*Clonally related plasmablasts from KD patient 11

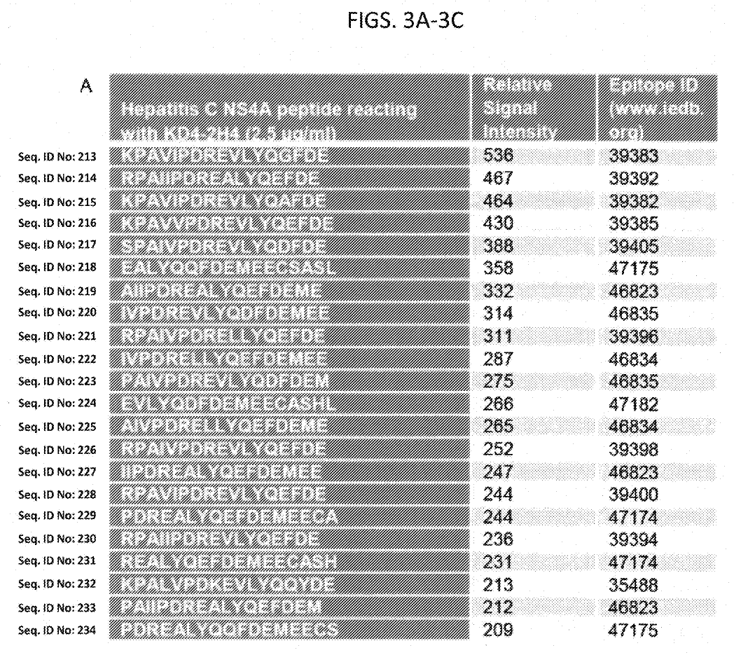

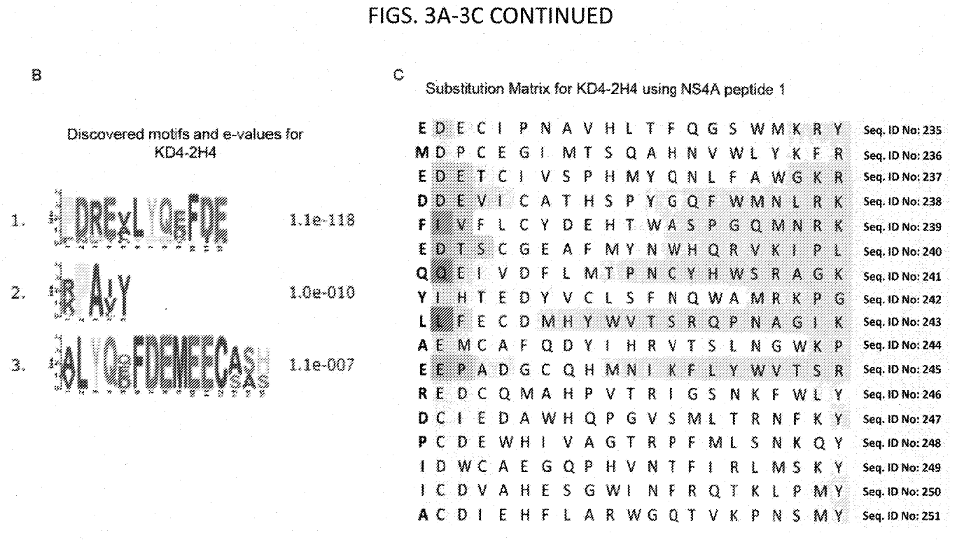

Screening an animal virus peptide array reveals KD4-2H4 recognizes hepacivirus peptides—To determine whether KD monoclonal antibodies bind to an epitope that is shared with a known animal virus, we created a custom array (PEPperPRINT, Table S4) of peptides reported to be B cell epitopes of animal viruses and included in the Immune Epitome Database and Analysis Resource (available on the World Wide Web at iedb.org). Monoclonal antibody KD4-2H4 showed binding to multiple similar peptides from a short region of the C-terminal end of the NS4A protein of hepacivirus C (FIG. 3A). MEME bioinformatic analysis was used to identify a shared motif among the top 27 peptide hits with spot intensities of more than 200 fluorescence units. This yielded three discovered motifs, with the highest significant at 1.1e-118 (FIG. 3B). Motifs 1 and 3 are partially overlapping and the second motif is just upstream of motif 1 in hepacivirus C NS4A. To be certain that patient KD4 was not co-incidentally infected with hepatitis C virus, we performed a fourth generation enzyme linked immunosorbent assay (ELISA) on KD4 patient serum for antibody to hepatitis C (Abnova KA2534). Hepatitis C antibody was not detected in this child's serum but was detected in positive control serum. A positive result in this ELISA requires the presence of antibodies to hepatitis C NS3, NS5, and core proteins, and the negative result obtained indicates that these antibodies were not present in this child's sera. These results suggest but do not prove that the KD antigen is a virus that shares some homology with an epitope present in the NS4A protein of hepacivirus C, but may otherwise not be closely related to this virus

Substitution matric analysis demonstrates amino acids required for antibody KD4-2H4 binding to a hepacivirus peptide—To determine critical amino acids for KD4-2H4 binding to hepacivirus NS4A peptide 1, we tested a peptide substitution array, in which each position of the reactive peptide AIIPDREALYQEFDEME (SEQ ID NO:219) was sequentially replaced by each of 20 amino acids (PEPperPRINT). This peptide was chosen from the reactive peptides on the animal virus peptide array to place the highly significant motif LYQxFDE in the mid-region of the peptide. The substitution array showed that amino acids 9L and 11Q of this peptide were essential for antibody binding (FIG. 3C). Replacement of 12E by D, T, or S and of 13F by I and V resulted in a marked increase in antibody binding on the array. These results indicate that antibody KD4-2H4 shows optimal binding to a peptide sequence similar to, but distinct from, the hepacivirus C peptides on the animal virus peptide array. It also demonstrates that the hepacivirus C homology corresponds to an ˜8 amino acid segment of the NS4A region, similar to the length of other linear epitopes bound by antibodies (37). We have not detected an RNA genome with genomic similarity to hepacivirus C in a high-throughput RNA sequencing dataset we are investigating from patient sera (n=11), pooled throat swabs (n=4), liver (n=1), coronary artery (n=8) (38), heart (n=4), lung (n=10), and spleen (n=1) tissues [data not shown].

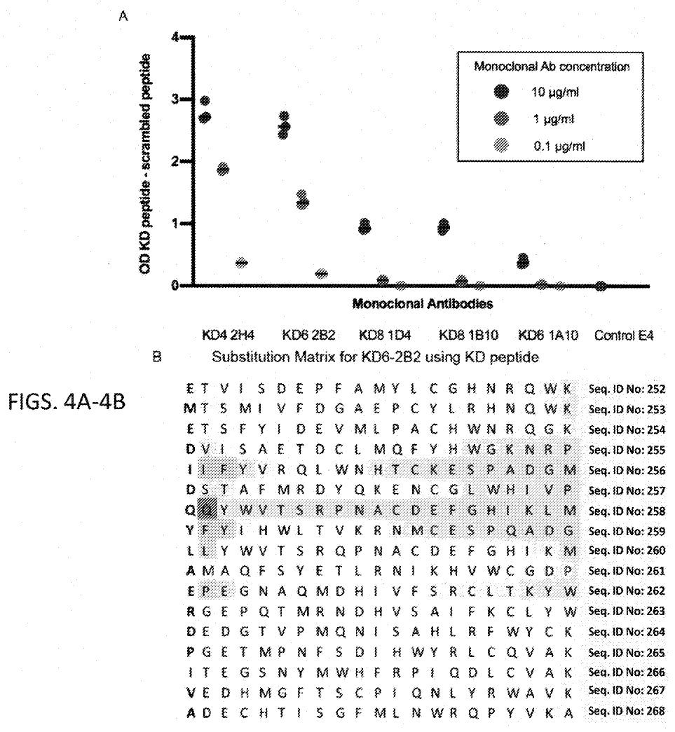

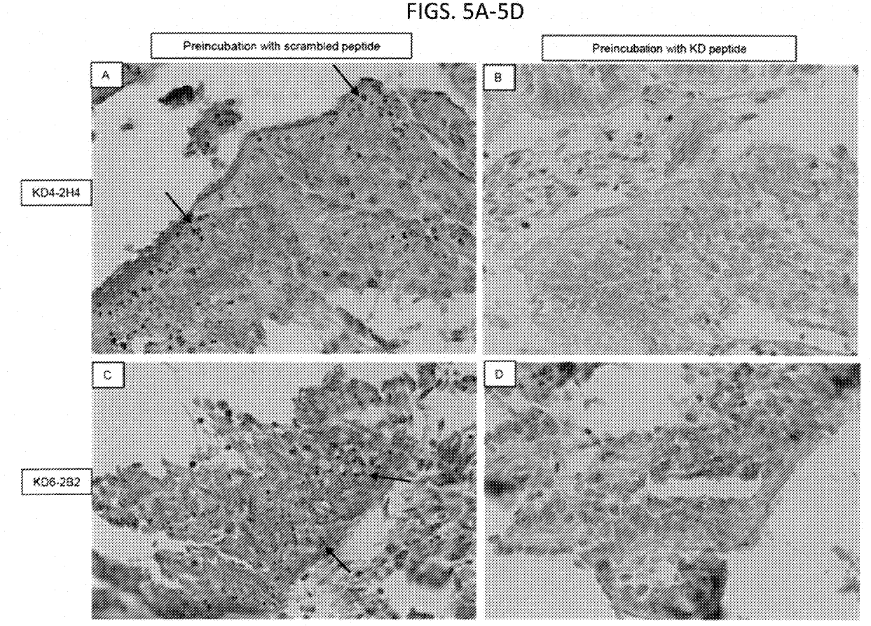

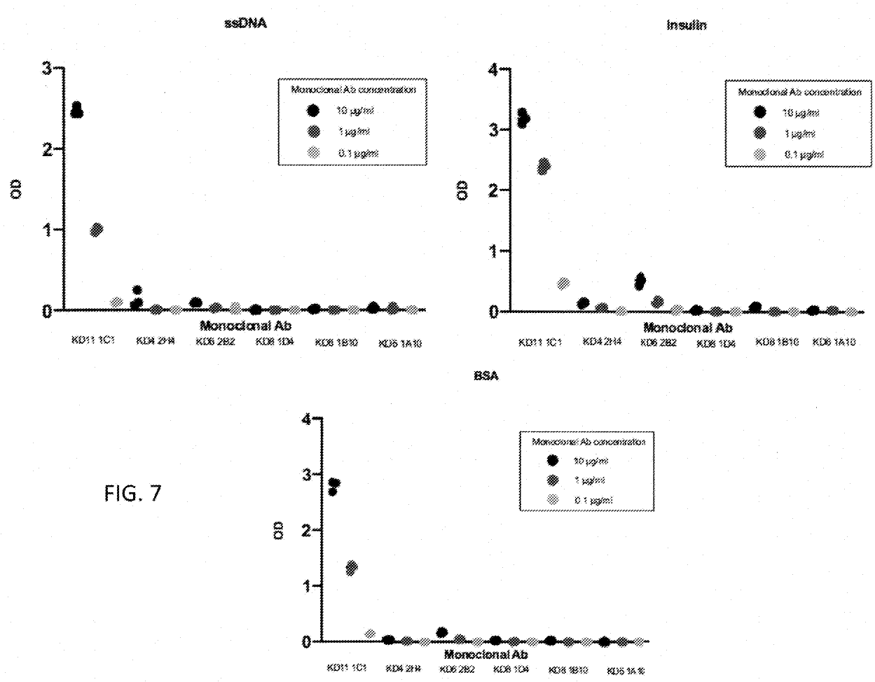

Multiple KD monoclonal antibodies recognize an optimized KD peptide by ELISA—Initial ELISA experiments showed that monoclonal antibody KD4-2H4 reacted with hepacivirus C NS4Apeptide 1. This peptide was derived from the strongest binding peptide on the animal virus peptide array (FIG. 3A,B). Following from the substitution matrix analysis, we prepared KD peptide (AVIPDREALYQDIDEME, SEQ ID NO:212), which includes three amino acid substitutions compared to the original reactive peptide, and conjugated this new peptide to bovine serum albumin to improve microtiter well coating (Table 3). We also prepared a similarly conjugated scrambled peptide as a control, and tested all 60 monoclonal antibodies for binding to KD peptide and scrambled peptide. Monoclonal antibodies KD4-2H4, KD6-2B2, KD6-1A10, KD8-1B10, and KD8-1D4 reacted with KD peptide and not with the scrambled peptide by ELISA (FIG. 4A). Monoclonal antibodies KD8-1B10 and KD8-1D4 are members of a set of clonally related antibodies with one amino acid difference in CDR3. Other than these two clonally related antibodies derived from the same patient, the antibodies showing KD peptide binding were genetically different (Table 4). The remainder of the KD monoclonal antibodies did not show specific binding to KD peptide by ELISA. All of the antibodies reactive with the peptide by ELISA identify inclusion bodies in KD ciliated bronchial epithelium by immunohistochemistry. Therefore, of the nine KD patients in this study whose antibodies identified KD inclusion bodies, 3 patients (33%) had plasmablasts with differing VDJ and VJ sequences (Table 4, Tables 1C and 1D) that recognize KD peptide. These antibodies were not polyreactive against DNA, insulin, or bovine serum albumin by ELISA (FIG. 7).

TABLE 3
C-terminal region amino acid sequences of some hepaciviruses and
comparison with the NS4A peptides recognized by KD monoclonal antibodies.

| Hepacivirus | NS4A sequence | SEQ | Accession |
|---|---|---|---|
| Hepacivirus C (human) | KPAVIPDREVLYQAFDEMEEC | 193 | AFX78101 |
| Hepacivirus A (non-primate, | KVVVVPHKGVLYADFDEMEEC | 194 | KP325401 |
| canine, equine) | | | |
| Hepacivirus K (bat) | KIFISQDRDNLYTILDEMEEC | 195 | KC796074 |
| Hepacivirus M (bat) | KIFLSPDKDHLYGWFEEMEEC | 196 | KC796078 |
| Hepacivirus L (bat) | TTLEDADMPAYLTDMGELEEC | 197 | KC796077 |
| Hepacivirus E (rodent) | TTAARVVQPPEDDITENLEEC | 198 | KC815310 |
| Hepacivirus F (rodent) | STAARVVEFEPLDTEEVLEEC | 199 | KC411784 |
| Hepacivirus J (rodent) | FVNTLPPEVLFELEPTEAEEC | 200 | KC411777 |
| Hepacivirus I (rodent) | CRPELRLNETIDEPAIELEEC | 201 | KC411806 |
| Hepacivirus G (Norway rat 1) | DSSAALNPAPEMSILEEVEEC | 202 | MF113386 |
| Hepacivirus H (Norway rat 2) | AKGAEPXPLEQEMDTAGMEEC | 203 | KJ950939 |
| Hepacivirus N (bovine) | DSGEPPNAPEPADDDDGFEEC | 204 | MH027953 |
| Hepacivirus D (Guereza, Old | QAERAQVMEDFIGDLIETEEC | 205 | KC551800 |
| World monkey) | | | |
| Sifaka hepacivirus (lemur) | TTSALHAPPPPISLEGALEEC | 206 | MH824539 |
| Hepacivirus B (GB virus-B; | SVPTGATVAPVVDEEEIVEEC | 207 | U22304 |
| human) | | | |
| Hepacivirus P (ground squirrel) | STASLASQPIDDPLTSGLEEC | 208 | MG211815 |

Peptides used in this study

| | | |
|---|---|---|
| NS4A peptide 1 | KPAVIPDREVLYQGFDEMEEC | 209 |
| NS4A peptide 2 | KPAVIPDREALYQDIDEMEEC-BSA conjugated | 210 |
| Scrambled peptide | IYPLEDMAEPKVERIDAQEDC-BSA conjugated | 211 |

TABLE 4
Genetic characteristics of Kawasaki disease (KD) monoclonal
antibodies (Mab) that recognize KD peptide

| | | | #mutations | | | | Binding |
|---|---|---|---|---|---|---|---|
| | | CDR3 | from | | | | to KD |
| | Heavy | length | germline | Light | KD | Original | inclusion |
| Mab | chain | (aacids) | (aacids) | chain | patient | isotype | bodies |
| 4-2H4 | IGHV3- | 14 | 4 | IGLV2- | 4 | IgG | Strong |
| | 74/JH2 | | | 14/JL3 | | | |
| 6-1A10 | IGHV3- | 20 | 11 | IGLV1- | 6 | IgA | Strong |
| | 33/JH4 | | | 44/JL3 | | | |
| 6-2B2 | IGHV3- | 12 | 15 | IGKV1- | 6 | IgA | Strong |
| | 33/JH3 | | | 5/JK1 | | | |
| 8-1B10* | IGHV3- | 12 | 3 | IGKV1- | 8 | IgA | Weak |
| | 72/JH3 | | | 6/JK4 | | | |
| 8-1D4* | IGHV3- | 12 | 1 | IGKV1- | 8 | IgA | Weak |
| | 72/JH3 | | | 6/JK4 | | | |

*Clonally related plasmablasts from KD patient 8

Monoclonal antibodies KD4-2H4 and KD6-2B2 share a common epitope—KD36-2B32 showed strong reactivity with KID peptide by ELISA (FIG. 4A), indicating that it likely binds to a similar epitope as KD4-2H4. To determine if these antibodies recognize the same epitope, we performed a substitution matrix (PEPperPRINT), which revealed that KD6-2B2 binds to an epitope highly similar to that of KD4-2H4 (FIG. 4B).

Blocking experiments show that the KD peptide epitope is present in KD inclusion bodies—Pre-incubation of monoclonal antibodies KD4-2H4 and KD6-2B2 with KD peptide blocked binding of the antibodies to intracytoplasmic inclusion bodies in KD ciliated bronchial epithelium (FIG. 5B,D), demonstrating that an epitope of this peptide sequence is a specific target of the antibodies. Incubation with the scrambled peptide did not block the binding (FIG. 5A,C). These results demonstrate that a protein with an epitope highly similar to KD peptide is present in the inclusion bodies.

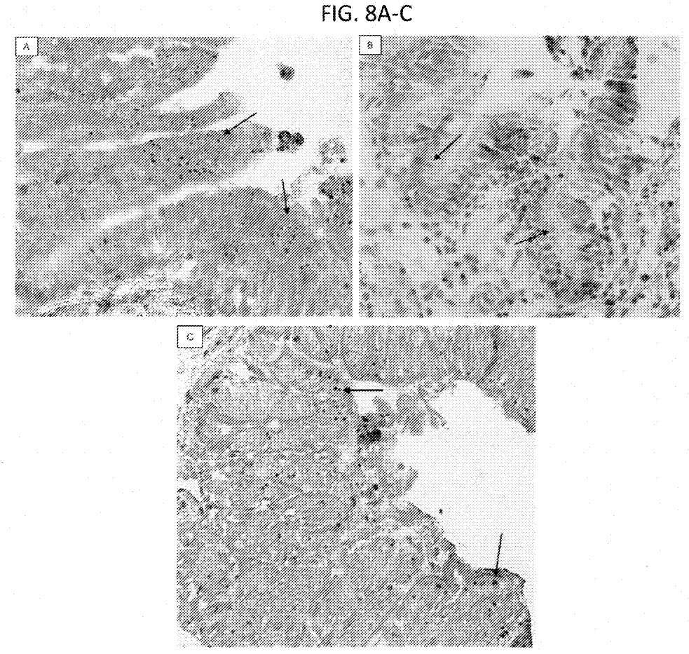

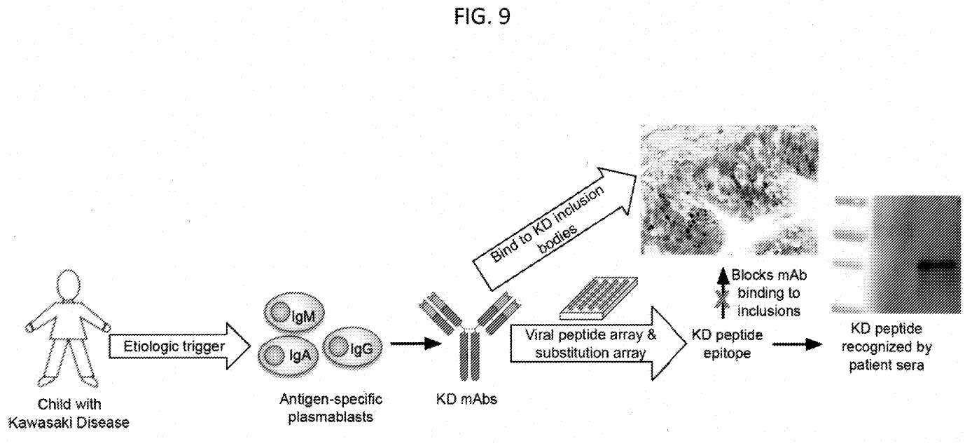

Human protein array analyses and immunohistochemistry studies of monoclonal antibodies KD4-2H4 and KD6-2B2 do not yield a human protein as the target of the antibodies m—We performed human protein array analysis using both KD4-2H4 and KD6-2B2. The array covers ˜80% of the canonical proteome. The only human protein that showed reactivity with both antibodies on this array was integral membrane protein 2B (ITM2B), a transmembrane protein whose C-terminal end is processed by proteases to release a peptide that inhibits the deposition of beta-amyloid; mutations in this protein are associated with neurodegenerative diseases (39). This reactivity could be explained by a partially shared epitope between KD peptide and the ITM2B protein (-ALYQ-I-, (SEQ ID NO: 192)). Immunohistochemistry of KD lung with polyclonal anti-ITM2B revealed a different pattern of staining of bronchial epithelium compared with KD4-2H4 and KD6-2B2, and anti-ITM2B did not block the binding of the antibodies to inclusion bodies in KD ciliated bronchial epithelium (FIG. 8). These results strongly suggest that ITM2B is not the antigen in inclusion bodies in KD ciliated bronchial epithelium specifically targeted by these KD monoclonal antibodies. These results do not exclude a potential human antigen as the target of KD; however, the clinical and epidemiologic features of the illness do not support this explanation of disease pathogenesis (40).

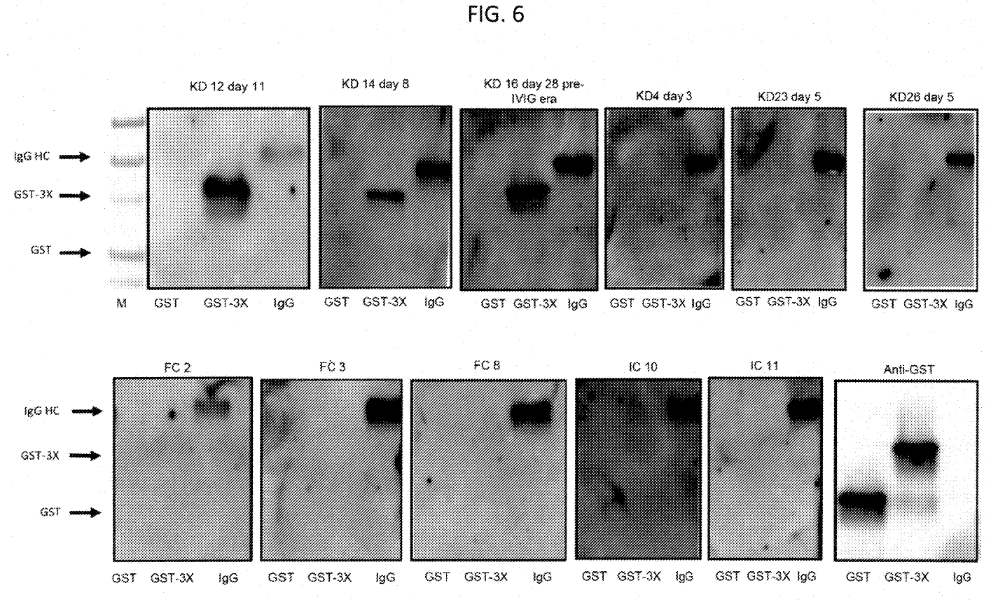

Sera from additional KD patients recognize KD peptide—To determine if sera from KD patients recognize KD peptide, we performed Western blot analyses for IgG antibody using glutathione sulfur transferase (GST)-KD peptide multimer fusion protein and GST as the antigens. We screened KD and control patient sera at a dilution of 1:5000 in phosphate buffered saline, which reduced background from non-specific binding. A minority of sera (both KD and control) exhibited non-specific binding (reactivity with GST alone) and were excluded from the study. Treatment of KD children with intravenous gammaglobulin (IVIG) at the time of diagnosis could potentially complicate analysis for IgG seroconversion to the identified epitope. Therefore, we tested sera from KD patients who presented on days 8-14 after fever, prior to receiving IVIG therapy (Table 5), when IgG antibody to the agent might have developed. We also tested serum taken on day 28 after onset from a KD patient who presented prior to the time that IVIG was standard treatment for KD. These results showed that sera from 5/8 KD children who had not received IVIG therapy had IgG antibody to the KD peptide epitope during or after the second week of illness (Table 5, FIG. 6). Patient KD12, whose serum gave a particularly strong signal at 1:5000 on day 11 after fever onset and before IVIG therapy, was found to be seropositive in serial dilutions as high as 1:50,000. Because clinical and epidemiologic features of KD strongly suggest a ubiquitous infectious agent, the most appropriate control group for serologic studies is infants at 5-9 months of age without KD, an age when passive maternal antibody would have abated and a low likelihood of prior infection from a ubiquitous agent would be expected. These results showed that sera from 0/17 infant controls had IgG antibody to the KD peptide epitope (Table 6, FIG. 6) compared with 5/8 KD sera at ≥day 8 of illness (p<0.01). We hypothesized that IVIG would contain antibody to the epitope, since adult donors whose blood is used to prepare this product would likely already have experienced infection with a ubiquitous agent, and the IVIG product and lot that we tested gave indeterminate results, because it appeared to react to GST alone as well as to the GST-KD peptide fusion protein. We also tested sera from two additional groups of children. Sera from 1/9 febrile control children ages 7 months to 10 years had IgG antibody to the epitope identified; the positive result was in a 5 yr old patient, who may have had sufficient IgG antibody for detection at a 1:5000 dilution from relatively recent asymptomatic infection with the putative agent. We also tested sera from KD patients who presented on day 4-5 of fever, at which time we hypothesized that an IgG response to the triggering antigen might not yet have developed, and IgG antibody was detected in 1/11 KD children in this group. Sera from patient KD30 was positive for IgG antibody at day 4 after fever onset. The parents of this child reported that the child was ill with neck stiffness, abdominal pain, photophobia, and arthralgias 10 days before admission, although they had only recognized fever for the preceding 4 days. Upon admission, the child was recognized to have all clinical features of KD. This raises the possibility that the duration of KD was longer than one week at admission, although early development of an IgG response is also possible. Pre-treatment sera at or before day 5 of illness, when IgG antibody might not yet have developed in response to the KD agent, was available from only one of the 11 patients whose plasmablasts were studied. Sera from day 3 of illness on KD patient 4, whose day 8 of illness IgG plasmablast-derived antibody KD4-2H4 recognized the protein epitope, was negative by Western blot assay, suggesting development of antibody to this epitope between days 3 and 8 of illness (Table 5). There are many possible reasons for the lack of detection of IgG antibody in 3/8 patients on day 8-9 after illness onset. IgG antibody may not be detectable by this time in some patients or might be more easily detected at a lower serum dilution. KD could have been misdiagnosed in the patient who did not develop coronary aneurysms, as the development of these aneurysms is presently the only way to confirm the diagnosis. Other possible explanations for negative results in this assay are that KD could result from other etiologic triggers or result from multiple serotypes of the etiologic agent that do not cross-react and therefore are not detected in this assay. When considered together, these serologic assay results support the likelihood that a ubiquitous infectious agent containing the identified epitope is etiologically related to KD.

TABLE 5
Kawasaki Disease (KD) patient sera (before IVIG treatment) tested
by Western blot assay for IgG antibody to KD peptide multimer.

| | | | | Day of | Coronary | Western | |
|---|---|---|---|---|---|---|---|
| Sample | Age | | Gender | Illness | Abnormalities | result | Year |
| KD12 | 10 | yr | M | 11 | Yes | Positive | 1994 |
| KD13 | 4 | yr | F | 14 | Yes | Positive | 2003 |
| KD14 | 6 | yr | F | 8 | Yes | Positive | 2003 |
| KD15 | 3 | yr | M | 9 | No | Positive | 2002 |
| KD16 | 6 | mo | M | 28 | Yes | Positive | 1984 |
| KD17 | 3 | yr | M | 8 | No | Negative | 2002 |
| KD18 | 15 | yr | M | 9 | Yes | Negative | 1991 |
| KD19 | 3 | yr | M | 8 | Yes | Negative | 2011 |
| KD4 | 3 | mo | M | 3 | Yes | Negative | 2017 |
| KD20 | 7 | mo | F | 5 | Yes | Negative | 2013 |
| KD21 | 7 | mo | F | 5 | Yes | Negative | 2005 |
| KD22 | 3 | yr | F | 5 | Yes | Negative | 2002 |
| KD23 | 2 | yr | F | 5 | No | Negative | 2008 |
| KD24 | 16 | mo | M | 5 | No | Negative | 2009 |
| KD25 | 20 | mo | M | 5 | No | Negative | 2017 |
| KD26 | 17 | mo | M | 5 | No | Negative | 2017 |
| KD27 | 4 | mo | F | 4 | No | Negative | 1991 |
| KD28 | 3 | yr | M | 4 | No | Negative | 1991 |
| KD29 | 2 | yr | M | 4 | No | Negative | 1991 |
| KD30 | 7 | yr | F | 4 | No | Positive | 1990 |

TABLE 6
Control sera tested by Western blot assay
for IgG antibody to KD peptide multimer.

| | | | | | Western |
|---|---|---|---|---|---|
| Sample | Age | | Gender | Diagnosis | result |
| Febrile control 1 | 9 | yr | F | Staphylococcus | Negative |
| | | | | aureus septic | |
| | | | | arthritis | |
| Febrile control 2 | 7 | mo | M | Haemophilus | Negative |
| | | | | influenzae type a | |
| | | | | meningitis | |
| Febrile control 3 | 11 | mo | M | Staphylococcus | Negative |
| | | | | aureus septic | |
| | | | | arthritis | |
| Febrile control 4 | 21 | mo | M | Group A | Negative |
| | | | | streptococcal | |
| | | | | septic arthritis | |
| Febrile control 5 | 14 | mo | F | E. coli | Negative |
| | | | | pyelonephritis | |
| Febrile control 6 | 10 | yr | F | Staphylococcus | Negative |
| | | | | aureus | |
| | | | | osteomyelitis | |
| Febrile control 7 | 4 | yr | F | Viral-induced | Negative |
| | | | | leukopenia and | |
| | | | | thrombocytopenia | |
| Febrile control 8 | 4 | yr | F | Pneumonia with | Negative |
| | | | | effusion | |
| Febrile control 9 | 5 | yr | M | Pneumococcal | Positive |
| | | | | pneumonia | |
| Infant | 5-9 | mo | Unknown | Unknown | N = 17 |
| controls 10-26 | | | | | Negative |
| (deidentified) | | | | | |

### Discussion

Here, we identified a peptide that is recognized by antibodies that develop during acute KD. Our data demonstrate that monoclonal antibodies derived from clonally expanded plasmablasts in the peripheral blood of children with KD identify intracytoplasmic inclusion bodies in the bronchial epithelium of fatal KD cases. A subset of these antibodies bind to a specific protein epitope that is similar to one found in the NS4A protein of hepacivirus C. Furthermore, the inclusion bodies contain a protein with this epitope. We also demonstrate that IgG antibody to this epitope is much more prevalent during or after the second week of illness than in the first week of KD, and we did not detect antibody in 17 infants 5-9 months of age. Our report of an epitope targeted by the immune response to KD demonstrates that preparing antibodies from plasmablast responses to acute inflammatory diseases of unknown etiology can be useful in identifying the inciting antigens.

Whether the protein epitope identified in this study derives from a previously unidentified hepacivirus or related virus remains to be determined. Although some propose an autoimmune etiology of KD, the marked severity of the disease in very young infants (41) and the peak incidence at 10 months of age (42) are not supportive of this hypothesis, because autoimmune diseases rarely arise in the first year of life. Moreover, the short 1-3 week duration of the clinical illness even in untreated patients (3) and the rarity of disease recurrence (43) argue against the autoimmune theory. In contrast, many lines of evidence support a ubiquitous viral agent as the cause of KD in genetically susceptible children. These include the young age group affected (42), well-documented epidemics of illness (2, 44-46), the self-limited nature of the clinical illness (3), the lack of clinical response to antibiotic therapy (3, 43), the high prevalence of the condition in Japan, where 1 in 65 children develop KD by the age of 5 (42) (a prevalence rate similar to that of many ubiquitous viral infections), the upregulation of interferon response genes in KD lung and coronary arteries (35, 38), the identification of virus-like particles in close proximity to KD inclusion bodies in ciliated bronchial epithelium (35), and the prominent IgA immune response suggesting a mucosal portal of entry of the putative ubiquitous causative agent (28, 30, 47, 48).

Hepaciviruses are enveloped, spherical RNA viruses that are ˜50 nm in diameter and can result in persistent infection. Since 2010, when hepatitis C virus was the sole confirmed member of the Hepacivirus genus, there has been a steady increase in the number of new hepaciviruses identified in various animal species (49). The identified epitope recognized by the KD monoclonal antibodies reported here is most similar to that of hepacivirus C (˜90% identity), non-primate hepacivirus (˜60% identity), and hepacivirus M and K (bat, ˜50% identity) and less homologous to that of rodent, bovine, Old World monkey, and lemur hepaciviruses (Table 3). It differs substantially from the NS4A sequence of hepacivirus B (GB virus-B) and pegiviruses.

Although hepaciviruses are primarily hepatotropic, RNA of non-primate hepacivirus has been identified in the cytoplasm of ciliated bronchial epithelial cells of infected dogs and horses by in situ hybridization (50, 51). Our prior studies indicated that KD intracytoplasmic inclusion bodies contain RNA and can persist in at least a subset of KD patients (36). Moreover, using transmission electron microscopy, we found that inclusion bodies in KD ciliated bronchial epithelial cells were located in the endoplasmic reticulum/Golgi region, with spherical, 50 nm virus-like particles in close proximity (35), consistent with but not confirmatory of infection with a virus in this family or one with a viral ancestry shared with the hepaciviruses. However, the epitope identified is short, and could derive from a non-hepacivirus source.

We are presently using genomic and proteomic approaches to determine the gene sequence from which the immunogenic epitope identified in this study arises. We hypothesize that the source is an RNA virus whose genome is present in very low quantity in KD clinical samples, such as blood at the time of clinical presentation, and in the target tissues of fatal cases by the time of death occurring weeks to years after illness onset. We are working to identify the targets of KD monoclonal antibodies that bind ICI but do not bind to KD peptide, because these antibodies may bind other epitopes of the putative viral agent. We are also developing more sensitive assays for multiple antibody isotypes that could facilitate KD diagnosis and could be evaluated in worldwide multicenter studies.

This study is limited by its investigation of the plasmablast response to KD in a single US center over a 16-month period from 2017-18. However, KD monoclonal antibodies prepared in this study react with tissue samples from KD children from other geographic areas of the US and Japan who died during different decades, and sera from KD children in Chicago from the 1980s, 1990s and 2000s react with the identified protein epitope, suggesting that the results may be applicable to additional KD patients in other locations and over other time periods.

Further multicenter collaborative studies are needed to investigate the relevance of these findings to KD patients around the world. Despite these limitations, we believe our results may provide the most promising direction for etiologic studies of KD and the development of serologic tests that has emerged in the more than 50 years since Dr. Kawasaki described the clinical features of the disease (52). Identification of the etiology of KD is a pediatric research priority that will enable diagnostic test development, improved therapy, and ultimately prevention of this serious, increasingly recognized worldwide childhood illness.

### Methods

Patients and specimens. Peripheral blood (3 ml) was obtained from 11 KD patients on day 8-24 after fever onset (FIG. 1A, Table 2A) from April 2017 through July 2018. Seven patients had coronary artery aneurysms and four did not. Four patients with incomplete KD as defined by American Heart Association criteria (3) were included; two had coronary artery aneurysms (KD 4 and 8, Table 2A), one with congenital heart disease initially was thought to have coronary dilation by echocardiography but computed tomographic angiography revealed normal coronary arteries (KD3), and one did not develop coronary abnormalities (KD5). All KD patients were treated with intravenous gammaglobulin and aspirin therapy, and all but one (KD2, diagnosed and treated initially at another institution) were deemed high-risk because of age, presence of coronary artery aneurysms at diagnosis, and/or a laboratory profile of severe inflammation, and were given primary adjunctive therapy with prednisone. (3, 53) Tissue samples from KD patients were obtained over the last several decades from the US and Japan and were de-identified. Blood was also obtained from one healthy adult volunteer as a source of control antibodies.

Flow cytometry. Peripheral blood mononuclear cells obtained by Ficoll gradient centrifugation were stained with antihuman CD3-FITC (Fisher), CD19-Pacific Blue, CD38-AlexaFluor647, and CD27-PE (Biolegend). CD3-CD19+CD38++CD27++ cells were gated (FIG. 1A) and single cells sorted into individual wells of 96-well PCR plates on a BD FACSAria SORP system. Sera available from additional KD children were used for serologic assays (Table 5), as were sera from febrile control children and infant controls (Table 6). The infant sera were de-identified samples stored from patients 5-9 months of age who were tested and found to be negative for human immunodeficiency virus infection. The infants were cared for in the inpatient wards and outpatient clinics at Lurie Children's and likely included children who were well and those who were ill with various medical problems, but specific diagnoses were not available.

Amplification, sequencing and cloning of immunoglobulin variable regions. Reverse transcription and PCR of heavy and light chain variable genes were performed according to a published protocol (54, 55), and PCR products were directly sequenced. Heavy chain sequences were analyzed for VH family and CDR3 sequence and clonally related sequences identified and prioritized for antibody synthesis. Plasmablasts were determined to be clonally related when their sequences encoded identical functional heavy and light chain variable families and had a CDR3 sequence of identical length with the same amino acid sequence or 1-4 amino acid differences. Sequences that were out-of-frame or had stop codons were not further investigated. In some cases, IgA plasmablasts showing substantial somatic mutation of their immunoglobulin variable region sequences were prioritized for production, even if other clonally related plasmablasts were not identified in the dataset from that patient. Light chains were cloned into human immunoglobulin kappa or lambda light chain expression vectors (55) and heavy chains were cloned into human gamma1 and rabbit gamma (pFUSEss vectors, InvivoGen, San Diego, Calif.) heavy chain expression vectors, to enable production of human and rabbit versions of the antibodies.

Antibody production. Antibodies were produced by transfection of 293F suspension cells using a 1.5:1 ratio of light chain:heavy chain DNAs and Freestyle MAX reagent, and were purified using protein A agarose beads (ThermoFisher Scientific).

Immunohistochemistry using KD monoclonal antibodies. Immunohistochemistry was performed on KD and control infant tissues as previously reported (29, 47) using a screening dilution of 1:100 of each synthetic antibody and either biotinylated human antibody or rabbit antibody followed by biotinylated goat anti-rabbit secondary antibody using the Vectastain ABC Elite kit (Vector Laboratories). If background staining was high at 1:100, the antibodies were further diluted.

Monoclonal antibody reactivity with animal virus peptides. A custom Animal Virus Discovery peptide array was designed (PEPperPRINT, available on the World Wide web at pepperprint.com) made up of 29,939 peptides derived from 13,123 B cell animal virus epitopes reported in the Immune Epitope Database and Analysis Resource (available on the World Wide Web at iedb.org), control hemagglutinin peptides (769 spots), and 1,261 random peptides to fill the microarray. Peptides >4 amino acids were printed either in full length or translated into overlapping 17 amino acid peptides and were printed in duplicate. The array was blocked with blocking buffer MB-070 (Rockland) for 30 minutes, and washing buffer was PBS, pH 7.4 with 0.05% Tween 20. The array was pre-stained with the secondary antibodies as described below, to determine background staining. Rabbit KD4-2H4 (2.5 ug/ml) was incubated with the array for 16 hours at 4° C. in washing buffer with 10% blocking buffer with shaking at 140 rpm. Secondary antibody incubations were then performed with sheep anti-rabbit IgG (H+L) DyLight 680 (1:5000) for 45 minutes in washing buffer with 10% blocking buffer at room temperature. Control antibody was added to the secondary antibody incubations [mouse monoclonal anti-hemaggutinin (12CA5) DyLight800 (1:2000)]. Fluorescence intensity was read on a LI-COR Odessey Imaging System (scanning offset 0.65 mm, resolution 21 um, scanning intensities of 7/7 (red=700 nm/green=800 nm). Microarray image analysis was done with PepSlide® Analyzer. To identify common motifs MEME bioinformatics analysis was performed on the top hits with the highest spot intensities (available on the World Wide Web at meme.nbcr.net/meme/intro.html). MEME represents motifs as position-dependent letter-probability matrices which describe the probability of each possible letter at each position in the pattern. The MEME pre-settings were a maximum of one motif each sequence and of maximum three different motifs, as well as a minimum motif length of 5 amino acids. The e-value corresponds to the statistical significance of a consensus motif with the given log likelihood ratio (or higher) and with the same width and site count that one would find in a similarly sized set of random sequences. An e-value of 1 would be expected for the identification of a certain motif in a set of random peptides by chance.

Substitution analysis. Substitution analysis was performed on viral peptides recognized by antibodies KD4-2H4 and KD6-2B2 by creating a peptide array that includes stepwise substitution of all amino acid positions of the peptide with all 20 amino acids, to determine the amino acids that yielded optimal binding to the antibody (PEPperPRINT). Methods were identical to those described for the animal virus peptide array, with the exception that antibody KD4-2H4 was tested at 10 ug/ml and antibody KD6-2B2 was tested at 100 ug/ml.

ELISA for binding of peptides to KD monoclonal antibodies. Maxisorp Nunc Immuno 96-well plates were coated with 1 ug of synthetic peptides (Anaspec) per well and incubated with rabbit KD monoclonal antibodies at 10, 1, and 0.1p g/ml followed by horseradish peroxidase-labelled goat anti-rabbit antibody at 1:3000 (Fisher). Absorbance at 450 nm was determined on a Multiskan FC spectrophotometer after addition of Ultra 3,3′,5,5′-tetramethylbenzidine followed by 1.5M sulfuric acid solution. Absorbance of the KD peptide (KPAVIPDREALYQDIDEMEEC, SEQ ID NO:210) assays were recorded after subtraction of absorbance with the scrambled peptide (IYPLEDMAEPKVERIDAQEDC, SEQ ID NO:211). An OD reading >0.4 was arbitrarily determined as a positive; all negative antibodies had values of ≤0.04.

Blocking experiments to determine specificity of peptide binding. Monoclonal antibodies showing binding to animal virus peptides were incubated with a five-fold excess (by weight) of representative peptide or a scrambled peptide at 37° C. for 45 minutes, and each mixture was then applied to KD lung tissue for immunohistochemistry.

Testing of monoclonal antibodies for polyreactivity. ELISA was performed on monoclonal antibodies at 10, 1, and 0.1 ug/ml in ELISA plates coated with 1 ug/ml per well single stranded DNA (Sigma D8899), insulin (Sigma I2643), and bovine serum albumin (Fraction V, Fisher BP1600) (56). As a positive control, antibody KD11-1C1 was used. This VH4-34 antibody was encoded by an IgM plasmablast isolated from KD patient 11 (Table 2B). It was produced as an IgG antibody and was found to be polyreactive in ELISA against multiple diverse peptides, as is typical of many VH4-34 IgM antibodies (57).

Human protein array analyses and human protein immunohistochemistry. KD monoclonal antibodies human KD4-2H4 and rabbit KD6-2B2 were incubated at a concentration of 1 μg/ml at 4° C. overnight on a human proteome array (HuProt™v4.0, CDI Laboratories) which includes 16,793 genes, covering ˜80% of the canonical proteome as defined by the Human Protein Atlas (available on the World Wide Web at proteinatlas.org). Arrays were then probed with Alexa-647-anti-human IgG Fc gamma and Alexa-555-anti-rabbit secondary antibodies and imaged by CDI laboratories. Polyclonal rabbit antibody to integral membrane protein 2B (ITM2B) validated by the Human Protein Atlas (available on the World Wide Web at proteinatlas.org, HPA029292, Sigma) was used at the recommended 1:200 dilution for immunohistochemistry experiments including blocking experiments.

Western blot assay using glutathione sulfur transferase (GST)-concatemerized KD peptide fusion protein. We optimized the nucleotide sequence that codes for the KD peptide sequence for expression in E. coli and prepared a multimer with three copies of the peptide linked by short spacers [GST3X:AGKPAVIPDREALYQDIDEMEECLDEAGKPAVIPDREALYQDIDEMEECLDEAGKPA VIPDREALYQDIDEMEECLD (SEQ ID NO:191)]. This method of using a concatemerized fusion protein was shown to enhance diagnostic assays for Zika virus infection (58). The multimer sequence was cloned into the pGEX-KG plasmid (ATCC #77103) and fusion protein expression was induced from the recombinant vector and the original expression vector with 100 nM isopropyl β-d-1-thiogalactopyranoside (Goldbio, I248C) for 1.5 hrs at 30° C. (59). Bacteria were pelleted and resuspended in phosphate buffered saline and subjected to sonication (Branson digital sonifier SFX 550, Emerson), with cycles of 10 second sonication, 30 seconds of rest, for 15 cycles. The cell debris was pelleted by centrifugation at 15,000×g for 10 minutes. The cell-free supernatant was filtered using a 0.45p filter. The GST protein in the filtrate was purified using GSTrap FF (GE, 17513001) and eluted with 10 mM reduced glutathione (Sigma G4251), 50 mM Tris-HCl buffer pH 8.0. Purified proteins were quantified by BCA assay (Cat #23225, Thermofisher) and visualized by Coomassie staining. Western blot assays were performed following electrophoresis on 12% Tris-Glycine gels (Biorad) and transfer to PDVF membrane (Fisher IPVH00010). Blocking was with 5% nonfat dry milk in Tris buffered saline with 1% Tween 20 (TBST). Serum samples from KD patients and controls were diluted in TBST containing 5% nonfat dry milk at 1:5000 and incubated with membranes overnight at 4° C. Following incubation, membranes were washed five times in TBST, followed by incubation with horseradish peroxidase labelled goat anti-human IgG (Thermo, A18811) at a dilution of 1:5000 in phosphate buffered saline. After washing again five times in TBST, Supersignal West Femto Substrate (ThermoFisher, 34096) was applied, and blots were imaged using a ChemiDoc MP Imaging System (Biorad). Human immunoglobulin for intravenous use (IVIG) (Gammagard 10%, lot LE12T120AB) was assayed under the same conditions, but results were indeterminate because of reactivity with both GST and GST-3X. On each blot, lane 1 was GST alone (500 ng), lane 2 was GST-3× (500 ng), and lane 3 was human IgG (10-20 ng, Sigma 12511) used as a positive control for the binding of the secondary antibody. Blots were stripped and incubated with polyclonal rabbit anti-GST antibody (Fisher #717500, 1:1000) to ensure that both GST proteins were strongly detected after transfer. Sera found to be reactive with both GST and GST-3× were considered indeterminate and excluded from analyses (˜25% of all KD and control sera tested).

Statistical analysis. GraphPad Prism 8 was used to plot ELISA results. Comparison of serologic results between groups was performed using a two-tailed Fisher exact test using the function fishertest in R 3.6.1.

Human subjects. This study was approved by the Institutional Review Board of the Ann & Robert H. Lurie Children's Hospital of Chicago, and patients were enrolled following informed consent.

### REFERENCES

1. Yanagawa H, Nakamura Y, Yashiro M, Fujita Y, Nagai M, Kawasaki T, et al. A nationwide incidence survey of Kawasaki disease in 1985-1986 in Japan. J Infect Dis. 1988; 158(6):1296-301.
2. Yanagawa H, Nakamura Y, Kawasaki T, and Shigematsu I. Nationwide epidemic of Kawasaki disease in Japan during winter of 1985-86. Lancet. 1986; 2(8516):1138-9.
3. McCrindle B W, Rowley A H, Newburger J W, Burns J C, Bolger A F, Gewitz M, et al. Diagnosis, Treatment, and Long-Term Management of Kawasaki Disease: A Scientific Statement for Health Professionals From the American Heart Association. Circulation. 2017; 135(17):e927-e99.
4. Newburger J W, Takahashi M, Beiser A S, Burns J C, Bastian J, Chung K J, et al. A single intravenous infusion of gamma globulin as compared with four infusions in the treatment of acute Kawasaki syndrome. N Engl J Med. 1991; 324(23):1633-9.
5. Onouchi Y, Gunji T, Burns J C, Shimizu C, Newburger J W, Yashiro M, et al. ITPKC functional polymorphism associated with Kawasaki disease susceptibility and formation of coronary artery aneurysms. Nat Genet. 2008; 40(1):35-42.
6. Makino N, Nakamura Y, Yashiro M, Sano T, Ae R, Kosami K, et al. Epidemiological observations of Kawasaki disease in Japan, 2013-2014. Pediatr Int. 2018; 60(6):581-7.
7. Fan Q, Nelson C S, Bialas K M, Chiuppesi F, Amos J, Gurley T C, et al. Plasmablast Response to Primary Rhesus Cytomegalovirus (CMV) Infection in a Monkey Model of Congenital CMV Transmission. Clin Vaccine Immunol. 2017; 24(5).
8. Chai N, Swem L R, Park S, Nakamura G, Chiang N, Estevez A, et al. A broadly protective therapeutic antibody against influenza B virus with two mechanisms of action. Nat Commun. 2017; 8:14234.
9. Kauffman R C, Bhuiyan T R, Nakajima R, Mayo-Smith L M, Rashu R, Hoq M R, et al. Single-Cell Analysis of the Plasmablast Response to Vibrio cholerae Demonstrates Expansion of Cross-Reactive Memory B Cells. MBio. 2016; 7(6).
10. Garcia M, Iglesias A, Landoni V I, Bellomo C, Bruno A, Cordoba M T, et al. Massive plasmablast response elicited in the acute phase of hantavirus pulmonary syndrome. Immunology. 2017; 151(1):122-35.
11. Tanko R F, Soares A P, Muller T L, Garrett N J, Samsunder N, Abdool Karim Q, et al. Effect of Antiretroviral Therapy on the Memory and Activation Profiles of B Cells in HIV-Infected African Women. J Immunol. 2017; 198(3):1220-8.
12. Priyamvada L, Quicke K M, Hudson W H, Onlamoon N, Sewatanon J, Edupuganti S, et al. Human antibody responses after dengue virus infection are highly cross-reactive to Zika virus. Proc Natl Acad Sci USA. 2016; 113(28):7852-7.
13. Priyamvada L, Cho A, Onlamoon N, Zheng N Y, Huang M, Kovalenkov Y, et al. B Cell Responses during Secondary Dengue Virus Infection Are Dominated by Highly Cross-Reactive, Memory-Derived Plasmablasts. J Virol. 2016; 90(12):5574-85.
14. Carter M J, Mitchell R M, Meyer Sauteur P M, Kelly D F, and Truck J. The Antibody-Secreting Cell Response to Infection: Kinetics and Clinical Applications. Frontiers in immunology. 2017; 8:630.
15. Chen Y Q, Wohlbold T J, Zheng N Y, Huang M, Huang Y, Neu K E, et al. Influenza Infection in Humans Induces Broadly Cross-Reactive and Protective Neuraminidase-Reactive Antibodies. Cell. 2018; 173(2):417-29 e10.
16. Ellebedy A H, Jackson K J, Kissick H T, Nakaya H I, Davis C W, Roskin K M, et al. Defining antigen-specific plasmablast and memory B cell subsets in human blood after viral infection or vaccination. Nat-Immunol. 2016; 17(10):1226-34.
17. Regules J A, Cicatelli S B, Bennett J W, Paolino K M, Twomey P S, Moon J E, et al. Fractional Third and Fourth Dose of RTS,S/AS01 Malaria Candidate Vaccine: A Phase 2a Controlled Human Malaria Parasite Infection and Immunogenicity Study. J Infect Dis. 2016; 214(5):762-71.
18. Shaw J M, Miller-Novak L K, Mohanram V, McKinnon K, Demberg T, Vargas-Inchaustegui D A, et al. Influence of Plasma Cell Niche Factors on the Recruitment and Maintenance of IRF4hi Plasma Cells and Plasmablasts in Vaccinated, Simian Immunodeficiency Virus-Infected Rhesus Macaques with Low and High Viremia. J Virol. 2017; 91(4).
19. Stanfield B A, Pahar B, Chouljenko V N, Veazey R, and Kousoulas K G. Vaccination of rhesus macaques with the live-attenuated HSV-1 vaccine VC2 stimulates the proliferation of mucosal T cells and germinal center responses resulting in sustained production of highly neutralizing antibodies. Vaccine. 2017; 35(4):536-43.
20. Pitisuttithum P, Boonnak K, Chamnanchanunt S, Puthavathana P, Luvira V, Lerdsamran H, et al. Safety and immunogenicity of a live attenuated influenza H5 candidate vaccine strain A/17/turkey/Turkey/05/133 H5N2 and its priming effects for potential pre-pandemic use: a randomised, double-blind, placebo-controlled trial. Lancet Infect Dis. 2017.
21. Kim J H, Mishina M, Chung J R, Cole K S, Nowalk M P, Martin J M, et al. Cell-Mediated Immunity Against Antigenically Drifted Influenza A (H3N2) Viruses in Children During a Vaccine Mismatch Season. J Infect Dis. 2016; 214(7):1030-8.
22. Mei H E, Hahne S, Redlin A, Hoyer B F, Wu K, Baganz L, et al. Plasmablasts with a mucosal phenotype contribute to plasmacytosis in SLE. Arthritis Rheumatol. 2017.
23. Kinslow J D, Blum L K, Deane K D, Demoruelle M K, Okamoto Y, Parish M C, et al. Elevated IgA Plasmablast Levels in Subjects at Risk of Developing Rheumatoid Arthritis. Arthritis Rheumatol. 2016; 68(10):2372-83.
24. Wrammert J, Smith K, Miller J, Langley W A, Kokko K, Larsen C, et al. Rapid cloning of high-affinity human monoclonal antibodies against influenza virus. Nature. 2008; 453(7195):667-71.
25. Tan Y C, Blum L K, Kongpachith S, Ju C H, Cai X, Lindstrom T M, et al. High-throughput sequencing of natively paired antibody chains provides evidence for original antigenic sin shaping the antibody response to influenza vaccination. Clin Immunol. 2014; 151(1):55-65.
26. Lu D R, Tan Y C, Kongpachith S, Cai X, Stein E A, Lindstrom T M, et al. Identifying functional anti-Staphylococcus aureus antibodies by sequencing antibody repertoires of patient plasmablasts. Clin Immunol. 2014; 152(1-2):77-89.
27. Wrammert J, Onlamoon N, Akondy R S, Perng G C, Polsrila K, Chandele A, et al. Rapid and massive virus-specific plasmablast responses during acute dengue virus infection in humans. J Virol. 2012; 86(6):2911-8.
28. Rowley A H, Shulman S T, Spike B T, Mask C A, and Baker S C. Oligoclonal IgA response in the vascular wall in acute Kawasaki disease. J Immunol. 2001; 166(2):1334-43.
29. Rowley A H, Baker S C, Shulman S T, Garcia F L, Guzman-Cottrill J A, Chou P, et al. Detection of antigen in bronchial epithelium and macrophages in acute Kawasaki disease by use of synthetic antibody. J Infect Dis. 2004; 190(4):856-65.
30. Rowley A H, Shulman S T, Garcia F L, Guzman-Cottrill J A, Miura M, Lee H L, et al. Cloning the arterial IgA antibody response during acute Kawasaki disease. J Immunol. 2005; 175(12):8386-91.
31. Rowley A H, Baker S C, Shulman S T, Fox L M, Takahashi K, Garcia F L, et al. Cytoplasmic inclusion bodies are detected by synthetic antibody in ciliated bronchial epithelium during acute Kawasaki disease. J Infect Dis. 2005; 192(10):1757-66.
32. Fink K. Origin and Function of Circulating Plasmablasts during Acute Viral Infections. Frontiers in immunology. 2012; 3:78.
33. DeKosky B J, Lungu O I, Park D, Johnson E L, Charab W, Chrysostomou C, et al. Large-scale sequence and structural comparisons of human naive and antigen-experienced antibody repertoires. Proc Natl Acad Sci USA. 2016; 113(19):E2636-45.
34. Georgiou G, Ippolito G C, Beausang J, Busse C E, Wardemann H, and Quake S R. The promise and challenge of high-throughput sequencing of the antibody repertoire. Nat Biotechnol. 2014; 32(2):158-68.
35. Rowley A H, Baker S C, Shulman S T, Rand K H, Tretiakova M S, Perlman E J, et al. Ultrastructural, immunofluorescence, and RNA evidence support the hypothesis of a “new” virus associated with Kawasaki disease. J Infect Dis. 2011; 203(7):1021-30.
36. Rowley A H, Baker S C, Shulman S T, Garcia F L, Fox L M, Kos I M, et al. RNA-containing cytoplasmic inclusion bodies in ciliated bronchial epithelium months to years after acute Kawasaki disease. PLoS ONE. 2008; 3(2):e1582.
37. Forsstrom B, Axnas B B, Stengele K P, Buhler J, Albert T J, Richmond T A, et al. Proteome-wide epitope mapping of antibodies using ultra-dense peptide arrays. Molecular & cellular proteomics: MCP. 2014; 13(6):1585-97.
38. Rowley A H, Wylie K M, Kim K Y, Pink A J, Yang A, Reindel R, et al. The transcriptional profile of coronary arteritis in Kawasaki disease. BMC Genomics. 2015; 16(1):1076.
39. Matsuda S, and Senda T. BRI2 as an anti-Alzheimer gene. Med Mol Morphol. 2019; 52(1):1-7.
40. Rowley A H, and Shulman S T. The Epidemiology and Pathogenesis of Kawasaki Disease. Front Pediatr. 2018; 6:374.
41. Cameron S A, Carr M, Pahl E, DeMarais N, Shulman S T, and Rowley A H. Coronary artery aneurysms are more severe in infants than in older children with Kawasaki disease. Arch Dis Child. 2019; 104(5):451-5.
42. Makino N, Nakamura Y, Yashiro M, Kosami K, Matsubara Y, Ae R, et al. Nationwide epidemiologic survey of Kawasaki disease in Japan, 2015-2016. Pediatr Int. 2019; 61(4):397-403.
43. Maddox R A, Holman R C, Uehara R, Callinan L S, Guest J L, Schonberger L B, et al. Recurrent Kawasaki disease: USA and Japan. Pediatr Int. 2015; 57(6):1116-20.
44. Bell D M, Brink E W, Nitzkin J L, Hall C B, Wulff H, Berkowitz I D, et al. Kawasaki syndrome: description of two outbreaks in the United States. N Engl J Med. 1981; 304(26):1568-75.
45. Dean A G, Melish M E, Hicks R, and Palumbo N E. An epidemic of Kawasaki syndrome in Hawaii. J Pediatr. 1982; 100(4):552-7.
46. Salo E, Pelkonen P, and Pettay O. Outbreak of Kawasaki syndrome in Finland. Acta Paediatr Scand. 1986; 75(1):75-80.
47. Rowley A H, Eckerley C A, Jack H M, Shulman S T, and Baker S C. IgA plasma cells in vascular tissue of patients with Kawasaki syndrome. J Immunol. 1997; 159(12):5946-55.
48. Rowley A H, Shulman S T, Mask C A, Finn L S, Terai M, Baker S C, et al. IgA plasma cell infiltration of proximal respiratory tract, pancreas, kidney, and coronary artery in acute Kawasaki disease. J Infect Dis. 2000; 182(4):1183-91.
49. Hartlage A S, Cullen J M, and Kapoor A. The Strange, Expanding World of Animal Hepaciviruses. Annu Rev Virol. 2016; 3(1):53-75.
50. El-Attar L M, Mitchell J A, Brooks Brownlie H, Priestnall S L, and Brownlie J. Detection of non-primate hepaciviruses in UK dogs. Virology. 2015; 484:93-102.
51. Pfaender S, Cavalleri J M, Walter S, Doerrbecker J, Campana B, Brown R J, et al. Clinical course of infection and viral tissue tropism of hepatitis C virus-like nonprimate hepaciviruses in horses. Hepatology. 2015; 61(2):447-59.
52. Kawasaki T. [Acute febrile mucocutaneous syndrome with lymphoid involvement with specific desquamation of the fingers and toes in children]. Arerugi. 1967; 16(3):178-222.
53. Kobayashi T, Saji T, Otani T, Takeuchi K, Nakamura T, Arakawa H, et al. Efficacy of immunoglobulin plus prednisolone for prevention of coronary artery abnormalities in severe Kawasaki disease (RAISE study): a randomised, open-label, blinded-endpoints trial. Lancet. 2012; 379(9826):1613-20.
54. Ho I Y, Bunker T T, Erickson S A, Neu K E, Huang M, Cortese M, et al. Refined protocol for generating monoclonal antibodies from single human and murine B cells. J Immunol Methods. 2016; 438:67-70.
55. Smith K, Garman L, Wrammert J, Zheng N Y, Capra J D, Ahmed R, et al. Rapid generation of fully human monoclonal antibodies specific to a vaccinating antigen. Nat Protoc. 2009; 4(3):372-84.
56. Wardemann H, Yurasov S, Schaefer A, Young J W, Meffre E, and Nussenzweig M C. Predominant autoantibody production by early human B cell precursors. Science. 2003; 301(5638):1374-7.
57. Pugh-Bernard A E, Silverman G J, Cappione A J, Villano M E, Ryan D H, Insel R A, et al. Regulation of inherently autoreactive VH4-34 B cells in the maintenance of human B cell tolerance. J Clin Invest. 2001; 108(7):1061-70.
58. Mishra N, Caciula A, Price A, Thakkar R, Ng J, Chauhan L V, et al. Diagnosis of Zika Virus Infection by Peptide Array and Enzyme-Linked Immunosorbent Assay. MBio. 2018; 9(2).
59. Guan K L, and Dixon J E. Eukaryotic proteins expressed in Escherichia coli: an improved thrombin cleavage and purification procedure of fusion proteins with glutathione S-transferase. Anal Biochem. 1991; 192(2):262-7.

We claim:

1. An isolated intracytoplasmic inclusion body (ICI) antibody or antigen binding fragment thereof comprising:
a heavy chain variable domain comprising
a CDRH1 region selected from the group consisting of 272, 278, 284, 290, 295, 301, 306, 311, 317, 322, 328, 334, and 340;
a CDRH2 region selected from the group consisting of 273, 279, 285, 291, 296, 302, 307, 312, 318, 323, 329, 335, and 341; and
a CDRH3 region selected from the group consisting of 274, 280, 286, 299, 297, 303, 308, 313, 319, 324, 330, 336, and 342; and
a light chain variable domain comprising
a CDRL1 region selected from the group consisting of 269, 275, 281, 287, 293, 298, 304, 309, 314, 320, 325, 331, and 337;
a CDRL2 region selected from the group consisting of 270, 276, 282, 288, 299, 315, 321, 326, 332, and 338; and
a CDRL3 region selected from the group consisting of 271, 277, 283, 289, 294, 300, 305, 310, 316, 327, 333, and 339.

2. The isolated ICI antibody or antigen binding fragment of claim 1, comprising:
(a) a heavy chain variable domain comprising a CDRH1 region consisting of SEQ ID NO:272, a CDRH2 region consisting of SEQ ID NO:273, and a CDRH3 region consisting of SEQ ID NO:274 and a light chain variable domain comprising a CDRL1 region consisting of SEQ ID NO:269, a CDRL2 region consisting of SEQ ID NO:270, and a CDRL3 region consisting of SEQ ID NO:271, or
(b) a heavy chain variable domain comprising a CDRH1 region consisting of SEQ ID NO:278, a CDRH2 region consisting of SEQ ID NO:279, and a CDRH3 region consisting of SEQ ID NO:280 and a light chain variable domain comprising a CDRL1 region consisting of SEQ ID NO:275, a CDRL2 region consisting of SEQ ID NO:276, and a CDRL3 region consisting of SEQ ID NO:277, or
(c) a heavy chain variable domain comprising a CDRH1 region consisting of SEQ ID NO:284, a CDRH2 region consisting of SEQ ID NO:285, and a CDRH3 region consisting of SEQ ID NO:286 and a light chain variable domain comprising a CDRL1 region consisting of SEQ ID NO:281, a CDRL2 region consisting of SEQ ID NO:282, and a CDRL3 region consisting of SEQ ID NO:283, or
(d) a heavy chain variable domain comprising a CDRH1 region consisting of SEQ ID NO:290, a CDRH2 region consisting of SEQ ID NO:291, and a CDRH3 region consisting of SEQ ID NO:292 and a light chain variable domain comprising a CDRL1 region consisting of SEQ ID NO:287, a CDRL2 region consisting of SEQ ID NO:288, and a CDRL3 region consisting of SEQ ID NO:289, or
(e) a heavy chain variable domain comprising a CDRH1 region consisting of SEQ ID NO:295, a CDRH2 region consisting of SEQ ID NO:296, and a CDRH3 region consisting of SEQ ID NO:297 and a light chain variable domain comprising a CDRL1 region consisting of SEQ ID NO:293, a CDRL2 region consisting of SEQ ID NO:282, and a CDRL3 region consisting of SEQ ID NO:294, or
(f) a heavy chain variable domain comprising a CDRH1 region consisting of SEQ ID NO:301, a CDRH2 region consisting of SEQ ID NO:302, and a CDRH3 region consisting of SEQ ID NO:303 and a light chain variable domain comprising a CDRL1 region consisting of SEQ ID NO:298, a CDRL2 region consisting of SEQ ID NO:299, and a CDRL3 region consisting of SEQ ID NO:300, or
(g) a heavy chain variable domain comprising a CDRH1 region consisting of SEQ ID NO:306, a CDRH2 region consisting of SEQ ID NO:307, and a CDRH3 region consisting of SEQ ID NO:308 and a light chain variable domain comprising a CDRL1 region consisting of SEQ ID NO:304, a CDRL2 region consisting of SEQ ID NO:282, and a CDRL3 region consisting of SEQ ID NO:305, or
(h) a heavy chain variable domain comprising a CDRH1 region consisting of SEQ ID NO:311, a CDRH2 region consisting of SEQ ID NO:312, and a CDRH3 region consisting of SEQ ID NO:313 and a light chain variable domain comprising a CDRL1 region consisting of SEQ ID NO:309, a CDRL2 region consisting of SEQ ID NO:276, and a CDRL3 region consisting of SEQ ID NO:310, or
(i) a heavy chain variable domain comprising a CDRH1 region consisting of SEQ ID NO:317, a CDRH2 region consisting of SEQ ID NO:318, and a CDRH3 region consisting of SEQ ID NO:319 and a light chain variable domain comprising a CDRL1 region consisting of SEQ ID NO:314, a CDRL2 region consisting of SEQ ID NO:315, and a CDRL3 region consisting of SEQ ID NO:316, or
(j) a heavy chain variable domain comprising a CDRH1 region consisting of SEQ ID NO:322, a CDRH2 region consisting of SEQ ID NO:323, and a CDRH3 region consisting of SEQ ID NO:324 and a light chain variable domain comprising a CDRL1 region consisting of SEQ ID NO:320, a CDRL2 region consisting of SEQ ID NO:321, and a CDRL3 region consisting of SEQ ID NO:316, or
(k) a heavy chain variable domain comprising a CDRH1 region consisting of SEQ ID NO:328, a CDRH2 region consisting of SEQ ID NO:329, and a CDRH3 region consisting of SEQ ID NO:330 and a light chain variable domain comprising a CDRL1 region consisting of SEQ ID NO:325, a CDRL2 region consisting of SEQ ID NO:326, and a CDRL3 region consisting of SEQ ID NO:327, or
(l) a heavy chain variable domain comprising a CDRH1 region consisting of SEQ ID NO:334, a CDRH2 region consisting of SEQ ID NO:335, and a CDRH3 region consisting of SEQ ID NO:336 and a light chain variable domain comprising a CDRL1 region consisting of SEQ ID NO:331, a CDRL2 region consisting of SEQ ID NO:332, and a CDRL3 region consisting of SEQ ID NO:333, or
(m) a heavy chain variable domain comprising a CDRH1 region consisting of SEQ ID NO:340, a CDRH2 region consisting of SEQ ID NO:341, and a CDRH3 region consisting of SEQ ID NO:342 and a light chain variable domain comprising a CDRL1 region consisting of SEQ ID NO:337, a CDRL2 region consisting of SEQ ID NO:338, and a CDRL3 region consisting of SEQ ID NO:339.

3. The isolated ICI antibody or antigen binding fragment of claim 1, comprising:
(a) a light chain variable domain selected from the group consisting of SEQ ID NOs:165, 167, 169, 171, 173, 175, 177, 179, 181, 183, 185, 187, and 189; and
(b) a heavy chain variable domain selected from the group consisting of SEQ ID NOs:166, 168, 170, 172, 174, 176, 178, 180, 182, 184, 186, 188, and 190.

4. The isolated ICI antibody of any of claims 1-3, wherein the heavy chain variable domain comprises a CDRH1 region consisting of SEQ ID NO:272, a CDRH2 region consisting of SEQ ID NO:273, and a CDRH3 region consisting of SEQ ID NO:274 and the light chain variable domain comprises a CDRL1 region consisting of SEQ ID NO:269, a CDRL2 region consisting of SEQ ID NO:270, and a CDRL3 region consisting of SEQ ID NO:271.

5. The isolated ICI antibody of claim 4, wherein the light chain variable domain is SEQ ID NO:165 and the heavy chain variable domain is SEQ ID NO:166.

6. The isolated ICI antibody of any of claims 1-3, wherein the heavy chain variable domain comprises a CDRH1 region consisting of SEQ ID NO:278, a CDRH2 region consisting of SEQ ID NO:279, and a CDRH3 region consisting of SEQ ID NO:280 and the light chain variable domain comprises a CDRL1 region consisting of SEQ ID NO:275, a CDRL2 region consisting of SEQ ID NO:276, and a CDRL3 region consisting of SEQ ID NO:277.

7. The isolated ICI antibody of claim 6, wherein the light chain variable domain is SEQ ID NO:167 and the heavy chain variable domain is SEQ ID NO:168.

8. The isolated ICI antibody of any of claims 1-3, wherein the heavy chain variable domain comprises a CDRH1 region consisting of SEQ ID NO:284, a CDRH2 region consisting of SEQ ID NO:285, and a CDRH3 region consisting of SEQ ID NO:286 and a light chain variable domain comprises a CDRL1 region consisting of SEQ ID NO:281, a CDRL2 region consisting of SEQ ID NO:282, and a CDRL3 region consisting of SEQ ID NO:283.

9. The isolated ICI antibody of claim 8, wherein the light chain variable domain is SEQ ID NO:169 and the heavy chain variable domain is SEQ ID NO:170.

10. The isolated ICI antibody of any of claims 1-3, wherein the heavy chain variable domain comprises a CDRH1 region consisting of SEQ ID NO:290, a CDRH2 region consisting of SEQ ID NO:291, and a CDRH3 region consisting of SEQ ID NO:292 and the light chain variable domain comprises a CDRL1 region consisting of SEQ ID NO:287, a CDRL2 region consisting of SEQ ID NO:288, and a CDRL3 region consisting of SEQ ID NO:289.

11. The isolated ICI antibody of claim 10, wherein the light chain variable domain is SEQ ID NO:171 and the heavy chain variable domain is SEQ ID NO:172.

12. The isolated ICI antibody of any of claims 1-3, wherein the heavy chain variable domain comprises a CDRH1 region consisting of SEQ ID NO:295, a CDRH2 region consisting of SEQ ID NO:296, and a CDRH3 region consisting of SEQ ID NO:297 and the light chain variable domain comprises a CDRL1 region consisting of SEQ ID NO:293, a CDRL2 region consisting of SEQ ID NO:282, and a CDRL3 region consisting of SEQ ID NO:294.

13. The isolated ICI antibody of claim 12, wherein the light chain variable domain is SEQ ID NO:173 and the heavy chain variable domain is SEQ ID NO:174.

14. The isolated ICI antibody of any of claims 1-3, wherein the heavy chain variable domain comprises a CDRH1 region consisting of SEQ ID NO:301, a CDRH2 region consisting of SEQ ID NO:302, and a CDRH3 region consisting of SEQ ID NO:303 and the light chain variable domain comprises a CDRL1 region consisting of SEQ ID NO:298, a CDRL2 region consisting of SEQ ID NO:299, and a CDRL3 region consisting of SEQ ID NO:300.

15. The isolated ICI antibody of claim 14, wherein the light chain variable domain is SEQ ID NO:175 and the heavy chain variable domain is SEQ ID NO:176.

16. The isolated ICI antibody of any of claims 1-3, wherein the heavy chain variable domain comprises a CDRH1 region consisting of SEQ ID NO:306, a CDRH2 region consisting of SEQ ID NO:307, and a CDRH3 region consisting of SEQ ID NO:308 and the light chain variable domain comprises a CDRL1 region consisting of SEQ ID NO:304, a CDRL2 region consisting of SEQ ID NO:282, and a CDRL3 region consisting of SEQ ID NO:305.

17. The isolated ICI antibody of claim 16, wherein the light chain variable domain is SEQ ID NO:177 and the heavy chain variable domain is SEQ ID NO:178.

18. The isolated ICI antibody of any of claims 1-3, wherein the heavy chain variable domain comprises a CDRH1 region consisting of SEQ ID NO:311, a CDRH2 region consisting of SEQ ID NO:312, and a CDRH3 region consisting of SEQ ID NO:313 and the light chain variable domain comprises a CDRL1 region consisting of SEQ ID NO:309, a CDRL2 region consisting of SEQ ID NO:276, and a CDRL3 region consisting of SEQ ID NO:310.

19. The isolated ICI antibody of claim 18, wherein the light chain variable domain is SEQ ID NO:179 and the heavy chain variable domain is SEQ ID NO:180.

20. The isolated ICI antibody of any of claims 1-3, wherein the heavy chain variable domain comprises a CDRH1 region consisting of SEQ ID NO:317, a CDRH2 region consisting of SEQ ID NO:318, and a CDRH3 region consisting of SEQ ID NO:319 and the light chain variable domain comprises a CDRL1 region consisting of SEQ ID NO:314, a CDRL2 region consisting of SEQ ID NO:315, and a CDRL3 region consisting of SEQ ID NO:316.

21. The isolated ICI antibody of claim 20, wherein the light chain variable domain is SEQ ID NO:181 and the heavy chain variable domain is SEQ ID NO:182.

22. The isolated ICI antibody of any of claims 1-3, wherein the heavy chain variable domain comprises a CDRH1 region consisting of SEQ ID NO:322, a CDRH2 region consisting of SEQ ID NO:323, and a CDRH3 region consisting of SEQ ID NO:324 and the light chain variable domain comprises a CDRL1 region consisting of SEQ ID NO:320, a CDRL2 region consisting of SEQ ID NO:321, and a CDRL3 region consisting of SEQ ID NO:316.

23. The isolated ICI antibody of claim 22, wherein the light chain variable domain is SEQ ID NO:183 and the heavy chain variable domain is SEQ ID NO:184.

24. The isolated ICI antibody of any of claims 1-3, wherein the heavy chain variable domain comprises a CDRH1 region consisting of SEQ ID NO:328, a CDRH2 region consisting of SEQ ID NO:329, and a CDRH3 region consisting of SEQ ID NO:330 and the light chain variable domain comprises a CDRL1 region consisting of SEQ ID NO:325, a CDRL2 region consisting of SEQ ID NO:326, and a CDRL3 region consisting of SEQ ID NO:327.

25. The isolated ICI antibody of claim 24, wherein the light chain variable domain is SEQ ID NO:185 and the heavy chain variable domain is SEQ ID NO:186.

26. The isolated ICI antibody of any of claims 1-3, wherein the heavy chain variable domain comprises a CDRH1 region consisting of SEQ ID NO:334, a CDRH2 region consisting of SEQ ID NO:335, and a CDRH3 region consisting of SEQ ID NO:336 and the light chain variable domain comprises a CDRL1 region consisting of SEQ ID NO:331, a CDRL2 region consisting of SEQ ID NO:332, and a CDRL3 region consisting of SEQ ID NO:333.

27. The isolated ICI antibody of claim 26, wherein the light chain variable domain is SEQ ID NO:187 and the heavy chain variable domain is SEQ ID NO:188.

28. The isolated ICI antibody of any of claims 1-3, wherein the heavy chain variable domain comprises a CDRH1 region consisting of SEQ ID NO:340, a CDRH2 region consisting of SEQ ID NO:341, and a CDRH3 region consisting of SEQ ID NO:342 and the light chain variable domain comprises a CDRL1 region consisting of SEQ ID NO:337, a CDRL2 region consisting of SEQ ID NO:338, and a CDRL3 region consisting of SEQ ID NO:339.

29. The isolated ICI antibody of claim 28, wherein the light chain variable domain is SEQ ID NO:189 and the heavy chain variable domain is SEQ ID NO:190.

30. The isolated ICI antibody of any of claims 1-29, wherein the antibody is a chimeric antibody and the heavy chain constant domain is from rabbit, mouse, rat, or nonhuman primate.

31. The isolated ICI antibody of any of claims 1-30, wherein the light chain constant domain is a kappa light chain constant domain or a lambda light chain constant domain.

32. The isolated ICI antibody of any of claims 1-31, wherein the antibody is biotinylated.

33. A method of diagnosing Kawasaki Disease in a subject, comprising the steps of:
i) obtaining a sample from a subject suspected of having Kawasaki Disease;
ii) contacting the sample with the antibody of any of claims 1-31; and
ii) detecting the binding of the antibody in the sample, whereby binding of the antibody indicates the presence of Kawasaki Disease.

34. The method of claim 33, wherein the sample is a blood sample.

35. The method of claim 33, wherein detecting the binding of the antibody in the sample is carried out using ELISA, Western blot, immunostaining, immunoprecipitation, flow cytometry, sensor chips, or magnetic beads.

36. A method of detecting intracytoplasmic inclusion bodies in a subject, comprising the steps of:
i) obtaining a sample from a subject suspected of having Kawasaki Disease;
ii) contacting the sample with the antibody of any of claims 1-31; and
ii) detecting the binding of the antibody in the sample, whereby binding of the antibody indicates the presence of intracytoplasmic inclusion bodies.

37. The method of claim 36, where in the sample is a blood sample.

38. A method of detecting hepacivirus C in a subject, comprising the steps of:
i) obtaining a sample form a subject suspected of having a hepacivirus C infection;
ii) contacting the sample with the antibody of any of claims 1-31; and
iii) detecting the binding of the antibody in the sample, wherein binding of the antibody indicates the presence of hepacivirus C infection.
